(19) United States
(12) Patent Application Publication
(10) Pub. No.: US 2023/0233474 A1
(43) Pub. Date: Jul. 27, 2023

(54) USE OF MRNAS ENCODING OX40L, IL-23 AND IL-36GAMMA FOR TREATING CANCER

(71) Applicant: ModernaTX, Inc., Cambridge, MA (US)

(72) Inventors: Robert MEEHAN, Cambridge, MA (US); Tal ZAKS, Cambridge, MA (US); Pamela COHEN, Cambridge, MA (US)

(21) Appl. No.: 17/927,940

(22) PCT Filed: May 28, 2021
(86) PCT No.: PCT/US2021/034858

§ 371 Date: Nov. 28, 2022

Related U.S. Application Data
(60) Provisional application No. 63/031,307, filed on May 28, 2020.

(51) Int. Cl. A61K 9/51; A61K 47/54; A61K 38/20; A61P 35/00

(52) U.S. Cl. CPC A61K 9/5123; A61K 47/549; A61K 38/20; A61P 35/00

(57) ABSTRACT

The disclosure features methods for treating solid tumor malignancies and lymphomas by administering LNP encapsulated mRNAs encoding human OX40L, IL-23 and IL-36γ polypeptides alone or in combination with checkpoint blockade. The disclosure also features compositions for use in the methods.

### CROSS-REFERENCE TO RELATED APPLICATIONS

This application claims the benefit of U.S. Provisional Patent Application Ser. No. 63/031,307 filed May 28, 2020. The entire contents of which is incorporated herein by reference.

### BACKGROUND

Cancer is a disease characterized by uncontrolled cell division and growth within the body. In the United States, roughly a third of all women and half of all men will experience cancer in their lifetime. Cancer cells utilize a number of mechanisms to evade the immune system, which results in persistence of tumor cells. Much research has focused on methods of stimulating the immune system to allow it to recognize and attack tumor cells. One area of intense research is the use of immune checkpoint blockade (CTLA-4, PD-1, and PD-L1) to turn on immune responses to tumor cells.

The tumor necrosis factor receptor superfamily also contains molecules which may be useful as immunomodulators. For example, several anti-OX40 antibodies have been tested in the clinic for their ability to treat cancer. However, it is often difficult to reconcile in vitro and in vivo data in this area of research. Further data from human patients is required to determine what immunomodulatory therapies or combinations thereof work best for what types of cancers; and what therapeutic modalities—protein therapy, gene therapy, mRNA therapy, or combinations thereof, are most effective.

### SUMMARY OF DISCLOSURE

The present disclosure is based, at least in part, on the discovery that messenger RNAs (mRNAs) encoding human OX40 ligand (OX40L), human IL-23 and human IL-36γ administered by intratumoral injection (ITu) to solid tumor malignancies or lymphoma resulted in a significant reduction in tumor size or complete resolution of a tumor at a treated or uninjected tumor. Significantly, a local regional abscopal effect was observed for proximal, uninjected tumors within the vicinity of the injection site. Without being bound by theory, it is believed that the induction of OX40L, IL-23 and IL-36γ expression by tumor cells and/or cells presenting tumor antigens following administration of mRNAs encoding human OX40L, IL-23 and IL-36γ induces a specific cell-mediated immune response with systemic anti-tumor effects, resulting in a reduction in tumor size of both treated and untreated tumors.

Systemic administration of checkpoint-inhibitor (CPI) antibodies such as T-cell co-inhibitor pathways of PD-1/PD-L1 and CTLA-4 has resulted in survival improvements in metastatic diseases with unmet medical need. Although these approaches have yielded responses in a variety of indications, there are a significant number of cancer patients whose tumors are resistant to CPIs. Combination therapies with systematically administered CPIs, although demonstrated improvements in patient responses, also displayed increased on-target anti-normal tissue toxicities. Thus, there still exists an unmet need for safe and effective cancer therapies.

Without being bound by theory, local administration of ITu immune-mediated therapies for cancer, such as mRNAs encoding human OX40L, IL-23 and IL-36γ, results in systemic immune recognition of tumoral antigens, which as monotherapy cause regression of multiple metastatic lesions. As such, the mRNA therapeutic agents can improve outcomes from systemically delivered antibodies, such as CPIs, and have an improved tolerability profile compared to systemic therapy alone.

As demonstrated herein, ITu administration of mRNAs encoding human OX40L, IL-23 and IL-36γ alone or in combination with durvalumab, a CPI, resulted in reductions of tumor size in several patients having different types of cancer. Also demonstrated is reduction of tumor size when patients are administered the LNP-encapsulated mRNAs in a first dosing cycle and at least one subsequent dosing cycle, where the first dosing cycle is different from the subsequent dosing cycle(s) (e.g., a first dosing cycle of once weekly administration of the LNP-encapsulated mRNAs or once every 2 weeks, followed by at least one subsequent dosing cycle of administration of the LNP-encapsulated mRNA once every 4 weeks). Further, increases in IL-23 and IL-36γ protein expression are observed in both plasma and tumor biopsies after treatment, along with increased expression of downstream cytokines IL-22 and IL-6. It has also been shown that post-treatment cytokine levels (including TNFα and IFNγ) are well below what has been suggested as clinically toxic levels in cytokine release syndrome. It has also been demonstrated that mRNAs encoding OX40L, IL-23 and IL-36γ alone or in combination with durvalumab results in increased PD-L1 expression, which persists for up to two weeks in some patients. In addition, it has been shown that proliferation of T cells, particularly CD8+ T cells, is increased in the tumor microenvironment up to a month after treatment.

Resistance to CPIs has been reported for several cancers, including melanoma and non-small cell lung carcinoma. At least because mRNAs encoding human OX40L, IL-23 and IL-36γ are shown herein to increase PD-L1 expression and induce an immune response, without being bound by theory it is believed LNP-encapsulated mRNAs encoding human OX40L, IL-23 and IL-36γ provide anti-tumor efficacy against cancers that are primary refractory or secondary acquired to prior CPI therapy, alone or in combination with CPI therapy.

Accordingly, in some aspects the disclosure provides a method for treating solid tumor malignancies or lymphomas in a human patient by administering an effective amount of a pharmaceutical composition comprising: a lipid nanoparticle (LNP) comprising mRNAs encoding human OX40L, IL-23 and IL-36γ polypeptides; and a pharmaceutically acceptable carrier, thereby treating solid tumors or lymphomas in the patient. Without being bound by theory, delivery of mRNAs encoding human OX40L, IL-23 and IL-36γ polypeptides, which have distinct functions yet work synergistically, mediate anti-cancer responses either as a monotherapy or in combination with CPI antibodies (anti-PD-L1 and anti-CTLA-4).

In other aspects the disclosure provides a method for treating solid tumor malignancies or lymphomas in a human patient by inducing or enhancing an anti-tumor immune response, comprising administering to the patient by intratumoral injection an effective amount of a pharmaceutical composition comprising: a lipid nanoparticle (LNP) comprising mRNAs encoding human OX40L, IL-23 and IL-36γ polypeptides; and a pharmaceutically acceptable carrier, thereby treating solid tumors or lymphomas in the patient by inducing or enhancing an anti-tumor immune response. Without being bound by theory, ITu delivery of LNP encapsulated mRNAs encoding human OX40L, IL-23 and IL-36γ polypeptides, which have distinct functions yet work synergistically, mediate anti-cancer responses either as a monotherapy or in combination with CPI antibodies (anti-PD-L1 and anti-CTLA-4).

The present disclosure provides methods for treating advanced or metastatic solid tumor malignancy or lymphoma in a human patient by inducing or enhancing an anti-tumor immune response, comprising administering to the patient by intratumoral injection an effective amount of a lipid nanoparticle (LNP) encapsulated messenger RNA (mRNA) therapeutic agent comprising three mRNA drug substances:

(i) a first mRNA comprising an open reading frame (ORF) encoding a human OX40L polypeptide;

(ii) a second mRNA comprising an ORF encoding a human IL-23 polypeptide; and

(iii) a third mRNA comprising an ORF encoding a human IL-36γ polypeptide,

wherein the patient is administered a dose of 0.10-10.0 mg of the mRNA therapeutic agent in a dosing regimen from 7 to 28 days, thereby treating the advanced or metastatic solid tumor malignancy or lymphoma in the patient by inducing or enhancing an anti-tumor immune response.

In other aspects, present disclosure provides methods for treating advanced or metastatic solid tumor malignancy or lymphoma in a human patient, comprising administering to the patient, optionally by intratumoral injection, an effective amount of a lipid nanoparticle (LNP) encapsulated messenger RNA (mRNA) therapeutic agent comprising three mRNA drug substances:

(i) a first mRNA comprising an open reading frame (ORF) encoding a human OX40L polypeptide;

(ii) a second mRNA comprising an ORF encoding a human IL-23 polypeptide; and

(iii) a third mRNA comprising an ORF encoding a human IL-36γ polypeptide,

wherein the patient is administered a dose of 0.10-10.0 mg of the mRNA therapeutic agent in a dosing regimen from 7 to 28 days, thereby treating the advanced or metastatic solid tumor malignancy or lymphoma in the patient.

In some aspects, the present disclosure provides methods for treating advanced or metastatic solid tumor malignancy or lymphoma in a human patient, comprising administering to the patient an effective amount of a lipid nanoparticle (LNP) encapsulated messenger RNA (mRNA) therapeutic agent comprising three mRNA drug substances:

(i) a first mRNA comprising an open reading frame (ORF) encoding a human OX40L polypeptide;

(ii) a second mRNA comprising an ORF encoding a human IL-23 polypeptide; and

(iii) a third mRNA comprising an ORF encoding a human IL-36γ polypeptide,

wherein the patient is administered a dose of 0.10-10.0 mg of the mRNA therapeutic agent in a dosing regimen comprising a first dosing cycle and at least one subsequent dosing cycle, wherein the first and subsequent dosing cycles comprise administration of the mRNA therapeutic at a dosing interval selected from once a week, once every 2 weeks, once every 3 weeks, and once every 4 weeks for a duration of time, wherein the dosing intervals for the first and subsequent dosing cycles are different, thereby treating the advanced or metastatic solid tumor malignancy or lymphoma in the patient.

In some aspects, the present disclosure provides methods for treating advanced or metastatic solid tumor malignancy or lymphoma in a human patient, comprising administering to the patient by intratumoral injection an effective amount of a lipid nanoparticle (LNP) encapsulated messenger RNA (mRNA) therapeutic agent comprising three mRNA drug substances:

(i) a first mRNA comprising an open reading frame (ORF) encoding a human OX40L polypeptide;

(ii) a second mRNA comprising an ORF encoding a human IL-23 polypeptide; and

(iii) a third mRNA comprising an ORF encoding a human IL-36γ polypeptide,

wherein the patient is administered a dose of 0.10-10.0 mg of the mRNA therapeutic agent in a dosing regimen comprising a first dosing cycle and at least one subsequent dosing cycle, wherein the first and subsequent dosing cycles comprise administration of the mRNA therapeutic at a dosing interval selected from once a week, once every 2 weeks, once every 3 weeks, and once every 4 weeks for a duration of time, wherein the dosing intervals for the first and subsequent dosing cycles are different, thereby treating the advanced or metastatic solid tumor malignancy or lymphoma in the patient.

In any of the foregoing or related aspects, the first dosing cycle comprises administration of the dose of the mRNA therapeutic at a dosing interval of once a week for a duration of time. In some aspects, the first dosing cycle comprises administration of the dose of the mRNA therapeutic at a dosing interval of once every 2 weeks for a duration of time. In some aspects, the first dosing cycle comprises administration of the dose of the mRNA therapeutic at a dosing interval of once a week for a duration of time, and at least one subsequent dosing cycle comprising administration of the dose of the mRNA therapeutic at a dosing interval of once every 2 weeks, once every 3 weeks, or once every 4 weeks for a duration of time. In some aspects, the at least one subsequent dosing cycle comprises administration of the dose of the mRNA therapeutic at a dosing interval of once every 4 weeks for a duration of time. In some aspects, the first dosing cycle comprises administration of the dose of the mRNA therapeutic at a dosing interval of once every 2 weeks for a duration of time, and at least one subsequent dosing cycle comprising administration of the dose of the mRNA therapeutic at a dosing interval of once every 4 weeks for a duration of time. In some aspects, the first dosing cycle comprises administration of the dose of the mRNA therapeutic at a dosing interval of once every 2 weeks for a duration of time, and up to six subsequent dosing cycles each comprising administration of the dose of the mRNA therapeutic at a dosing interval of once every 4 weeks for a duration of time.

In any of the foregoing or related aspects, the duration of time for the first and subsequent dosing cycles is 28-42 days. In some aspects, the duration of time for the first and subsequent dosing cycles is 28 days. In some aspects, the duration of time for the first and subsequent dosing cycles is 35 days. In some aspects, the duration of time for the first and subsequent dosing cycles is 42 days.

In some aspects, the first dosing cycle comprises administration of the dose of the mRNA therapeutic at a dosing interval of once a week for 28-42 days. In some aspects, the first dosing cycle comprises administration of the dose of the mRNA therapeutic at a dosing interval of once every 2 weeks for 28-42 days. In some aspects, the first dosing cycle comprises administration of the dose of the mRNA therapeutic at a dosing interval of once weekly for 28-42 days, and at least one subsequent dosing cycle comprising administration of the dose of the mRNA therapeutic at a dosing interval of once every 2 weeks, once every 3 weeks, or once every 4 weeks for 28-42 days. In some aspects, the at least one subsequent dosing cycle comprises administration of the dose of the mRNA therapeutic at a dosing interval of once every 4 weeks for 28-42 days. In some aspects, the first dosing cycle comprises administration of the dose of the mRNA therapeutic at a dosing interval of once every 2 weeks for 28-42 days, and at least one subsequent dosing cycle comprising administration of the dose of the mRNA therapeutic at a dosing interval of once every 4 weeks for 28-42 days. In some aspects, the first dosing cycle comprises administration of the dose of the mRNA therapeutic at a dosing interval of once every 2 weeks for 28-42 days, and up to six subsequent dosing cycles each comprising administration of the dose of the mRNA therapeutic at a dosing interval of once every 4 weeks for 28-42 days. In some aspects, the first dosing cycle comprises administration of the dose of the mRNA therapeutic at a dosing interval of once every 2 weeks for 28 days, and at least one subsequent dosing cycle comprising administration of the dose of the mRNA therapeutic at a dosing interval of once every 4 weeks for 28 days. In some aspects, the first dosing cycle comprises administration of the dose of the mRNA therapeutic at a dosing interval of once every 2 weeks for 28 days, and up to six subsequent dosing cycles each comprising administration of the dose of the mRNA therapeutic at a dosing interval of once every 4 weeks for 28 days.

In some aspects of the methods of the present disclosure, the patient is administered a dose of the mRNA therapeutic agent selected from: 0.25-8.0 mg; 0.25-4.0 mg; 0.25-2.0 mg; 0.25-1.0 mg; 0.25-5 mg; 0.5-8.0 mg; 0.5-4.0 mg; 0.5-2.0 mg; 0.5-1.0 mg; 1.0-8.0 mg; 1.0-4.0 mg; 1.0-2.0 mg; 2.0-8.0 mg; 2.0-4.0 mg; and 4.0-8.0 mg. In some aspects, the patient is administered a dose of 0.10 mg of the mRNA therapeutic agent. In some aspects, the patient is administered a dose of 0.25 mg of the mRNA therapeutic agent. In some aspects, the patient is administered a dose of 0.50 mg of the mRNA therapeutic agent. In some aspects, the patient is administered a dose of 1.0 mg of the mRNA therapeutic agent. In some aspects, the patient is administered a dose of 2.0 mg of the mRNA therapeutic agent. In some aspects, the patient is administered a dose of 4.0 mg of the mRNA therapeutic agent. In some aspects, the patient is administered a dose of 8.0 mg of the mRNA therapeutic agent. In some aspects, the patient is administered a dose of 10.0 mg of the mRNA therapeutic agent.

In any of the foregoing or related aspects of the methods of the present disclosure, the mRNA therapeutic agent is administered to the patient in a dosing regimen selected from 7 to 21 days, 7 to 14 days, 28 days, 21 days, 14 days, and 7 days. In some aspects, the patient is administered a dose of the mRNA therapeutic agent every 2 weeks. In some aspects, the patient is administered a dose of the mRNA therapeutic agent every 3 weeks. In some aspects, the patient is administered a dose of the mRNA therapeutic agent every 4 weeks.

In some aspects, the disclosure provides a method for treating advanced or metastatic solid tumor malignancy or lymphoma in a human patient by inducing or enhancing an anti-tumor immune response, comprising administering to the patient by intratumoral injection an effective amount of an LNP encapsulated mRNA therapeutic agent comprising three mRNA drug substances:

(i) a first mRNA comprising an ORF encoding a human OX40L polypeptide;

(ii) a second mRNA comprising an ORF encoding a human IL-23 polypeptide; and

(iii) a third mRNA comprising an ORF encoding a human IL-36γ polypeptide,

wherein the patient is administered a dose of the mRNA therapeutic agent selected from 0.25 mg, 0.5 mg, 1 mg, 2 mg, 4 mg, and 8 mg, thereby treating the advanced or metastatic solid tumor malignancy or lymphoma in the patient by inducing or enhancing an anti-tumor immune response.

In some aspects, the disclosure provides a method for treating advanced or metastatic solid tumor malignancy or lymphoma in a human patient, comprising administering to the patient by, optionally by intratumoral injection, an effective amount of an LNP encapsulated mRNA therapeutic agent comprising three mRNA drug substances:

(i) a first mRNA comprising an ORF encoding a human OX40L polypeptide;

(ii) a second mRNA comprising an ORF encoding a human IL-23 polypeptide; and

(iii) a third mRNA comprising an ORF encoding a human IL-36γ polypeptide,

wherein the patient is administered a dose of the mRNA therapeutic agent selected from 0.25 mg, 0.5 mg, 1 mg, 2 mg, 4 mg, and 8 mg, thereby treating the advanced or metastatic solid tumor malignancy or lymphoma in the patient.

In some aspects, the patient is administered a dose of 0.25 mg of the mRNA therapeutic agent. In some aspects, the patient is administered a dose of 0.50 mg of the mRNA therapeutic agent. In some aspects, the patient is administered a dose of 1.0 mg of the mRNA therapeutic agent. In some aspects, the patient is administered a dose of 2.0 mg of the mRNA therapeutic agent. In some aspects, the patient is administered a dose of 4.0 mg of the mRNA therapeutic agent. In some aspects, the patient is administered a dose of 8.0 mg of the mRNA therapeutic agent.

In any of the foregoing or related aspects of the methods of the present disclosure, the patient is administered a dose of the mRNA therapeutic agent every 2 weeks. In some aspects, the patient is administered a dose of the mRNA therapeutic agent every 3 weeks. In some aspects, the patient is administered a dose of the mRNA therapeutic agent every 4 weeks.

In any of the foregoing or related aspects of the methods of the present disclosure, the patient is administered a dose of the mRNA therapeutic agent for at least two dosing cycles. In some embodiments, a dosing cycle comprises more than one dose. In some aspects, the patient is administered the mRNA therapeutic in a first dosing cycle comprising a dose every 2 weeks, and at least a second dosing cycle comprising a dose every 4 weeks. In some aspects, the patient is administered the mRNA therapeutic in a first dosing cycle comprising a dose every 2 weeks, and at least one subsequent dosing cycle comprising a dose every 4 weeks. In some aspects, the patient is administered the mRNA therapeutic in a first dosing cycle comprising a dose every 2 weeks, and at least two subsequent dosing cycles comprising a dose every 4 weeks.

In any of the foregoing or related aspects of the methods of the present disclosure, the patient is administered a second composition comprising an effective amount of a PD-1 antagonist, a PD-L1 antagonist or a CTLA-4 antagonist. In some aspects, the PD-1 antagonist is an antibody or antigen binding portion thereof that specifically binds to PD-1. In some aspects, the PD-1 antagonist is selected from the group consisting of nivolumab, pembrolizumab, and pidilizumab. In some aspects, the PD-L1 antagonist is an antibody or antigen binding portion thereof that specifically binds to PD-L1. In some aspects, the PD-L1 antagonist is selected from the group consisting of durvalumab, avelumab, and atezolizumab. In some aspects, the PD-L1 antagonist is durvalumab. In some aspects, the CTLA-4 antagonist is an antibody or antigen binding portion thereof that specifically binds to CTLA-4. In some aspects, the CTLA-4 antagonist is selected from the group consisting of ipilimumab and tremelimumab. In some aspects, the CTLA-4 antagonist is tremelimumab.

In any of the foregoing or related aspects of the methods of the present disclosure, the patient is administered a dose of an effective amount of a PD-1 antagonist, a PD-L1 antagonist or a CTLA-4 antagonist every 4 weeks. In some aspects, the patient is administered a dose of the mRNA therapeutic agent prior to administration of the PD-1 antagonist, PD-L1 antagonist or CTLA4 antagonist. In some aspects, the patient is administered a dose of the PD-1 antagonist, PD-L1 antagonist or CTLA4 antagonist prior to administration of the mRNA therapeutic agent. In some aspects, the patient is administered a dose of the mRNA therapeutic agent simultaneously with administration of a dose of the PD-1 antagonist, PD-L1 antagonist or CTLA4 antagonist.

In any of the foregoing or related aspects of the methods of the present disclosure, the advanced or metastatic solid tumor malignancy in the patient is selected from triple negative breast cancer, head and neck squamous cell carcinoma, bladder cancer (e.g., urothelial cancer), squamous-cell bladder cancer, bladder adenocarcinoma and melanoma and the lymphoma is Non-Hodgkin lymphoma. In some aspects, the advanced or metastatic solid tumor malignancy in the patient is selected from triple negative breast cancer, head and neck squamous cell carcinoma, bladder cancer, non-small cell lung carcinoma and melanoma, and the lymphoma is Non-Hodgkin lymphoma. In some aspects, the advanced or metastatic solid tumor malignancy in the patient is selected from triple negative breast cancer, head and neck squamous cell carcinoma, bladder cancer (e.g., urothelial cancer), squamous-cell bladder cancer, bladder adenocarcinoma, non-small cell lung carcinoma and melanoma and the lymphoma is Non-Hodgkin lymphoma. In some aspects, the advanced or metastatic solid tumor malignancy is triple negative breast cancer. In some aspects, the advanced or metastatic solid tumor malignancy is head and neck squamous cell carcinoma. In some aspects, the advanced or metastatic solid tumor malignancy is bladder cancer. In some aspects, the advanced or metastatic solid tumor malignancy is urothelial cancer. In some aspects, the advanced or metastatic solid tumor malignancy is squamous-cell bladder cancer. In some aspects, the advanced or metastatic solid tumor malignancy is bladder adenocarcinoma. In some aspects, the advanced or metastatic solid tumor malignancy is non-small cell lung carcinoma. In some aspects, the advanced or metastatic solid tumor malignancy is melanoma. In some aspects, the lymphoma is Non-Hodgkin lymphoma.

In any of the foregoing or related aspects, the advanced or metastatic solid tumor malignancy is refractory to immune checkpoint inhibitor therapy. In other aspects, the patient has not received anti-cancer treatment prior to administering the mRNA therapeutic.

In some aspects, the disclosure provides a method for treating advanced or metastatic solid tumor malignancy in a human patient, comprising administering to the patient an effective amount of a lipid nanoparticle (LNP) encapsulated messenger RNA (mRNA) therapeutic agent comprising three mRNA drug substances:

(i) a first mRNA comprising an open reading frame (ORF) encoding a human OX40L polypeptide;

(ii) a second mRNA comprising an ORF encoding a human IL-23 polypeptide; and

(iii) a third mRNA comprising an ORF encoding a human IL-36γ polypeptide,

wherein the patient is administered a dose of 0.10-10.0 mg of the mRNA therapeutic agent, wherein the solid tumor malignancy is selected from the group consisting of: a bladder cancer, an immune checkpoint inhibitor (CPI)-refractory melanoma, a neoadjuvant melanoma, and a non-small cell lung carcinoma, thereby treating the advanced or metastatic solid tumor malignancy in the patient.

In some aspects, the disclosure provides a method for treating advanced or metastatic solid tumor malignancy in a human patient, comprising administering to the patient by intratumoral injection an effective amount of a lipid nanoparticle (LNP) encapsulated messenger RNA (mRNA) therapeutic agent comprising three mRNA drug substances:

(i) a first mRNA comprising an open reading frame (ORF) encoding a human OX40L polypeptide;

(ii) a second mRNA comprising an ORF encoding a human IL-23 polypeptide; and

(iii) a third mRNA comprising an ORF encoding a human IL-36γ polypeptide,

wherein the patient is administered a dose of 0.10-10.0 mg of the mRNA therapeutic agent, wherein the solid tumor malignancy is selected from the group consisting of: a bladder cancer, an immune checkpoint inhibitor (CPI)-refractory melanoma, a neoadjuvant melanoma, and a non-small cell lung carcinoma, thereby treating the advanced or metastatic solid tumor malignancy in the patient.

In any of the foregoing or related aspects, the bladder cancer is (i) a urothelial cancer, optionally wherein the patient has received or is receiving platinum-based chemotherapy or the patient is ineligible for platinum-based chemotherapy; or (ii) a squamous-cell bladder cancer, optionally wherein the squamous-cell bladder cancer is PD-L1 negative or expresses low levels of PD-L1.

In any of the foregoing or related aspects, the bladder cancer or non-small cell lung carcinoma is refractory to immune checkpoint inhibitor therapy.

In some aspects, the disclosure provides a method for treating triple negative breast cancer (TNBC) in a human patient, comprising administering to the patient:

(a) an effective amount of an LNP encapsulated mRNA therapeutic agent comprising three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide; and

(b) an effective amount of PD-L1 antagonist selected from the group consisting of durvalumab, avelumab, and atezolizumab,

thereby treating the TNBC in the patient.

In some aspects, the disclosure provides a method for treating head and neck squamous cell carcinoma (HNSCC) in a human patient, comprising administering to the patient:

(a) an effective amount of an LNP encapsulated mRNA therapeutic agent comprising three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide; and

(b) an effective amount of PD-L1 antagonist selected from the group consisting of durvalumab, avelumab, and atezolizumab,

thereby treating the HNSCC in the patient.

In some aspects, the disclosure provides a method for treating Non-Hodgkin lymphoma (NHL) in a human patient, comprising administering to the patient:

(a) an effective amount of an LNP encapsulated mRNA therapeutic agent comprising three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide; and

(b) an effective amount of PD-L1 antagonist selected from the group consisting of durvalumab, avelumab, and atezolizumab,

thereby treating the NHL in the patient.

In some aspects, the disclosure provides a method for treating bladder cancer in a human patient, comprising administering to the patient:

(a) an effective amount of an LNP encapsulated mRNA therapeutic agent comprising three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide; and

(b) an effective amount of PD-L1 antagonist selected from the group consisting of durvalumab, avelumab, and atezolizumab,

thereby treating the bladder cancer in the patient.

In some aspects, the disclosure provides a method for treating urothelial cancer in a human patient, comprising administering to the patient:

(a) an effective amount of an LNP encapsulated mRNA therapeutic agent comprising three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide; and

(b) an effective amount of PD-L1 antagonist selected from the group consisting of durvalumab, avelumab, and atezolizumab,

thereby treating the urothelial cancer in the patient. In some aspects, the patient having urothelial cancer is ineligible for platinum-based chemotherapy. In some aspects, the patient having urothelial cancer is resistant for platinum-based chemotherapy. In some aspects, the patient having urothelial cancer has received or is receiving platinum-based chemotherapy.

In some aspects, the disclosure provides a method for treating squamous-cell bladder cancer in a human patient, comprising administering to the patient:

(a) an effective amount of an LNP encapsulated mRNA therapeutic agent comprising three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide; and

(b) an effective amount of PD-L1 antagonist selected from the group consisting of durvalumab, avelumab, and atezolizumab,

thereby treating the squamous-cell bladder cancer in the patient. In some aspects, the squamous-cell bladder cancer is characterized by low or negative PD-L1 expression.

In some aspects, the disclosure provides a method for treating bladder cancer in a human patient, comprising administering to the patient:

(a) an effective amount of an LNP encapsulated mRNA therapeutic agent comprising three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide; and

(b) an effective amount of PD-L1 antagonist selected from the group consisting of durvalumab, avelumab, and atezolizumab,

wherein the patient is administered a dose of0.5 mg of the mRNA therapeutic agent,

thereby treating the bladder cancer in the patient.

In some aspects, the disclosure provides a method for treating bladder cancer in a human patient, comprising administering to the patient:

(a) an effective amount of an LNP encapsulated mRNA therapeutic agent comprising three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide; and

(b) an effective amount of PD-L1 antagonist selected from the group consisting of durvalumab, avelumab, and atezolizumab,

wherein the patient is administered a dose of 0.5 mg of the mRNA therapeutic agent, wherein the dose is administered in a first dosing cycle of every 2 weeks and at least two subsequent dosing cycles of every 4 weeks,

thereby treating the bladder cancer in the patient.

In any of the foregoing or related aspects, the bladder cancer is a urothelial cancer. In any of the foregoing or related aspects, the bladder cancer is a squamous-cell bladder cancer. In any of the foregoing or related aspects, the bladder cancer is a bladder adenocarcinoma.

In some aspects, the disclosure provides a method for treating non-small cell lung carcinoma (NSCLC) in a human patient, comprising administering to the patient:

(a) an effective amount of an LNP encapsulated mRNA therapeutic agent comprising three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide; and

(b) an effective amount of PD-L1 antagonist selected from the group consisting of durvalumab, avelumab, and atezolizumab,

thereby treating the NSCLC in the patient.

In some aspects, the disclosure provides a method for treating non-small cell lung carcinoma (NSCLC) refractory to immune checkpoint inhibitor therapy in a human patient, comprising administering to the patient:

(a) an effective amount of an LNP encapsulated mRNA therapeutic agent comprising three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide; and

(b) an effective amount of PD-L1 antagonist selected from the group consisting of durvalumab, avelumab, and atezolizumab,

thereby treating the NSCLC in the patient. In some aspects, the NSCLC is primary refractory to immune checkpoint inhibitory therapy. In some aspects, the NSCLC has acquired secondary resistance to immune checkpoint inhibitor therapy.

In some aspects, the disclosure provides a method for treating melanoma refractory to immune checkpoint inhibitor therapy in a human patient, comprising administering to the patient:

(a) an effective amount of an LNP encapsulated mRNA therapeutic agent comprising three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide; and

(b) an effective amount of PD-L1 antagonist selected from the group consisting of durvalumab, avelumab, and atezolizumab,

thereby treating the melanoma in the patient. In some aspects, the melanoma is primary refractory to immune checkpoint inhibitory therapy. In some aspects, the melanoma has acquired secondary resistance to immune checkpoint inhibitor therapy.

In some aspects, the disclosure provides a method for treating melanoma in a human patient, comprising administering to the patient:

(a) an effective amount of an LNP encapsulated mRNA therapeutic agent comprising three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide; and

(b) an effective amount of PD-L1 antagonist selected from the group consisting of durvalumab, avelumab, and atezolizumab,

wherein the patient has not received anti-cancer treatment prior to administering the mRNA therapeutic, thereby treating the melanoma in the patient.

In some aspects, the disclosure provides a method for treating melanoma in a human patient, comprising administering to the patient:

(a) an effective amount of an LNP encapsulated mRNA therapeutic agent comprising three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide; and

(b) an effective amount of CTLA-4 antagonist selected from the group consisting of selected from the group consisting of ipilimumab and tremelimumab,

thereby treating the melanoma in the patient.

In any of the foregoing or related aspects of the methods of the present disclosure, the patient is administered a dose of the mRNA therapeutic (i) in a first dosing cycle comprising a dosing interval of the dose once a week for a duration of time, and at least one subsequent dosing cycle comprising a dosing interval of the dose once a week, once every 2 weeks, once every 3 weeks or once every 4 weeks, for a duration of time; (ii) in a first dosing cycle comprising a dosing interval of the dose once every 2 weeks for a duration of time, and at least one subsequent dosing cycle comprising a dosing interval of the dose once every 4 weeks for a duration of time; or (iii) in a first dosing cycle comprising a dosing interval of the dose once every 2 weeks for a duration of time, and up to six subsequent dosing cycles each comprising a dosing interval of the dose once every 4 weeks for a duration of time.

In any of the foregoing or related aspects of the methods of the present disclosure, the patient is administered a dose of the mRNA therapeutic (i) in a first dosing cycle comprising a dosing interval of the dose once a week for 28-42 days, and at least one subsequent dosing cycle comprising a dosing interval of the dose once a week, once every 2 weeks, once every 3 weeks or once every 4 weeks, for 28-42 days; (ii) in a first dosing cycle comprising a dosing interval of the dose once every 2 weeks for 28-42 days, and at least one subsequent dosing cycle comprising a dosing interval of the dose once every 4 weeks for 28-42 days or (iii) in a first dosing cycle comprising a dosing interval of the dose once every 2 weeks for 28-42 days, and up to six subsequent dosing cycles each comprising a dosing interval of the dose once every 4 weeks for 28-42 days.

In any of the foregoing or related aspects of the methods of the present disclosure, the patient is administered a dose of the mRNA therapeutic (i) in a first dosing cycle comprising a dosing interval of the dose once weekly for 28 days, and at least one subsequent dosing cycle comprising a dosing interval of the dose once every 1-4 weeks for 28 days; (ii) in a first dosing cycle comprising a dosing interval of the dose once every 2 weeks for 28 days, and at least one subsequent dosing cycle comprising a dosing interval of the dose once every 4 weeks for 28 days or (iii) in a first dosing cycle comprising a dosing interval of the dose once every 2 weeks for 28 days, and up to six subsequent dosing cycles each comprising a dosing interval of the dose once every 4 weeks for 28 days.

In any of the foregoing or related aspects of the methods of the present disclosure, an anti-tumor immune response is induced or enhanced in the patient.

In any of the foregoing or related aspects of the methods of the present disclosure, the mRNA therapeutic agent is administered to the patient by intratumoral injection.

In any of the foregoing or related aspects of the methods of the present disclosure, the PD-L1 antagonist or CTLA-4 antagonist is administered to the patient by intravenous injection.

In any of the foregoing or related aspects of the methods of the present disclosure, the PD-L1 antagonist is durvalumab. In some aspects, the patient is administered a dose of durvalumab of 1500 mg.

In any of the foregoing or related aspects of the methods of the present disclosure, the CTLA-4 antagonist is tremelimumab. In some aspects, the patient is administered a dose of tremelimumab of 225 mg.

In any of the foregoing or related aspects of the methods of the present disclosure, the patient is administered a dose of the mRNA therapeutic agent selected from 0.25 mg, 0.5 mg, 1 mg, 2 mg, 4 mg, and 8 mg.

In any of the foregoing or related aspects of the methods of the present disclosure, the patient is administered a dose of the mRNA therapeutic agent every 4 weeks.

In any of the foregoing or related aspects of the methods of the present disclosure, the patient is administered a dose of the PD-L1 antagonist or CTLA-4 antagonist every 4 weeks.

In any of the foregoing or related aspects of the methods of the present disclosure, the mRNA therapeutic agent and the PD-L1 antagonist or the CTLA-4 antagonist are administered to the patient in a dosing regimen selected from 7 to 28 days, 7 to 21 days, 7 to 14 days, 28 days, 21 days, 14 days, and 7 days.

In any of the foregoing or related aspects of the methods of the present disclosure, the mRNA therapeutic agent and the PD-L1 antagonist or the CTLA-4 antagonist are administered to the patient in a dosing regimen of 28 days.

In any of the foregoing or related aspects of the methods of the present disclosure, the patient is administered a dose of the mRNA therapeutic agent prior to administration of the PD-L1 antagonist or the CTLA-4 antagonist.

In any of the foregoing or related aspects of the methods of the present disclosure, the human OX40L polypeptide comprises the amino acid sequence as set forth in SEQ ID NO: 1; the human IL-23 polypeptide comprises the amino acid sequence as set forth in SEQ ID NO: 24; and the human IL-36γ polypeptide comprises the amino acid sequence as set forth in SEQ ID NO: 27.

In any of the foregoing or related aspects of the methods of the present disclosure, the LNP encapsulated mRNA therapeutic agent comprises three mRNA drug substances, wherein

(i) the first mRNA encoding a human OX40L polypeptide comprises an ORF comprising a nucleotide sequence at least 80% identical to the nucleotide sequence set forth in SEQ ID NO: 4 or comprises the nucleotide sequence set forth in SEQ ID NO: 4;

(ii) the second mRNA encoding a human IL-23 polypeptide comprises an ORF comprising a nucleotide sequence at least 80% identical to the nucleotide sequence set forth in SEQ ID NO: 25 or comprises the nucleotide sequence set forth in SEQ ID NO: 25; and

(iii) the third mRNA encoding a human IL-36γ polypeptide comprises an ORF comprising a nucleotide sequence at least 80% identical to the nucleotide sequence set forth in SEQ ID NO: 28 or comprises the nucleotide sequence set forth in SEQ ID NO: 28.

In any of the foregoing or related aspects of the methods of the present disclosure, each of the first mRNA, second mRNA, and third mRNA comprise a 3′ untranslated region (UTR) comprising at least one microRNA-122 (miR-122) binding site. In some aspects, the miR-122 binding site is a miR-122-3p binding site or a miR-122-5p binding site. In some aspects, the miR-122-5p binding site comprises the nucleotide sequence set forth in SEQ ID NO: 20. In some aspects, the 3′UTR comprises a nucleotide sequence at least 90% identical to the sequence set forth in SEQ ID NO: 17 or comprises the nucleotide sequence as set forth in SEQ ID NO: 17. In some aspects, each of the first, second, and third mRNAs comprise a 5′cap, a 5′ untranslated region (UTR), and a poly-A tail of about 100 nucleotides in length. In some aspects, the 5′UTR comprises a nucleotide sequence at least 90% identical to the sequence set forth in SEQ ID NO: 15 or comprises the nucleotide sequence as set forth in SEQ ID NO: 15. In some aspects, the 5′UTR comprises a nucleotide sequence at least 90% identical to the sequence set forth in SEQ ID NO: 16 or comprises the nucleotide sequence as set forth in SEQ ID NO: 16.

In any of the foregoing or related aspects of the methods of the present disclosure, the LNP encapsulated mRNA therapeutic agent comprises three mRNA drug substances, wherein

(i) the first mRNA encoding the human OX40L polypeptide comprises a nucleotide sequence at least 90% identical to the nucleotide sequence set forth in SEQ ID NO: 5 or comprises the nucleotide sequence set forth in SEQ ID NO: 5;

(ii) the second mRNA encoding a human IL-23 polypeptide comprises a nucleotide sequence at least 90% identical to the nucleotide sequence set forth in SEQ ID NO: 26 or comprises the nucleotide sequence set forth in SEQ ID NO: 26; and

(iii) the third mRNA encoding a human IL-36γ polypeptide comprises a nucleotide sequence at least 90% identical to the nucleotide sequence set forth in SEQ ID NO: 29 or comprises the nucleotide sequence set forth in SEQ ID NO: 29.

In some aspects, the disclosure provides a method for treating advanced or metastatic solid tumor malignancy or lymphoma in a human patient by inducing or enhancing an anti-tumor immune response, comprising administering to the patient by intratumoral injection an effective amount of an LNP encapsulated mRNA therapeutic agent comprising three mRNA drug substances:

(i) a first mRNA encoding a human OX40L polypeptide comprising an ORF comprising a nucleotide sequence at least 80% identical to the nucleotide sequence set forth in SEQ ID NO: 4 or comprises the nucleotide sequence set forth in SEQ ID NO: 4;

(ii) a second mRNA encoding a human IL-23 polypeptide comprising an ORF comprising a nucleotide sequence at least 80% identical to the nucleotide sequence set forth in SEQ ID NO: 25 or comprises the nucleotide sequence set forth in SEQ ID NO: 25; and

(iii) a third mRNA encoding a human IL-36γ polypeptide comprising an ORF comprising a nucleotide sequence at least 80% identical to the nucleotide sequence set forth in SEQ ID NO: 28 or comprises the nucleotide sequence set forth in SEQ ID NO: 28, wherein the patient is administered a dose of the mRNA therapeutic agent selected from 0.25 mg, 0.5 mg, 1 mg, 2 mg, 4 mg, and 8 mg, thereby treating the advanced or metastatic solid tumor malignancy or lymphoma in the patient by inducing or enhancing an anti-tumor immune response. In some aspects, the patient is administered a dose of 0.25 mg of the mRNA therapeutic agent. In some aspects, the patient is administered a dose of 0.50 mg of the mRNA therapeutic agent. In some aspects, the patient is administered a dose of 1.0 mg of the mRNA therapeutic agent. In some aspects, the patient is administered a dose of 2.0 mg of the mRNA therapeutic agent. In some aspects, the patient is administered a dose of 4.0 mg of the mRNA therapeutic agent. In some aspects, the patient is administered a dose of 8.0 mg of the mRNA therapeutic agent. In some aspects, the patient is administered a dose of the mRNA therapeutic agent every 2 weeks. In some aspects, the patient is administered a dose of the mRNA therapeutic agent every 3 weeks. In some aspects, the patient is administered a dose of the mRNA therapeutic agent every 4 weeks.

In some aspects, the disclosure provides a method for treating advanced or metastatic solid tumor malignancy or lymphoma in a human patient by inducing or enhancing an anti-tumor immune response, comprising administering to the patient by intratumoral injection an effective amount of an LNP encapsulated mRNA therapeutic agent comprising three mRNA drug substances:

(i) a first mRNA encoding the human OX40L polypeptide comprising a nucleotide sequence at least 90% identical to the nucleotide sequence set forth in SEQ ID NO: 5 or comprising the nucleotide sequence set forth in SEQ ID NO: 5;

(ii) a second mRNA encoding a human IL-23 polypeptide comprising a nucleotide sequence at least 90% identical to the nucleotide sequence set forth in SEQ ID NO: 26 or comprising the nucleotide sequence set forth in SEQ ID NO: 26; and

(iii) a third mRNA encoding a human IL-36γ polypeptide comprising a nucleotide sequence at least 90% identical to the nucleotide sequence set forth in SEQ ID NO: 29 or comprising the nucleotide sequence set forth in SEQ ID NO: 29, wherein the patient is administered a dose of the mRNA therapeutic agent selected from 0.25 mg, 0.5 mg, 1 mg, 2 mg, 4 mg, and 8 mg, thereby treating the advanced or metastatic solid tumor malignancy or lymphoma in the patient by inducing or enhancing an anti-tumor immune response.

In some aspects, the disclosure provides a method for treating advanced or metastatic solid tumor malignancy or lymphoma in a human patient, comprising administering to the patient, optionally by intratumoral injection, an effective amount of an LNP encapsulated mRNA therapeutic agent comprising three mRNA drug substances:

(i) a first mRNA encoding the human OX40L polypeptide comprising a nucleotide sequence at least 90% identical to the nucleotide sequence set forth in SEQ ID NO: 5 or comprising the nucleotide sequence set forth in SEQ ID NO: 5;

(ii) a second mRNA encoding a human IL-23 polypeptide comprising a nucleotide sequence at least 90% identical to the nucleotide sequence set forth in SEQ ID NO: 26 or comprising the nucleotide sequence set forth in SEQ ID NO: 26; and

(iii) a third mRNA encoding a human IL-36γ polypeptide comprising a nucleotide sequence at least 90% identical to the nucleotide sequence set forth in SEQ ID NO: 29 or comprising the nucleotide sequence set forth in SEQ ID NO: 29, wherein the patient is administered a dose of the mRNA therapeutic agent selected from 0.25 mg, 0.5 mg, 1 mg, 2 mg, 4 mg, and 8 mg, thereby treating the advanced or metastatic solid tumor malignancy or lymphoma in the patient.

In some aspects, the disclosure provides a method for treating advanced or metastatic solid tumor malignancy or lymphoma in a human patient, comprising administering to the patient, an effective amount of an LNP encapsulated mRNA therapeutic agent comprising three mRNA drug substances:

(i) a first mRNA encoding the human OX40L polypeptide comprising a nucleotide sequence at least 90% identical to the nucleotide sequence set forth in SEQ ID NO: 5 or comprising the nucleotide sequence set forth in SEQ ID NO: 5;

(ii) a second mRNA encoding a human IL-23 polypeptide comprising a nucleotide sequence at least 90% identical to the nucleotide sequence set forth in SEQ ID NO: 26 or comprising the nucleotide sequence set forth in SEQ ID NO: 26; and

(iii) a third mRNA encoding a human IL-36γ polypeptide comprising a nucleotide sequence at least 90% identical to the nucleotide sequence set forth in SEQ ID NO: 29 or comprising the nucleotide sequence set forth in SEQ ID NO: 29,

wherein the patient is administered a dose of 0.10-10.0 mg of the mRNA therapeutic agent in a dosing regimen comprising a first dosing cycle and at least one subsequent dosing cycle, wherein the first and subsequent dosing cycles comprise administration of the mRNA therapeutic at a dosing interval selected from once a week, once every 2 weeks, once every 3 weeks, and once every 4 weeks for a duration of time, wherein the dosing intervals for the first and subsequent dosing cycles are different, thereby treating the advanced or metastatic solid tumor malignancy or lymphoma in the patient.

In some aspects, the disclosure provides a method for treating advanced or metastatic solid tumor malignancy or lymphoma in a human patient, comprising administering to the patient, an effective amount of an LNP encapsulated mRNA therapeutic agent comprising three mRNA drug substances:

(i) a first mRNA encoding the human OX40L polypeptide comprising a nucleotide sequence at least 90% identical to the nucleotide sequence set forth in SEQ ID NO: 5 or comprising the nucleotide sequence set forth in SEQ ID NO: 5;

(ii) a second mRNA encoding a human IL-23 polypeptide comprising a nucleotide sequence at least 90% identical to the nucleotide sequence set forth in SEQ ID NO: 26 or comprising the nucleotide sequence set forth in SEQ ID NO: 26; and

(iii) a third mRNA encoding a human IL-36γ polypeptide comprising a nucleotide sequence at least 90% identical to the nucleotide sequence set forth in SEQ ID NO: 29 or comprising the nucleotide sequence set forth in SEQ ID NO: 29,

wherein the patient is administered a dose of 0.10-10.0 mg of the mRNA therapeutic agent, wherein the solid tumor malignancy is selected from the group consisting of: a bladder cancer, an immune checkpoint inhibitor (CPI)-refractory melanoma, a neoadjuvant melanoma, and a non-small cell lung carcinoma, thereby treating the advanced or metastatic solid tumor malignancy or lymphoma in the patient.

In some aspects, the patient is administered a dose of 0.25 mg of the mRNA therapeutic agent. In some aspects, the patient is administered a dose of 0.50 mg of the mRNA therapeutic agent. In some aspects, the patient is administered a dose of 1.0 mg of the mRNA therapeutic agent. In some aspects, the patient is administered a dose of 2.0 mg of the mRNA therapeutic agent. In some aspects, the patient is administered a dose of 4.0 mg of the mRNA therapeutic agent. In some aspects, the patient is administered a dose of 8.0 mg of the mRNA therapeutic agent. In some aspects, the patient is administered a dose of the mRNA therapeutic agent every 2 weeks. In some aspects, the patient is administered a dose of the mRNA therapeutic agent every 3 weeks. In some aspects, the patient is administered a dose of the mRNA therapeutic agent every 4 weeks.

In any of the foregoing or related aspects of the methods of the present disclosure, the LNP encapsulated mRNA therapeutic agent comprises three mRNA drug substances, wherein the first, second, and third mRNAs are formulated in the lipid nanoparticle at a mass ratio of OX40L:IL-23:IL-36γ of 1:1:2.

In any of the foregoing or related aspects of the methods of the present disclosure, the LNP encapsulated mRNA therapeutic agent comprises three mRNA drug substances, wherein each of the first, second and third mRNAs is chemically modified.

In any of the foregoing or related aspects of the methods of the present disclosure, the LNP encapsulated mRNA therapeutic agent comprises three mRNA drug substances, wherein each of the first, second, and third mRNAs is fully modified with chemically-modified uridines. In some aspects, the chemically-modified uridines are N1-methylpseudouridines (m1ψ). In some aspects, each of the first, second and third mRNAs is fully modified with 5-methylcytosine or is fully modified with N1-methylpseudouridines (m1ψ) and 5-methylcytosine.

In any of the foregoing or related aspects of the methods of the present disclosure, the LNP encapsulated mRNA therapeutic agent comprises three mRNA drug substances, wherein the LNP comprises a compound having the formula:

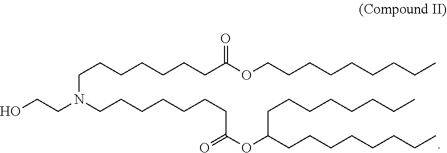 [SMILES: CCCCCCCCCOC(=O)CCCCCCCN(CCO)CCCCCCCC(=O)OC(CCCCCCCC)CCCCCCCC]

In some aspects, the LNP further comprising a phospholipid, a structural lipid, and a PEG lipid. In some aspects, the LNP comprises a molar ratio of about 20-60% ionizable amino lipid, about 5-25% phospholipid, about 25-55% structural lipid, and about 0.5-1.5% PEG lipid. In some aspects, the LNP comprises a molar ratio of about 50% ionizable amino lipid, about 10% phospholipid, about 38.5% structural lipid, and about 1.5% PEG lipid. In some aspects, the LNP comprises a molar ratio of about 50% ionizable amino lipid, about 10% phospholipid, about 38.5% cholesterol, and about 1.5% PEG-DMG.

In any of the foregoing or related aspects, the LNP comprises a molar ratio of: (1) 40-60% ionizable amino lipid, 8-16% phospholipid, 30-45% sterol, and 1-5% PEG modified lipid; or (2) 45-65% ionizable amino lipid, 5-10% phospholipid, 25-40% sterol, and 0.5-5% PEG modified lipid. In some aspects, the LNP comprises a molar ratio of 40-60% ionizable amino lipid, 8-16% phospholipid, 30-45% sterol, and 1-5% PEG modified lipid. In other aspects, the LNP comprises a molar ratio of 45-65% ionizable amino lipid, 5-10% phospholipid, 25-40% sterol, and 0.5-5% PEG modified lipid.

In any of the foregoing or related aspects, the LNP comprises a molar ratio of: (1) 40-60% Compound II, 8-16% DSPC, 30-45% cholesterol, and 1-5% PEG DMG; or (2) 45-65% Compound II, 5-10% DSPC, 25-40% cholesterol, and 0.5-5% PEG DMG. In some aspects, the LNP comprises a molar ratio of 40-60% Compound II, 8-16% DSPC, 30-45% cholesterol, and 1-5% PEG DMG. In some aspects, the LNP comprises a molar ratio of 45-65% Compound II, 5-10% DSPC, 25-40% cholesterol, and 0.5-5% PEG DMG.

In any of the foregoing or related aspects of the methods of the present disclosure, the LNP encapsulated mRNA therapeutic agent comprises three mRNA drug substances, wherein the mRNA therapeutic agent is administered by a single injection. In some aspects, the mRNA therapeutic agent is administered by multiple injections into one or more different sites within the same tumor lesion or divided across several tumor lesions.

In any of the foregoing or related aspects of the methods of the present disclosure, the LNP encapsulated mRNA therapeutic agent comprises three mRNA drug substances, wherein the LNP is formulated in a pharmaceutically acceptable carrier. In some aspects, the pharmaceutically acceptable carrier is a solution suitable for intratumoral injection. In some aspects, the solution comprises a buffer.

In any of the foregoing or related aspects of the methods of the present disclosure, treatment results in an anti-tumor immune response in the patient comprising T cell activation, T cell proliferation, and/or T cell expansion. In some aspects, the T cells are CD4+ T cells, CD8+ T cells, or both CD4+ T cells and CD8+ T cells. In some aspects, the anti-tumor immune response (e.g., T cell proliferation) persists for at least 1 week, 2 weeks, 3 weeks or 4 weeks after treatment. In some aspects, treatment results in a reduction in size or inhibition of growth of the injected tumor. In some aspects, treatment results in a reduction in size or inhibition of growth of an uninjected tumor. In some aspects, treatment results in an increase in expression of IL-23 and/or IL-36γ in the plasma and/or tumor of the patient. In some aspects, treatment results in an increase in expression of IL-22, IL-6, TNFα, IFNγ and any combination thereof in the plasma and/or tumor of the patient. In some aspects, increased expression occurs 3 hours, 6 hours and/or 24 hours after treatment. In some aspects, increased expression persists for at least 24 hours, 48 hours, 72 hours or 96 hours. In some aspects, increased expression persists for at least 7 days. In some aspects, increased expression occurs 24-48 hours, 24-72 hours or 24-96 hours after treatment. In some aspects, treatment results in an increase in PD-L1 expression in tumor cells and/or immune cells within the tumor microenvironment. In some aspects, PD-L1 expression is increased 24 hours after treatment. In some aspects, increased PD-L1 expression persists for at least 1 week, 2 weeks, or 3 weeks. In some aspects, PD-L1 expression correlates with IFNγ expression. In some aspects, PD-L1 expression correlates with IFNγ expression before treatment. In some aspects, PD-L1 expression correlates with IFNγ expression at least 24 hours after treatment, at least 1 week after treatment, at least 2 weeks after treatment, or at least 3 weeks after treatment. In some aspects, expression is determined by measuring protein levels.

In other aspects, the disclosure provides an LNP encapsulated mRNA therapeutic agent for use in the manufacture of a medicament for treating advanced or metastatic solid tumor malignancy or lymphoma in a human patient by inducing or enhancing an anti-tumor immune response, wherein the medicament comprises the LNP encapsulated mRNA therapeutic agent, and an optional pharmaceutically acceptable carrier, and wherein the treatment comprises administration of the medicament by intratumoral injection at a dose of 0.10-10.0 mg of the mRNA therapeutic agent in a dosing regimen from 7 to 28 days, and wherein the LNP encapsulated mRNA therapeutic agent comprises three mRNA drug substances:

(i) a first mRNA comprising an ORF encoding a human OX40L polypeptide;

(ii) a second mRNA comprising an ORF encoding a human IL-23 polypeptide; and

(iii) a third mRNA comprising an ORF encoding a human IL-36γ polypeptide.

In some aspects, the disclosure provides an LNP encapsulated mRNA therapeutic agent for use in the manufacture of a medicament for treating advanced or metastatic solid tumor malignancy or lymphoma in a human patient, wherein the medicament comprises the LNP encapsulated mRNA therapeutic agent, and an optional pharmaceutically acceptable carrier, and wherein the treatment comprises administration of the medicament, optionally by intratumoral injection, at a dose of 0.10-10.0 mg of the mRNA therapeutic agent in a dosing regimen from 7 to 28 days, and wherein the LNP encapsulated mRNA therapeutic agent comprises three mRNA drug substances:

(i) a first mRNA comprising an ORF encoding a human OX40L polypeptide;

(ii) a second mRNA comprising an ORF encoding a human IL-23 polypeptide; and

(iii) a third mRNA comprising an ORF encoding a human IL-36γ polypeptide.

In other aspects, the disclosure provides an LNP encapsulated mRNA therapeutic agent for use in the manufacture of a medicament for treating advanced or metastatic solid tumor malignancy or lymphoma in a human patient by inducing or enhancing an anti-tumor immune response, wherein the medicament comprises the LNP encapsulated mRNA therapeutic agent, and an optional pharmaceutically acceptable carrier, and wherein the treatment comprises administration of the medicament by intratumoral injection at a dose of 0.10-10.0 mg of the mRNA therapeutic agent in a dosing regimen comprising a first dosing cycle and at least one subsequent dosing cycle, wherein the first and subsequent dosing cycles comprise administration of the mRNA therapeutic at a dosing interval selected from once a week, once every 2 weeks, once every 3 weeks, and once every 4 weeks for a duration of time, wherein the dosing intervals for the first and subsequent dosing cycles are different, and wherein the LNP encapsulated mRNA therapeutic agent comprises three mRNA drug substances:

(i) a first mRNA comprising an ORF encoding a human OX40L polypeptide;

(ii) a second mRNA comprising an ORF encoding a human IL-23 polypeptide; and

(iii) a third mRNA comprising an ORF encoding a human IL-36γ polypeptide.

In yet further aspects, the disclosure provides an LNP encapsulated mRNA therapeutic agent for use in the manufacture of a medicament for treating advanced or metastatic solid tumor malignancy or lymphoma in a human patient by inducing or enhancing an anti-tumor immune response, wherein the medicament comprises the LNP encapsulated mRNA therapeutic agent, and an optional pharmaceutically acceptable carrier, and wherein the treatment comprises administration of the medicament by intratumoral injection at a dose of 0.10-10.0 mg of the mRNA therapeutic agent, wherein the solid tumor malignancy is selected from the group consisting of: a bladder cancer, an immune checkpoint inhibitor (CPI)-refractory melanoma, a neoadjuvant melanoma, and a non-small cell lung carcinoma, and wherein the LNP encapsulated mRNA therapeutic agent comprises three mRNA drug substances:

(i) a first mRNA comprising an ORF encoding a human OX40L polypeptide;

(ii) a second mRNA comprising an ORF encoding a human IL-23 polypeptide; and

(iii) a third mRNA comprising an ORF encoding a human IL-36γ polypeptide.

In other aspects, the disclosure provides a method for treating advanced or metastatic solid tumor malignancy or lymphoma in a human patient, comprising administering an effective amount of a PD-1 antagonist, a PD-L1 antagonist or a CTLA-4 antagonist, to a patient that has received or is receiving the LNP encapsulated mRNA therapeutic described herein, thereby treating the patient.

In yet other aspects, the disclosure provides a kit comprising a container comprising a pharmaceutical composition comprising: an LNP encapsulated mRNA therapeutic agent; and a pharmaceutically acceptable carrier, and instructions for use in treating advanced or metastatic solid tumor malignancy or lymphoma in a human patient by inducing or enhancing an anti-tumor immune response, wherein the treatment comprises administration of the pharmaceutical composition by intratumoral injection at a dose of 0.10-10.0 mg of the mRNA therapeutic agent in a dosing regimen from 7 to 28 days, and wherein the LNP encapsulated mRNA therapeutic agent comprises three mRNA drug substances:

(i) a first mRNA comprising an ORF encoding a human OX40L polypeptide;

(ii) a second mRNA comprising an ORF encoding a human IL-23 polypeptide; and

(iii) a third mRNA comprising an ORF encoding a human IL-36γ polypeptide.

In some aspects, the disclosure provides a kit comprising a container comprising a pharmaceutical composition comprising: an LNP encapsulated mRNA therapeutic agent; and a pharmaceutically acceptable carrier, and instructions for use in treating advanced or metastatic solid tumor malignancy or lymphoma in a human patient, wherein the treatment comprises administration of the pharmaceutical composition, optionally by intratumoral injection, at a dose of 0.10-10.0 mg of the mRNA therapeutic agent in a dosing regimen from 7 to 28 days, and wherein the LNP encapsulated mRNA therapeutic agent comprises three mRNA drug substances:

(i) a first mRNA comprising an ORF encoding a human OX40L polypeptide;

(ii) a second mRNA comprising an ORF encoding a human IL-23 polypeptide; and

(iii) a third mRNA comprising an ORF encoding a human IL-36γ polypeptide.

In other aspects, the disclosure provides a kit comprising a container comprising a pharmaceutical composition comprising: an LNP encapsulated mRNA therapeutic agent; and a pharmaceutically acceptable carrier, and instructions for use in treating advanced or metastatic solid tumor malignancy or lymphoma in a human patient, wherein the treatment comprises administration of the pharmaceutical composition by intratumoral injection at a dose of 0.10-10.0 mg of the mRNA therapeutic agent in a dosing regimen comprising a first dosing cycle and at least one subsequent dosing cycle, wherein the first and subsequent dosing cycles comprise administration of the mRNA therapeutic at a dosing interval selected from once a week, once every 2 weeks, once every 3 weeks, and once every 4 weeks for a duration of time, wherein the dosing intervals for the first and subsequent dosing cycles are different, and wherein the LNP encapsulated mRNA therapeutic agent comprises three mRNA drug substances:

(i) a first mRNA comprising an ORF encoding a human OX40L polypeptide;

(ii) a second mRNA comprising an ORF encoding a human IL-23 polypeptide; and

(iii) a third mRNA comprising an ORF encoding a human IL-36γ polypeptide.

In further aspects, the disclosure provides a kit comprising a container comprising a pharmaceutical composition comprising: an LNP encapsulated mRNA therapeutic agent; and a pharmaceutically acceptable carrier, and instructions for use in treating advanced or metastatic solid tumor malignancy in a human patient, wherein the treatment comprises administration of the pharmaceutical composition by intratumoral injection at a dose of 0.10-10.0 mg of the mRNA therapeutic agent, wherein the solid tumor malignancy is selected from the group consisting of: a bladder cancer, an immune checkpoint inhibitor (CPI)-refractory melanoma, a neoadjuvant melanoma, and a non-small cell lung carcinoma, and wherein the LNP encapsulated mRNA therapeutic agent comprises three mRNA drug substances;

(i) a first mRNA comprising an ORF encoding a human OX40L polypeptide;

(ii) a second mRNA comprising an ORF encoding a human IL-23 polypeptide; and

(iii) a third mRNA comprising an ORF encoding a human IL-36γ polypeptide.

In some aspects, the disclosure provides a kit comprising a container comprising a pharmaceutical composition comprising: an LNP encapsulated mRNA therapeutic agent; and a pharmaceutically acceptable carrier, wherein the pharmaceutical composition comprises about 2 mg/mL of the mRNA therapeutic agent, and instructions for use in treating advanced or metastatic solid tumor malignancy or lymphoma in a human patient, wherein the treatment comprises administration of the pharmaceutical composition, optionally by intratumoral injection, at a dose of 0.10-10.0 mg of the mRNA therapeutic agent in a dosing regimen from 7 to 28 days, and wherein the LNP encapsulated mRNA therapeutic agent comprises three mRNA drug substances:

(i) a first mRNA comprising an ORF encoding a human OX40L polypeptide;

(ii) a second mRNA comprising an ORF encoding a human IL-23 polypeptide; and

(iii) a third mRNA comprising an ORF encoding a human IL-36γ polypeptide.

In other aspects, the disclosure provides a kit comprising a container comprising a pharmaceutical composition comprising: an LNP encapsulated mRNA therapeutic agent; and a pharmaceutically acceptable carrier, wherein the pharmaceutical composition comprises about 2 mg/mL of the mRNA therapeutic agent, and instructions for use in treating advanced or metastatic solid tumor malignancy or lymphoma in a human patient, wherein the treatment comprises administration of the pharmaceutical composition, optionally by intratumoral injection, at a dose of 0.10-10.0 mg of the mRNA therapeutic agent in a dosing regimen comprising a first dosing cycle and at least one subsequent dosing cycle, wherein the first and subsequent dosing cycles comprise administration of the mRNA therapeutic at a dosing interval selected from once a week, once every 2 weeks, once every 3 weeks, and once every 4 weeks for a duration of time, wherein the dosing intervals for the first and subsequent dosing cycles are different, and wherein the LNP encapsulated mRNA therapeutic agent comprises three mRNA drug substances:

(i) a first mRNA comprising an ORF encoding a human OX40L polypeptide;

(ii) a second mRNA comprising an ORF encoding a human IL-23 polypeptide; and

(iii) a third mRNA comprising an ORF encoding a human IL-36γ polypeptide.

In further aspects, the disclosure provides a kit comprising a container comprising a pharmaceutical composition comprising: an LNP encapsulated mRNA therapeutic agent; and a pharmaceutically acceptable carrier, wherein the pharmaceutical composition comprises about 2 mg/mL of the mRNA therapeutic agent, and instructions for use in treating advanced or metastatic solid tumor malignancy in a human patient, wherein the treatment comprises administration of the pharmaceutical composition by intratumoral injection at a dose of 0.10-10.0 mg of the mRNA therapeutic agent, wherein the solid tumor malignancy is selected from the group consisting of: a bladder cancer, an immune checkpoint inhibitor (CPI)-refractory melanoma, a neoadjuvant melanoma, and a non-small cell lung carcinoma, and wherein the LNP encapsulated mRNA therapeutic agent comprises three mRNA drug substances:

(i) a first mRNA comprising an ORF encoding a human OX40L polypeptide;

(ii) a second mRNA comprising an ORF encoding a human IL-23 polypeptide; and

(iii) a third mRNA comprising an ORF encoding a human IL-36γ polypeptide.

In any of the foregoing or related aspects of the use or kits of the present disclosure, treatment comprises administration of the medicament at a dose of the mRNA therapeutic agent selected from: 0.25-8.0 mg; 0.25-4.0 mg; 0.25-2.0 mg; 0.25-1.0 mg; 0.25-5 mg; 0.5-8.0 mg; 0.5-4.0 mg; 0.5-2.0 mg; 0.5-1.0 mg; 1.0-8.0 mg; 1.0-4.0 mg; 1.0-2.0 mg; 2.0-8.0 mg; 2.0-4.0 mg; and 4.0-8.0 mg.

In any of the foregoing or related aspects of the use or kits of the present disclosure, treatment comprises administration of the medicament at a dose of the mRNA therapeutic agent selected from 0.10 mg, 0.25 mg, 0.50 mg, 1.0 mg, 2.0 mg, 4.0 mg, 8.0 mg and 10.0 mg.

In any of the foregoing or related aspects of the use or kits of the present disclosure, treatment comprises administration of the medicament to the patient in a dosing regimen selected from 7 to 21 days, 7 to 14 days, 28 days, 21 days, 14 days, and 7 days.

In any of the foregoing or related aspects of the use or kits of the present disclosure, treatment comprises administration of the medicament every 2 weeks. In any of the foregoing or related aspects of the use or kits of the present disclosure, treatment comprises administration of the medicament every 3 weeks. In any of the foregoing or related aspects of the use or kits of the present disclosure, treatment comprises administration of the medicament every 4 weeks.

In any of the foregoing or related aspects of the use or kits of the present disclosure, treatment comprises administration of the medicament or pharmaceutical composition in combination with a composition comprising a PD-1 antagonist, a PD-L1 antagonist, or a CTLA-4 antagonist, and an optional pharmaceutically acceptable carrier.

In any of the foregoing or related aspects of the use or kits of the present disclosure, the PD-1 antagonist is an antibody or antigen binding portion thereof that specifically binds to PD-1. In some aspects, the PD-1 antagonist is selected from the group consisting of nivolumab, pembrolizumab, and pidilizumab.

In any of the foregoing or related aspects of the use or kits of the present disclosure, the PD-L1 antagonist is an antibody or antigen binding portion thereof that specifically binds to PD-L1. In some aspects, the PD-L1 antagonist is selected from the group consisting of durvalumab, avelumab, and atezolizumab. In some aspects, the PD-L1 antagonist is durvalumab.

In any of the foregoing or related aspects of the use or kits of the present disclosure, the CTLA-4 antagonist is an antibody or antigen binding portion thereof that specifically binds to CTLA-4. In some aspects, the CTLA-4 antagonist is selected from the group consisting of ipilimumab and tremelimumab. In some aspects, the CTLA-4 antagonist is tremelimumab.

In any of the foregoing or related aspects of the use or kits of the present disclosure, treatment comprises administration of a dose of the composition comprising the PD-1 antagonist, the PD-L1 antagonist or the CTLA-4 antagonist every 4 weeks.

In any of the foregoing or related aspects of the use or kits of the present disclosure, treatment comprises administration of the medicament comprising an LNP encapsulated mRNA therapeutic agent prior to administration of the PD-1 antagonist, PD-L1 antagonist or CTLA-4 antagonist.

In any of the foregoing or related aspects of the use or kits of the present disclosure, the advanced or metastatic solid tumor malignancy in the patient is selected from triple negative breast cancer, head and neck squamous cell carcinoma, and melanoma and the lymphoma is Non-Hodgkin lymphoma.

In any of the foregoing or related aspects of the use or kits of the present disclosure, the human OX40L polypeptide comprises the amino acid sequence as set forth in SEQ ID NO: 1; the human IL-23 polypeptide comprises the amino acid sequence as set forth in SEQ ID NO: 24; and the human IL-36γ polypeptide comprises the amino acid sequence as set forth in SEQ ID NO: 27.

In other aspects, the disclosure provides an LNP encapsulated mRNA therapeutic agent for use in the manufacture of a medicament for treating advanced or metastatic solid tumor malignancy or lymphoma in a human patient by inducing or enhancing an anti-tumor immune response, wherein the medicament comprises the LNP encapsulated mRNA therapeutic agent, and an optional pharmaceutically acceptable carrier, and wherein the treatment comprises administration of the medicament by intratumoral injection at a dose of 0.10-10.0 mg of the mRNA therapeutic agent in a dosing regimen from 7 to 28 days, and wherein the LNP encapsulated mRNA therapeutic agent comprises three mRNA drug substances:

(i) a first mRNA encoding a human OX40L polypeptide comprising an ORF comprising a nucleotide sequence at least 80% identical to the nucleotide sequence set forth in SEQ ID NO: 4 or comprises the nucleotide sequence set forth in SEQ ID NO: 4;

(ii) a second mRNA encoding a human IL-23 polypeptide comprising an ORF comprising a nucleotide sequence at least 80% identical to the nucleotide sequence set forth in SEQ ID NO: 25 or comprises the nucleotide sequence set forth in SEQ ID NO: 25; and

(iii) a third mRNA encoding a human IL-36γ polypeptide comprising an ORF comprising a nucleotide sequence at least 80% identical to the nucleotide sequence set forth in SEQ ID NO: 28 or comprises the nucleotide sequence set forth in SEQ ID NO: 28.

In other aspects, the disclosure provides an LNP encapsulated mRNA therapeutic agent for use in the manufacture of a medicament for treating advanced or metastatic solid tumor malignancy or lymphoma in a human patient, wherein the medicament comprises the LNP encapsulated mRNA therapeutic agent, and an optional pharmaceutically acceptable carrier, and wherein the treatment comprises administration of the medicament, optionally by intratumoral injection, at a dose of 0.10-10.0 mg of the mRNA therapeutic agent in a dosing regimen from 7 to 28 days, and wherein the LNP encapsulated mRNA therapeutic agent comprises three mRNA drug substances:

(i) a first mRNA encoding a human OX40L polypeptide comprising an ORF comprising a nucleotide sequence at least 80% identical to the nucleotide sequence set forth in SEQ ID NO: 4 or comprises the nucleotide sequence set forth in SEQ ID NO: 4;

(ii) a second mRNA encoding a human IL-23 polypeptide comprising an ORF comprising a nucleotide sequence at least 80% identical to the nucleotide sequence set forth in SEQ ID NO: 25 or comprises the nucleotide sequence set forth in SEQ ID NO: 25; and

(iii) a third mRNA encoding a human IL-36γ polypeptide comprising an ORF comprising a nucleotide sequence at least 80% identical to the nucleotide sequence set forth in SEQ ID NO: 28 or comprises the nucleotide sequence set forth in SEQ ID NO: 28.

In other aspects, the disclosure provides an LNP encapsulated mRNA therapeutic agent for use in the manufacture of a medicament for treating advanced or metastatic solid tumor malignancy or lymphoma in a human patient, wherein the medicament comprises the LNP encapsulated mRNA therapeutic agent, and an optional pharmaceutically acceptable carrier, and wherein the treatment comprises administration of the medicament by intratumoral injection at a dose of 0.10-10.0 mg of the mRNA therapeutic agent in a dosing regimen comprising a first dosing cycle and at least one subsequent dosing cycle, wherein the first and subsequent dosing cycles comprise administration of the mRNA therapeutic at a dosing interval selected from once a week, once every 2 weeks, once every 3 weeks, and once every 4 weeks for a duration of time, wherein the dosing intervals for the first and subsequent dosing cycles are different, and wherein the LNP encapsulated mRNA therapeutic agent comprises three mRNA drug substances:

(i) a first mRNA encoding a human OX40L polypeptide comprising an ORF comprising a nucleotide sequence at least 80% identical to the nucleotide sequence set forth in SEQ ID NO: 4 or comprises the nucleotide sequence set forth in SEQ ID NO: 4;

(ii) a second mRNA encoding a human IL-23 polypeptide comprising an ORF comprising a nucleotide sequence at least 80% identical to the nucleotide sequence set forth in SEQ ID NO: 25 or comprises the nucleotide sequence set forth in SEQ ID NO: 25; and

(iii) a third mRNA encoding a human IL-36γ polypeptide comprising an ORF comprising a nucleotide sequence at least 80% identical to the nucleotide sequence set forth in SEQ ID NO: 28 or comprises the nucleotide sequence set forth in SEQ ID NO: 28.

In other aspects, the disclosure provides an LNP encapsulated mRNA therapeutic agent for use in the manufacture of a medicament for treating advanced or metastatic solid tumor malignancy or lymphoma in a human patient, wherein the medicament comprises the LNP encapsulated mRNA therapeutic agent, and an optional pharmaceutically acceptable carrier, wherein the treatment comprises administration of the pharmaceutical composition by intratumoral injection at a dose of 0.10-10.0 mg of the mRNA therapeutic agent, wherein the solid tumor malignancy is selected from the group consisting of: a bladder cancer, an immune checkpoint inhibitor (CPI)-refractory melanoma, a neoadjuvant melanoma, and a non-small cell lung carcinoma, and wherein the LNP encapsulated mRNA therapeutic agent comprises three mRNA drug substances:

(i) a first mRNA encoding a human OX40L polypeptide comprising an ORF comprising a nucleotide sequence at least 80% identical to the nucleotide sequence set forth in SEQ ID NO: 4 or comprises the nucleotide sequence set forth in SEQ ID NO: 4;

(ii) a second mRNA encoding a human IL-23 polypeptide comprising an ORF comprising a nucleotide sequence at least 80% identical to the nucleotide sequence set forth in SEQ ID NO: 25 or comprises the nucleotide sequence set forth in SEQ ID NO: 25; and

(iii) a third mRNA encoding a human IL-36γ polypeptide comprising an ORF comprising a nucleotide sequence at least 80% identical to the nucleotide sequence set forth in SEQ ID NO: 28 or comprises the nucleotide sequence set forth in SEQ ID NO: 28.

In yet other aspects, the disclosure provides a kit comprising a container comprising a pharmaceutical composition comprising: an LNP encapsulated mRNA therapeutic agent; and a pharmaceutically acceptable carrier, and instructions for use in treating advanced or metastatic solid tumor malignancy or lymphoma in a human patient by inducing or enhancing an anti-tumor immune response, wherein the treatment comprises administration of the pharmaceutical composition by intratumoral injection at a dose of 0.10-10.0 mg of the mRNA therapeutic agent in a dosing regimen from 7 to 28 days, and wherein the LNP encapsulated mRNA therapeutic agent comprises three mRNA drug substances:

(i) a first mRNA encoding a human OX40L polypeptide comprising an ORF comprising a nucleotide sequence at least 80% identical to the nucleotide sequence set forth in SEQ ID NO: 4 or comprises the nucleotide sequence set forth in SEQ ID NO: 4;

(ii) a second mRNA encoding a human IL-23 polypeptide comprising an ORF comprising a nucleotide sequence at least 80% identical to the nucleotide sequence set forth in SEQ ID NO: 25 or comprises the nucleotide sequence set forth in SEQ ID NO: 25; and

(iii) a third mRNA encoding a human IL-36γ polypeptide comprising an ORF comprising a nucleotide sequence at least 80% identical to the nucleotide sequence set forth in SEQ ID NO: 28 or comprises the nucleotide sequence set forth in SEQ ID NO: 28.

In yet other aspects, the disclosure provides a kit comprising a container comprising a pharmaceutical composition comprising: an LNP encapsulated mRNA therapeutic agent; and a pharmaceutically acceptable carrier, and instructions for use in treating advanced or metastatic solid tumor malignancy or lymphoma in a human patient, wherein the treatment comprises administration of the pharmaceutical composition, optionally by intratumoral injection, at a dose of 0.10-10.0 mg of the mRNA therapeutic agent in a dosing regimen from 7 to 28 days, and wherein the LNP encapsulated mRNA therapeutic agent comprises three mRNA drug substances:

(i) a first mRNA encoding a human OX40L polypeptide comprising an ORF comprising a nucleotide sequence at least 80% identical to the nucleotide sequence set forth in SEQ ID NO: 4 or comprises the nucleotide sequence set forth in SEQ ID NO: 4;

(ii) a second mRNA encoding a human IL-23 polypeptide comprising an ORF comprising a nucleotide sequence at least 80% identical to the nucleotide sequence set forth in SEQ ID NO: 25 or comprises the nucleotide sequence set forth in SEQ ID NO: 25; and

(iii) a third mRNA encoding a human IL-36γ polypeptide comprising an ORF comprising a nucleotide sequence at least 80% identical to the nucleotide sequence set forth in SEQ ID NO: 28 or comprises the nucleotide sequence set forth in SEQ ID NO: 28.

In some aspects, the disclosure provides a kit comprising a container comprising a pharmaceutical composition comprising: an LNP encapsulated mRNA therapeutic agent; and a pharmaceutically acceptable carrier, and instructions for use in treating advanced or metastatic solid tumor malignancy or lymphoma in a human patient, wherein the treatment comprises administration of the pharmaceutical composition by intratumoral injection at a dose of 0.10-10.0 mg of the mRNA therapeutic agent in a dosing regimen comprising a first dosing cycle and at least one subsequent dosing cycle, wherein the first and subsequent dosing cycles comprise administration of the mRNA therapeutic at a dosing interval selected from once a week, once every 2 weeks, once every 3 weeks, and once every 4 weeks for a duration of time, wherein the dosing intervals for the first and subsequent dosing cycles are different, and wherein the LNP encapsulated mRNA therapeutic agent comprises three mRNA drug substances:

(i) a first mRNA encoding a human OX40L polypeptide comprising an ORF comprising a nucleotide sequence at least 80% identical to the nucleotide sequence set forth in SEQ ID NO: 4 or comprises the nucleotide sequence set forth in SEQ ID NO: 4;

(ii) a second mRNA encoding a human IL-23 polypeptide comprising an ORF comprising a nucleotide sequence at least 80% identical to the nucleotide sequence set forth in SEQ ID NO: 25 or comprises the nucleotide sequence set forth in SEQ ID NO: 25; and

(iii) a third mRNA encoding a human IL-36γ polypeptide comprising an ORF comprising a nucleotide sequence at least 80% identical to the nucleotide sequence set forth in SEQ ID NO: 28 or comprises the nucleotide sequence set forth in SEQ ID NO: 28.

In further aspects, the disclosure provides a kit comprising a container comprising a pharmaceutical composition comprising: an LNP encapsulated mRNA therapeutic agent; and a pharmaceutically acceptable carrier, and instructions for use in treating advanced or metastatic solid tumor malignancy in a human patient, wherein the treatment comprises administration of the pharmaceutical composition by intratumoral injection at a dose of 0.10-10.0 mg of the mRNA therapeutic agent, wherein the solid tumor malignancy is selected from the group consisting of: a bladder cancer, an immune checkpoint inhibitor (CPI)-refractory melanoma, a neoadjuvant melanoma, and a non-small cell lung carcinoma, and wherein the LNP encapsulated mRNA therapeutic agent comprises three mRNA drug substances:

(i) a first mRNA encoding a human OX40L polypeptide comprising an ORF comprising a nucleotide sequence at least 80% identical to the nucleotide sequence set forth in SEQ ID NO: 4 or comprises the nucleotide sequence set forth in SEQ ID NO: 4;

(ii) a second mRNA encoding a human IL-23 polypeptide comprising an ORF comprising a nucleotide sequence at least 80% identical to the nucleotide sequence set forth in SEQ ID NO: 25 or comprises the nucleotide sequence set forth in SEQ ID NO: 25; and

(iii) a third mRNA encoding a human IL-36γ polypeptide comprising an ORF comprising a nucleotide sequence at least 80% identical to the nucleotide sequence set forth in SEQ ID NO: 28 or comprises the nucleotide sequence set forth in SEQ ID NO: 28.

In yet further aspects, the disclosure provides a kit comprising a container comprising a pharmaceutical composition comprising: an LNP encapsulated mRNA therapeutic agent; and a pharmaceutically acceptable carrier, wherein the pharmaceutical composition comprises about 2 mg/mL of the mRNA therapeutic agent, and instructions for use in treating advanced or metastatic solid tumor malignancy or lymphoma in a human patient, wherein the treatment comprises administration of the pharmaceutical composition, optionally by by intratumoral injection, at a dose of 0.10-10.0 mg of the mRNA therapeutic agent in a dosing regimen from 7 to 28 days, and wherein the LNP encapsulated mRNA therapeutic agent comprises three mRNA drug substances:

(i) a first mRNA encoding a human OX40L polypeptide comprising an ORF comprising a nucleotide sequence at least 80% identical to the nucleotide sequence set forth in SEQ ID NO: 4 or comprises the nucleotide sequence set forth in SEQ ID NO: 4;

(ii) a second mRNA encoding a human IL-23 polypeptide comprising an ORF comprising a nucleotide sequence at least 80% identical to the nucleotide sequence set forth in SEQ ID NO: 25 or comprises the nucleotide sequence set forth in SEQ ID NO: 25; and

(iii) a third mRNA encoding a human IL-36γ polypeptide comprising an ORF comprising a nucleotide sequence at least 80% identical to the nucleotide sequence set forth in SEQ ID NO: 28 or comprises the nucleotide sequence set forth in SEQ ID NO: 28.

In some aspects, the disclosure provides a kit comprising a container comprising a pharmaceutical composition comprising: an LNP encapsulated mRNA therapeutic agent; and a pharmaceutically acceptable carrier, wherein the pharmaceutical composition comprises about 2 mg/mL of the mRNA therapeutic agent, and instructions for use in treating advanced or metastatic solid tumor malignancy or lymphoma in a human patient, wherein the treatment comprises administration of the pharmaceutical composition by intratumoral injection at a dose of 0.10-10.0 mg of the mRNA therapeutic agent in a dosing regimen comprising a first dosing cycle and at least one subsequent dosing cycle, wherein the first and subsequent dosing cycles comprise administration of the mRNA therapeutic at a dosing interval selected from once a week, once every 2 weeks, once every 3 weeks, and once every 4 weeks for a duration of time, wherein the dosing intervals for the first and subsequent dosing cycles are different, and wherein the LNP encapsulated mRNA therapeutic agent comprises three mRNA drug substances:

(i) a first mRNA encoding a human OX40L polypeptide comprising an ORF comprising a nucleotide sequence at least 80% identical to the nucleotide sequence set forth in SEQ ID NO: 4 or comprises the nucleotide sequence set forth in SEQ ID NO: 4;

(ii) a second mRNA encoding a human IL-23 polypeptide comprising an ORF comprising a nucleotide sequence at least 80% identical to the nucleotide sequence set forth in SEQ ID NO: 25 or comprises the nucleotide sequence set forth in SEQ ID NO: 25; and

(iii) a third mRNA encoding a human IL-36γ polypeptide comprising an ORF comprising a nucleotide sequence at least 80% identical to the nucleotide sequence set forth in SEQ ID NO: 28 or comprises the nucleotide sequence set forth in SEQ ID NO: 28.

In other aspects, the disclosure provides a kit comprising a container comprising a pharmaceutical composition comprising: an LNP encapsulated mRNA therapeutic agent; and a pharmaceutically acceptable carrier, wherein the pharmaceutical composition comprises about 2 mg/mL of the mRNA therapeutic agent, and instructions for use in treating advanced or metastatic solid tumor malignancy in a human patient, wherein the treatment comprises administration of the pharmaceutical composition by intratumoral injection at a dose of 0.10-10.0 mg of the mRNA therapeutic agent, wherein the solid tumor malignancy is selected from the group consisting of: a bladder cancer, an immune checkpoint inhibitor (CPI)-refractory melanoma, a neoadjuvant melanoma, and a non-small cell lung carcinoma, and wherein the LNP encapsulated mRNA therapeutic agent comprises three mRNA drug substances:

(i) a first mRNA encoding a human OX40L polypeptide comprising an ORF comprising a nucleotide sequence at least 80% identical to the nucleotide sequence set forth in SEQ ID NO: 4 or comprises the nucleotide sequence set forth in SEQ ID NO: 4;

(ii) a second mRNA encoding a human IL-23 polypeptide comprising an ORF comprising a nucleotide sequence at least 80% identical to the nucleotide sequence set forth in SEQ ID NO: 25 or comprises the nucleotide sequence set forth in SEQ ID NO: 25; and

(iii) a third mRNA encoding a human IL-36γ polypeptide comprising an ORF comprising a nucleotide sequence at least 80% identical to the nucleotide sequence set forth in SEQ ID NO: 28 or comprises the nucleotide sequence set forth in SEQ ID NO: 28.

In any of the foregoing or related aspects of the use or kits of the present disclosure, each of the first mRNA, second mRNA, and third mRNA comprise a 3′ untranslated region (UTR) comprising at least one microRNA-122 (miR-122) binding site. In some aspects, the miR-122 binding site is a miR-122-3p binding site or a miR-122-5p binding site. In some aspects, the miR-122-5p binding site comprises the nucleotide sequence set forth in SEQ ID NO: 20. In some aspects, the 3′UTR comprises a nucleotide sequence at least 90% identical to the sequence set forth in SEQ ID NO: 17 or comprises the nucleotide sequence as set forth in SEQ ID NO: 17. In some aspects, each of the first, second, and third mRNAs comprise a 5′cap, a 5′ untranslated region (UTR), and a poly-A tail of about 100 nucleotides in length. In some aspects, the 5′UTR comprises a nucleotide sequence at least 90% identical to the sequence set forth in SEQ ID NO: 15 or comprises the nucleotide sequence as set forth in SEQ ID NO: 15. In some aspects, the 5′UTR comprises a nucleotide sequence at least 90% identical to the sequence set forth in SEQ ID NO: 16 or comprises the nucleotide sequence as set forth in SEQ ID NO: 16.

In any of the foregoing or related aspects of the use or kits of the present disclosure, the LNP encapsulated mRNA therapeutic agent comprises three mRNA drug substances, wherein:

(i) the first mRNA encoding the human OX40L polypeptide comprises a nucleotide sequence at least 90% identical to the nucleotide sequence set forth in SEQ ID NO: 5 or comprises the nucleotide sequence set forth in SEQ ID NO: 5;

(ii) the second mRNA encoding a human IL-23 polypeptide comprises a nucleotide sequence at least 90% identical to the nucleotide sequence set forth in SEQ ID NO: 26 or comprises the nucleotide sequence set forth in SEQ ID NO: 26; and

(iii) the third mRNA encoding a human IL-36γ polypeptide comprises a nucleotide sequence at least 90% identical to the nucleotide sequence set forth in SEQ ID NO: 29 or comprises the nucleotide sequence set forth in SEQ ID NO: 29.

In any of the foregoing or related aspects of the use or kits of the present disclosure, the first, second, and third mRNAs are formulated in the lipid nanoparticle at a mass ratio of OX40L:IL-23:IL-36γ of 1:1:2.

In any of the foregoing or related aspects of the use or kits of the present disclosure, each of the first, second and third mRNAs is chemically modified.

In any of the foregoing or related aspects of the use or kits of the present disclosure, each of the first, second, and third mRNAs is fully modified with chemically-modified uridines. In some aspects, the chemically-modified uridines are N1-methylpseudouridines (m1ψ).

In any of the foregoing or related aspects of the use or kits of the present disclosure, each of the first, second and third mRNAs is fully modified with 5-methylcytosine or is fully modified with N1-methylpseudouridines (m1ψ) and 5-methylcytosine.

In any of the foregoing or related aspects of the use or kits of the present disclosure, the LNP comprises a compound having the formula:

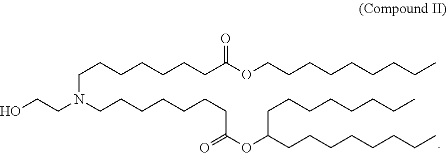 [SMILES: CCCCCCCCCOC(=O)CCCCCCCN(CCO)CCCCCCCC(=O)OC(CCCCCCCC)CCCCCCCC]

In some aspects, the LNP further comprising a phospholipid, a structural lipid, and a PEG lipid. In some aspects, the LNP comprises a molar ratio of about 20-60% ionizable amino lipid, about 5-25% phospholipid, about 25-55% structural lipid, and about 0.5-1.5% PEG lipid. In some aspects, the LNP comprises a molar ratio of about 50% ionizable amino lipid, about 10% phospholipid, about 38.5% structural lipid, and about 1.5% PEG lipid. In some aspects, the LNP comprises a molar ratio of about 50% ionizable amino lipid, about 10% phospholipid, about 38.5% cholesterol, and about 1.5% PEG-DMG. In some aspects, the LNP comprises a molar ratio of: (1) 40-60% ionizable amino lipid, 8-16% phospholipid, 30-45% sterol, and 1-5% PEG modified lipid; or (2) 45-65% ionizable amino lipid, 5-10% phospholipid, 25-40% sterol, and 0.5-5% PEG modified lipid. In some aspects, the LNP comprises a molar ratio of 40-60% ionizable amino lipid, 8-16% phospholipid, 30-45% sterol, and 1-5% PEG modified lipid. In other aspects, the LNP comprises a molar ratio of 45-65% ionizable amino lipid, 5-10% phospholipid, 25-40% sterol, and 0.5-5% PEG modified lipid. In some aspects, the LNP comprises a molar ratio of: (1) 40-60% Compound II, 8-16% DSPC, 30-45% cholesterol, and 1-5% PEG DMG; or (2) 45-65% Compound II, 5-10% DSPC, 25-40% cholesterol, and 0.5-5% PEG DMG. In some aspects, the LNP comprises a molar ratio of 40-60% Compound II, 8-16% DSPC, 30-45% cholesterol, and 1-5% PEG DMG. In some aspects, the LNP comprises a molar ratio of 45-65% Compound II, 5-10% DSPC, 25-40% cholesterol, and 0.5-5% PEG DMG.

### BRIEF DESCRIPTION OF FIGURES

FIGS. 1A-1E show the effect of doublet combination therapy and triplet combination therapy using mRNAs encoding IL-23, IL-36γ, and OX40L, wherein each mRNA comprises a miR-122 binding site. FIG. 1A shows doublet treatment with OX40L/IL-36γ encoding mRNAs. One complete response was observed. FIG. 1B shows doublet combination treatment with IL-23/IL-36γ encoding mRNAs. Four complete responses were observed. FIG. 1C shows doublet combination treatment with IL-23/OX40L encoding mRNAs. Seven complete responses were observed. FIG. 1D shows treatment with triplet mRNA combination encoding OX40L/IL-23/IL-36γ. Eleven complete responses were observed. FIG. 1E shows treatment with negative control mRNA (non-translating mRNA encoding for OX40L).

FIGS. 2A-2E show in vivo tumor efficacy in both primary treated and untreated distal tumors with Compound II-based LNPs comprising doublet mRNA therapy encoding IL-23/IL-36γ (FIG. 2A), OX40L/IL-23 (FIG. 2B) and triplet mRNAs encoding OX40L/IL-23/IL-36γ (FIG. 2C). FIG. 2A shows the effect of the doublet mRNA therapy (mRNA encoding IL-23 and mRNA encoding IL-36γ) on the combined tumor volume. FIG. 2B shows the effect of the doublet mRNA therapy (mRNA encoding IL-23 and mRNA encoding OX40L) on the combined tumor volume. FIG. 2C shows the effect of the triplet mRNA therapy (mRNA encoding OX40L/IL-23/IL-36γ) on the combined tumor volume. FIG. 2D shows the effect of the negative control mRNA (non-translating mRNA encoding for OX40L) on the combined tumor volume. FIG. 2E shows a schematic description of the MC38-S dual flank model used in the experiments.

FIG. 2F shows survival curves in the MC38 dual flank mice model in which mice were treated with control, doublet combinations or triplet mRNA therapy. Mice administered OX40L/LL-23/IL-36γ triplet mRNAs show 100% (n=20) survival rate. All mice that were not treated or given negative control mRNA were dead by approximately 40 days post-implantation of the MC38 tumor. Seventy percent of mice administered either OX40L/IL-23 or IL-23/IL-36γ doublet mRNAs survived at 100 days post-implantation with MC38.

FIGS. 3A-3D show in vivo anti-tumor efficacy of triplet mRNA therapy combined with an anti-PD-L1 antibody (10F.9G2) in immunosuppressive MC38 tumors. FIG. 3A shows tumor growth in animals treated with intraperitoneal injections of anti-PD-L1 antibody (10F.9G2) alone. FIG. 3B shows tumor growth in animals treated with intratumoral injections of triplet mRNA therapy. FIG. 3C shows tumor growth in animals treated with intratumoral injections of triplet mRNA therapy plus anti-PD-L1 antibody (10F.9G2). Vertical dashed lines indicate day of administration of the control antibody, the anti-PD-L1 antibody, the triplet mRNA therapy, or the triplet mRNA therapy plus anti-PD-L1 antibody. FIG. 3D shows tumor growth in animals treated with intratumoral injections negative control mRNA (non-translating mRNA encoding for OX40L).

FIGS. 4A-4D show in vivo anti-tumor efficacy of triplet mRNA therapy combined with an anti-CTLA-4 antibody (9D9) in immunosuppressive MC38 tumors. FIG. 4A shows tumor growth in animals treated with intraperitoneal injections of anti-CTLA-4 antibody (9D9) alone. FIG. 4B shows tumor growth in animals treated with intratumoral injections of triplet mRNA therapy. FIG. 4C shows tumor growth in animals treated with intratumoral injections of triplet mRNA therapy plus anti-CTLA-4 antibody (9D9). Vertical dashed lines indicate day of administration of the control antibody, the anti-CTLA-4 antibody, the triplet mRNA therapy, or the triplet mRNA therapy plus anti-CTLA-4 antibody. FIG. 4D shows tumor growth in animals treated with intratumoral injections of negative control mRNA (non-translating mRNA encoding for OX40L).

FIG. 5 provides a schematic depicting the clinical study design to evaluate mRNA-TRIPLET (mRNAs encoding OX40L/IL-23/IL-36γ) administered alone and in combination with checkpoint inhibitors: durvalumab (anti-PD-L1), or tremelimumab (anti-CTLA-4). The study comprises a dose escalation phase with three arms: mRNA-TRIPLET alone (Arm A), mRNA-TRIPLET with durvalumab (Arm B), and mRNA-TRIPLET with tremelimumab (Arm C). Once maximum tolerated dose (MTD) and/or recommended dose for expansion (RDE) is defined for mRNA-TRIPLET alone and in combination with checkpoint inhibitors, a dose expansion phase will be conducted to assess anti-tumor activity of mRNA-TRIPLET and durvalumab in Triple-negative breast cancer (TNBC), head and neck squamous cell carcinoma (HNSCC), non-Hodgkin lymphoma (NHL), and urothelial cancer (1 L or 2 L+); or the anti-tumor activity of mRNA-TRIPLET and tremelimumab in melanoma.

FIG. 6 provides a schematic showing the dosing schedule of Arms A and B described in FIG. 5 and indications of when biopsies, including optional biopsies, were obtained for analysis. C1D2-3=cycle 1, between days 2-3; C2D1=cycle 2, day 1 (biopsy taken less than 72 hours before the first dose of cycle 2); C1D15=cycle 1, day 15; C3D2-3=cycle 3, between days 2-3. CR=complete responder; PR=partial responder; PD=progressive disease.

FIG. 7 is a Swimmers plot showing tumor assessment over time per RECIST 1.1 in 17 patients on Arm A with duration on study up to 16 weeks, and 12 patients on Arm B up to 28 weeks on study and continuing at time of data cut off.

FIG. 8 is images from CT scans of a patient having squamous-cell bladder cancer and treated under Arm B with 0.5 mg dose of mRNA-TRIPLET. CT scans were taken at cycle 1 day 1 (C1D1) and cycle 3 day 1 (C3D1). The circles indicate tumor response. The top row at baselines shows a target lesion of 7.5 cm left rib body lesion with soft tissue component per RECIST 1.1, which had virtually resolved by C3D1. Bottom row shows a non-target bladder lesion that had virtually resolved by C3D1.

FIG. 9 is a Waterfall plot of maximum % change from baseline in sum of diameters of target lesions based on investigator assessment per RECIST 1.1. Change is shown for injected and uninjected tumors in patients from Arm A and Arm B. PD=progressive disease; SD=stable disease; PR=partial response.

FIG. 10 provides graphs showing IL-36γ levels in plasma (left) or tumor (right) over time in patients from Arms A and B. Due to variability in plasma IL-36γ levels across samples, fold changes from baseline were calculated (middle). Plasma was evaluated before treatment (screening) and 3 hours, 6 hours, 24 hours and 7 days post-treatment, whereas tumor levels were evaluated before treatment (screening) and at C1D2, C1D15, and C2D1. The legend provided applies to FIGS. 10-15 and 18.

FIG. 11 provides graphs showing IL-23 levels in plasma (left) or tumor (right) over time in patients from Arms A and B. Plasma was evaluated before treatment (screening) and 3 hours, 6 hours, 24 hours and 7 days post-treatment, whereas tumor levels were evaluated before treatment (screening) and at C1D2, C1D15, and C2D1.

FIG. 12 provides graphs showing IFN-γ levels in plasma (left) or tumor (right) over time in patients from Arms A and B. Plasma was evaluated before treatment (screening) and 3 hours, 6 hours, 24 hours and 7 days post-treatment, whereas tumor levels were evaluated before treatment (screening) and at C1D2, C1D15, and C2D1.

FIG. 13 provides graphs showing TNF-α levels in plasma (left) or tumor (right) over time in patients from Arms A and B. Plasma was evaluated before treatment (screening) and 3 hours, 6 hours, 24 hours and 7 days post-treatment, whereas tumor levels were evaluated before treatment (screening) and at C1D2, C1D15, and C2D1.

FIG. 14 provides a graph showing IL-22 levels in plasma over time in patients from Arms A and B. Plasma was evaluated before treatment (screening) and 3 hours, 6 hours, 24 hours and 7 days post-treatment.

FIG. 15 provides a graph showing IL-6 levels in plasma over time in patients from Arms A and B. Plasma was evaluated before treatment (screening) and 3 hours, 6 hours, 24 hours and 7 days post-treatment.

FIG. 16 provides a chart showing results of PD-L1 staining in tumor cells and immune cells pre- and post-treatment in patients from Arms A and B. BOR=best overall response; PD=progressive disease; SD=stable disease; PR=partial responder; NE=not evaluable; CRC=colorectal cancer; MCC=Merkel Cell carcinoma; TNBC=triple negative breast cancer; HNSCC=head and neck squamous cell carcinoma.

FIG. 17 provides representative images of PD-L1 staining in the tumor microenvironment from the patient having squamous-cell bladder cancer. Staining was done in biopsies taken pre-treatment and at C1D15 and C2D1. Dark staining shows PD-L1 expression.

FIG. 18 provides graphs showing % PD-L1+ tumor-associated immune cells from biopsies taken before treatment (pre) and at C1D2 (left), C1D15 (middle), and C2D1 (right).

FIG. 19 provides a schematic depicting the clinical study design to evaluate mRNA-TRIPLET (mRNAs encoding OX40L/IL-23/IL-36γ) administered alone and in combination with checkpoint inhibitor durvalumab (anti-PD-L1). The study comprises a dose escalation phase with two arms: mRNA-TRIPLET alone (Arm A), and mRNA-TRIPLET with durvalumab (Arm B), along with a dose exploration arm for cutaneous melanoma with mRNA-TRIPLET alone or in combination with durvalumab (Arm C). Once maximum tolerated dose (MTD) and/or recommended dose for expansion (RDE) is defined for mRNA-TRIPLET alone and in combination with checkpoint inhibitor, a dose expansion phase will be conducted to assess anti-tumor activity of mRNA-TRIPLET and durvalumab in Triple-negative breast cancer (TNBC), head and neck squamous cell carcinoma (HNSCC), non-Hodgkin lymphoma (NHL), urothelial cancer (1 L or 2 L+), checkpoint inhibitor (CPI)-refractory melanoma and CPI-refractory non-small cell lung carcinoma (NSCLC).

FIG. 20 provides a schematic showing the dosing schedule of Arms A, B and C described in FIG. 19 and indications of when biopsies, including optional biopsies, were obtained for analysis. C1D2-3=cycle 1, between days 2-3; C2D1=cycle 2, day 1 (biopsy taken less than 72 hours before the first dose of cycle 2); C1D15=cycle 1, day 15; C3D2-3=cycle 3, between days 2-3. PR=partial responder; CR=complete responder; PD=progressive disease.

### DETAILED DESCRIPTION

The present disclosure is directed to methods of treating solid tumor and/or lymphoma in a human patient by administering an effective amount of mRNAs encoding human OX40L, human IL-23 and human IL-36γ polypeptides. In some aspects, the mRNAs are encapsulated in a lipid nanoparticle. In some aspects, administering an effective amount of the mRNA combination encoding human OX40L, IL-23 and IL-36γ polypeptides reduces or decreases the size of a tumor (e.g., the tumor which has been injected and/or a proximal, un-injected tumor) in a triple negative breast cancer (TNBC), head and neck squamous cell carcinoma (HNSCC), non-Hodgkin lymphoma (NHL), bladder cancer (e.g., urothelial cancer), squamous-cell bladder cancer, bladder adenocarcinoma, or a melanoma cancer patient. In some aspects, administering an effective amount of the mRNA combination encoding human OX40L, IL-23 and IL-36γ polypeptides reduces or decreases the size of a tumor (e.g., the tumor which has been injected and/or a proximal, un-injected tumor) in a TNBC, HNSCC, NHL, bladder cancer (e.g., urothelial cancer), squamous-cell bladder cancer, bladder adenocarcinoma, non-small cell lung carcinoma (NSCLC), checkpoint inhibitor (CPI)-refractory NSCLC, melanoma, CPI-refractory melanoma, or neo-adjuvant melanoma cancer patient. In some aspects, administering an effective amount of mRNAs encoding human OX40L, IL-23 and IL-36γ polypeptides induces a specific cell-mediated immune response with systemic anti-tumor effects in a cancer patient having solid tumor malignancy or lymphoma. In some aspects, the expression of human OX40L, IL-23 and IL-36γ polypeptides in tumor cells and/or in immune cells in the tumor microenvironment is increased after administration of mRNAs encoding human OX40L, IL-23 and IL-36γ polypeptides.

### Methods of Use and Dosing Regimens

In some embodiments, the present disclosure provides methods of intratumoral (ITu) administration of an LNP encapsulated mRNAs therapeutic agent comprising three mRNA drug substances encoding human OX40L, IL-23 and IL-36γ polypeptides for treating solid tumor malignancies or lymphoma in a subject.

In some embodiments, the methods described herein comprise administering to the subject an LNP encapsulated mRNA therapeutic agent comprising three mRNA drug substances encoding human OX40L, IL-23 and IL-36γ of the disclosure, and pharmaceutical compositions suitable for ITu injection.

Compositions of the disclosure are administered to the subject in an effective amount. In general, an effective amount of the composition will allow for efficient production of the encoded polypeptide in cells of the subject. Metrics for efficiency may include polypeptide translation (indicated by polypeptide expression), level of mRNA degradation, and immune response indicators.

The methods of the disclosure for treating solid tumor malignancies or lymphomas are used in a variety of clinical or therapeutic applications. For example, the methods are used to stimulate anti-tumor immune responses in a subject with solid tumor malignancies or lymphomas. The mRNA and compositions of the present disclosure are useful in methods for treating or delaying progression of solid tumor malignancy or lymphoma in a subject, e.g., a human patient by intratumoral injection. The injection can be in a single injection at a single site or multiple injections at one or more sites (one or more tumors). The injection can be a bolus injection or a continuous infusion.

A suitable dose of an mRNA is a dose which treats or delays progression of solid tumor malignancy or lymphoma a human patient, and may be affected by a variety of factors including, e.g., the age, sex, and weight of a subject to be treated and the particular mRNA to be used. Other factors affecting the dose administered to the subject include, e.g., the type or severity of the malignancy or lymphoma in the patient. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject.

In some embodiments, a subject is administered at least one mRNA composition described herein. In related embodiments, the subject is provided with or administered a nanoparticle (e.g., a lipid nanoparticle) comprising the mRNA(s). In further related embodiments, the subject is provided with or administered a pharmaceutical composition of the disclosure to the subject. In particular embodiments, the pharmaceutical composition comprises an mRNA(s) as described herein, or it comprises a nanoparticle comprising the mRNA(s). In particular embodiments, the mRNA(s) is present in a nanoparticle, e.g., a lipid nanoparticle. In particular embodiments, the mRNA(s) or nanoparticle is present in a pharmaceutical composition, e.g., a composition suitable for intratumoral injection.

In some embodiments, the mRNA(s), nanoparticle, or pharmaceutical composition is administered to the patient parenterally, e.g., intratumorally. In particular embodiments, the subject is a mammal, e.g., a human. In various embodiments, the subject is provided with an effective amount of the mRNA(s).

Suitable doses for human patients can be evaluated in, e.g., a Phase I dose escalation study. Data obtained from the cell culture assays and animal studies can be used in formulating a range of dosage for use in humans. The dosage of such mRNA described herein lies generally within a range of local concentrations of the mRNA that include the ED50 with little or no toxicity. The dosage may vary within this range depending upon the dosage form employed and the route of administration utilized. For the mRNA and compositions described herein, the therapeutically effective dose can be estimated initially from cell culture assays. A dose can be formulated in animal models to achieve a therapeutically effective concentration within the local site that includes the IC50 (i.e., the concentration of the mRNA which achieves a half-maximal inhibition of symptoms) as determined in cell culture. Such information can be used to more accurately determine useful doses in humans.

In some embodiments, the frequency of dosing will take into account the pharmacokinetic parameters of the mRNA in the formulation used. In certain embodiments, a clinician will administer the composition until a dosage is reached that achieves or maintains the desired effect. In some embodiments, the desired effect is tumor size reduction or resolution of the injected or uninjected tumors. In some embodiments, the desired effect is expression of OX40L, IL-23 and IL-36γ within the tumor. In some embodiments, achievement of a desired effect occurs immediately after administration of a dose. In some embodiments, achievement occurs at any point in time following administration. In some embodiments, achievement occurs at any point in time during a dosing interval. In some embodiments, achievement of a desired effect is determined by analyzing a biological sample (e.g., tumor biopsy) immediately after administration of a dose, at any point in time following administration of a dose, at any point in time during a doing interval, or combinations thereof.

In some embodiments, maintenance of a desired effect (e.g., OX40L, IL-23 or IL-36γ protein expression) is determined by analyzing a biological sample (e.g., tumor biopsy) at least once during a dosing interval. In some embodiments, maintenance of a desired effect (e.g., OX40L, IL-23 or IL-36γ protein expression) is determined by analyzing a biological sample (e.g., tumor biopsy) at regular intervals during a dosing interval. In some embodiments, maintenance of a desired effect (e.g., OX40L, IL-23 or IL-36γ protein expression) is determined by analyzing a biological sample (e.g., tumor biopsy) before a subsequent dose is administered.

### Tumor Size Reduction or Growth Inhibition

In some embodiments, the subject has a solid tumor malignancy or lymphoma as described supra. In some embodiments, the human OX40L, IL-23 and IL-36γ encoding mRNAs are administered locally to a tumor (i.e., intratumorally). In some embodiments, administration of the human OX40L, IL-23 and IL-36γ encoding mRNAs to a tumor reduces the size or volume, or inhibits the growth of the injected tumor. In some embodiments, administration of the human OX40L, IL-23 and IL-36γ encoding mRNAs to a tumor reduces the size or inhibits the growth of the injected tumor and an uninjected tumor in the subject. In some embodiments, the uninjected tumor is located near or proximal to the injected tumor. In some embodiments, the uninjected tumor is located distal to the injected tumor. In some embodiments, the reduction in size or inhibition of growth in an uninjected tumor is through an abscopal effect.

In some embodiments, reduction in tumor size is by at least 25%. In some embodiments, reduction in tumor size is by at least 50%. In some embodiments, reduction in tumor size is by at least 75%. In some embodiments, the tumor is completely resolved.

In some embodiments, a reduction in tumor size is measured by comparison to the size of patient's tumor at baseline, against an expected tumor size, against an expected tumor size based on a large patient population, or against the tumor size of a control population.

In some embodiments, tumor size is determined by visual methods, such as image scanning. Methods for determining tumor size and tumor volume are known to those of skill in the art.

### OX40L, IL-23 and IL-36γ Protein Expression

In some embodiments, human OX40L, IL-23 and IL-36γ protein expression are enhanced in a tumor administered OX40L, IL-23 and IL-36γ encoding mRNAs. In some embodiments, enhancement of human OX40L, IL-23 and IL-36γ protein expression are relative to expression prior to administration of the OX40L, IL-23 and IL-36γ encoding mRNAs. In some embodiments, a biopsy is obtained from the tumor before and after administration of the OX40L, IL-23 and IL-36γ encoding mRNAs, and protein expression are assessed.

In some embodiments, human OX40L, IL-23 and IL-36γ protein expression are increased by at least 1-fold, 2-fold, 3-fold, 4-fold, 5-fold, 6-fold, 7-fold, 8-fold, 9-fold, or 10-fold, on tumor cells or immune cells within a tumor at any point in time following administration of the OX40L, IL-23 and IL-36γ encoding mRNAs or composition of the disclosure, as determined by a method described herein. In some embodiments, OX40L, IL-23 and IL-36γ protein expression are increased by at least 1-fold, 2-fold, 3-fold, 4-fold, 5-fold, 6-fold, 7-fold, 8-fold, 9-fold, or 10-fold, on tumor cells or immune cells within a tumor at any point in time during the duration of a dosing interval, as determined by a method described herein. In some embodiments, OX40L, IL-23 and IL-36γ protein expression are increased by at least 1-fold, 2-fold, 3-fold, 4-fold, 5-fold, 6-fold, 7-fold, 8-fold, 9-fold, or 10-fold, on tumor cells or immune cells within a tumor at any point in time during the duration of a dosing interval comprising a duration of 7-35 days, as determined by a method described herein. In some embodiments, OX40L, IL-23 and IL-36γ protein expression are increased by at least 1-fold, 2-fold, 3-fold, 4-fold, 5-fold, 6-fold, 7-fold, 8-fold, 9-fold, or 10-fold, on tumor cells or immune cells within a tumor at any point in time during the duration of a dosing interval comprising a duration of 14-28 days, as determined by a method described herein. In some embodiments, OX40L, IL-23 and IL-36γ protein expression are increased by at least 1-fold, 2-fold, 3-fold, 4-fold, 5-fold, 6-fold, 7-fold, 8-fold, 9-fold, or 10-fold, on tumor cells or immune cells within a tumor at any point in time during the duration of a dosing interval comprising a duration of 21-28 days, as determined by a method described herein. In some embodiments, OX40L, IL-23 and IL-36γ protein expression are increased by at least 1-fold, 2-fold, 3-fold, 4-fold, 5-fold, 6-fold, 7-fold, 8-fold, 9-fold, or 10-fold, on tumor cells or immune cells within a tumor on, during or after day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or day 35 of the dosing interval, as determined by a method described herein.

Methods for determining human OX40L, IL-23 and IL-36γ protein expression on appropriate cell types, such as immune cells or tumor cells located within a tumor are known to those of skill in the art and described herein. Such methods include, but are not limited to, quantitative immunofluorescence (QIF), flow cytometry, reverse transcription polymerase chain reaction (RT-PCR), competitive RT-PCR, real-time RT-PCR, RNase protection assay (RPA), northern blotting, nucleic acid microarray using DNA, western blotting, enzyme-linked immunosorbent assay (ELISA), radioimmunoassay (RIA), tissue immunostaining, immunoprecipitation assay, complement fixation assay, fluorescence-activated cell sorting (FACS), mass spectrometry, magnetic bead-antibody immunoprecipitation, or protein chip.

### Enhancing Anti-Tumor Immune Responses

In some embodiments, the disclosure provides a method for enhancing an immune response in a subject with solid tumor malignancy or lymphoma. In some embodiments, the disclosure provides a method for enhancing an immune response to a solid tumor. In some embodiments, enhancing an immune response comprises stimulating cytokine production. In another embodiment, enhancing an immune response comprises enhancing cellular immunity (T cell responses), such as activating T cells. In some embodiments, enhancing an immune response comprises activating NK cells. Enhancement of an immune response in a subject can be evaluated by a variety of methods established in the art for assessing immune response, including but not limited to determining the level of T cell activation and NK cell activation by intracellular staining of activation markers in the area of the tumor.

In some embodiments, local administration of mRNAs encoding a human OX40L, IL-23 and IL-36γ polypeptides to a tumor induces T cell activation within the tumor. In some embodiments, the activation of T cells results in an anti-tumor immune response in the subject. In some embodiments, the activated T cells in the subject reduce or decrease the size of a tumor or inhibit the growth of a tumor in the subject. Activation of T cells can be measured using applications in the art such as measuring T cell proliferation; measuring cytokine production with enzyme-linked immunosorbant assays (ELISA) or enzyme-linked immunospot assays (ELISPOT); or detection of cell-surface markers associated with T cell activation (e.g., CD69, CD40L, CD137, CD25, CD71, CD26, CD27, CD28, CD30, CD154, and CD134) with techniques such as flow cytometry.

In some embodiments, the activated T cells are CD4⁺ cells, CD8⁺ cells, CD62⁺ (L-selectin⁺) cells, CD69⁺ cells, CD40L⁺ cells, CD137⁺ cells, CD25⁺ cells, CD71⁺ cells, CD26⁺ cells, CD27⁺ cells, CD28⁺ cells, CD30⁺ cells, CD45⁺ cells, CD45RA⁺ cells, CD45RO⁺ cells, CD11b⁺ cells, CD154⁺ cells, CD134⁺ cells, CXCR3⁺ cells, CCR4⁺ cells, CCR6⁺ cells, CCR7⁺ cells, CXCR5⁺ cells, Crth2⁺ cells, gamma delta T cells, or any combination thereof. In some embodiments, the activated T cells are Th₁ cells. In other embodiments, the T cells are Th₂ cells. In other embodiments, the activated T cells activated are cytotoxic T cells.

In some embodiments, local administration of mRNAs encoding human OX40L, IL-23 and IL-36γ polypeptides to a tumor induces T cell proliferation within the tumor. In some embodiments, T cell proliferation results in an anti-tumor immune response in the subject. In some embodiments, T cell proliferation in the subject reduce or decrease the size of a tumor or inhibit the growth of a tumor in the subject. T cell proliferation can be measured using applications in the art such as cell counting, viability staining, optical density assays, or detection of cell-surface markers associated with T cell activation (e.g., CD69, CD40L, CD137, CD25, CD71, CD26, CD27, CD28, CD30, CD154, and CD134) with techniques such as flow cytometry.

In some embodiments, local administration of mRNAs encoding human OX40L, IL-23 and IL-36γ polypeptides to a tumor induces infiltration of T cells to the tumor. In some embodiments, T cell infiltration results in an anti-tumor immune response in the subject. In some embodiments, T cell infiltration in the subject reduce or decrease the size of a tumor or inhibit the growth of a tumor in the subject. T cell infiltration in a tumor can be measured using applications in the art such as tissue sectioning and staining for cell markers, measuring local cytokine production at the tumor site, or detection of T cell-surface markers with techniques such as flow cytometry.

In some embodiments, the infiltrating T cells are CD4⁺ cells, CD8⁺ cells, CD62⁺ (L-selectin⁺) cells, CD69⁺ cells, CD40L⁺ cells, CD137⁺ cells, CD25⁺ cells, CD71⁺ cells, CD26⁺ cells, CD27⁺ cells, CD28⁺ cells, CD30⁺ cells, CD45⁺ cells, CD45RA⁺ cells, CD45RO⁺ cells, CD11b⁺ cells, CD154⁺ cells, CD134⁺ cells, CXCR3⁺ cells, CCR4⁺ cells, CCR6⁺ cells, CCR7⁺ cells, CXCR5⁺ cells, Crth2⁺ cells, gamma delta T cells, or any combination thereof. In some embodiments, the infiltrating T cells are Th₁ cells. In other embodiments, the infiltrating T cells are Th₂ cells. In other embodiments, the infiltrating T cells are cytotoxic T cells.

In some embodiments, local administration of mRNAs encoding human OX40L, IL-23 and IL-36γ polypeptides to a tumor increases the number of Natural Killer (NK) cells within the tumor. In some embodiments, the increase in the number of NK cells results in an anti-tumor immune response in the subject. In some embodiments, the increase in the number of NK cells reduces or decreases the size of a tumor or inhibits the growth of a tumor in the subject. Increases in the number of NK cells in a subject can be measured using applications in the art such as detection of NK cell-surface markers (e.g., CD335/NKp46; CD336/NKp44; CD337/NPp30) or intracellular NK cell markers (e.g., perforin; granzymes; granulysin).

In some embodiments, administration of mRNAs encoding OX40L, IL-23 and IL-36γ polypeptides increases the total number of NK cells in the tumor compared to the number of NK cells in a tumor that is not administered mRNAs encoding an OX40L, IL-23 and IL-36γ polypeptides.

In certain embodiments, the number of NK cells is increased at least about two-fold, at least about three-fold, at least about four-fold, at least about five-fold, at least about six-fold, at least about seven-fold, at least about eight-fold, at least about nine-fold, or at least about ten-fold compared to a control (e.g., saline or an mRNA without OX40L, IL-23 and IL-36γ expression).

In some embodiments, administration of mRNAs encoding OX40L, IL-23 and IL-36γ polypeptides increases cytokine levels. In some embodiments, the increased cytokines include IL-22, IL-6, TNFα, IFNγ, IL-8, IL-2, IL-10, IL-27 and MIP3α. In some embodiments, the increased cytokine levels are below the levels indicated as being associated with cytokine release syndrome (CRS). CRS is an acute systemic inflammatory syndrome characterized by fever and multiple organ dysfunction that can be triggered by a variety of infections and certain drugs, such as antibody-based therapies. IL-6, IL-10 and IFNγ are among the core cytokines that are consistently found to be elevated in serum of patients with CRS. In some embodiments, increased cytokine levels are at least 1-fold, 2-fold, 3-fold, 4-fold, or 5-fold less than the levels reported for CRS. In some embodiments, the mRNA therapeutic agent does not induce CRS.

In some embodiments, administration of mRNAs encoding OX40L, IL-23 and IL-36γ polypeptides increases expression of PD-L1 in immune cells in the tumor microenvironment. In some embodiments, administration of mRNAs encoding OX40L, IL-23 and IL-36γ polypeptides increases expression of PD-L1 in tumor cells. In some embodiments, PD-L1 expression is increased from negative expression to low expression. In some embodiments, low expression is characterized as 25% or less PD-L1+ cells. In some embodiments, PD-L1 expression is increased from negative expression to high expression. In some embodiments, high expression is characterized as more than 25% PD-L1+ cells. In some embodiments, PD-L1 expression is increased from low expression to high expression.

In some embodiments, expression of a protein (e.g., IL-23, IL-36γ, PD-L1 etc.) is determined by measuring the amount of protein in a sample (e.g., plasma or tumor). Methods for measuring protein levels are known to those of skill in the art and described herein.

### Dosing

In some embodiments, the mRNA or composition can therefore be administered as a single dose or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In some embodiments, the dosing regimen is determined by the pharmacodynamics effects of the human OX40L, IL-23 and IL-36γ polypeptides. In some embodiments, the pharmacodynamics effects include an increase in T cells within tumors after administration. In some embodiments, the increase in T cells is maintained over a specified period of time (e.g., 14 days).

In some embodiments, the composition comprising mRNAs encoding human OX40L, IL-23 and IL-36γ is administered at a dosing interval comprising a duration of about 14-28 days or about 21-28 days. In some embodiments, the composition comprising mRNAs encoding human OX40L, IL-23 and IL-36γ is administered at a dosing interval comprising a duration of about 7-42 days, about 7-21 days, about 14-28 days, about 21-28 days, about 21-35 days, about 28-35 days, about 21-42 days, or about 28-42 days. In some embodiments, the dosing interval is about 7 days. In some embodiments, the dosing interval is about 14 days. In some embodiments, the dosing interval is about 21 days. In some embodiments, the dosing interval is about 28 days. In some embodiments, the dosing interval is about 35 days. In some embodiments, the dosing interval is about 42 days. In some embodiments, the dosing interval is at least about 7 days. In some embodiments, the dosing interval is at least about 14 days. In some embodiments, the dosing interval is at least about 21 days. In some embodiments, the dosing interval is at least about 28 days. In some embodiments, the dosing interval is at least about 35 days. In some embodiments, the dosing interval is at least about 42 days.

In some embodiments, the composition comprising mRNAs encoding human OX40L, IL-23 and IL-36γ is administered to a subject about every 7-42 days for a specified time period. In some embodiments, the composition comprising mRNAs encoding human OX40L, IL-23 and IL-36γ is administered to a subject about every 7-21 days for a specified time period. In some embodiments, the composition comprising mRNAs encoding human OX40L, IL-23 and IL-36γ is administered to a subject about every 14-21 days for a specified time period. In some embodiments, the composition comprising mRNAs encoding human OX40L, IL-23 and IL-36γ is administered to a subject about every 14-28 days for a specified time period. In some embodiments, the composition comprising mRNAs encoding human OX40L, IL-23 and IL-36γ is administered to a subject about every 21-28 days for a specified time period. In some embodiments, the composition comprising mRNAs encoding human OX40L, IL-23 and IL-36γ is administered to a subject about every 21-35 days for a specified time period. In some embodiments, the composition comprising mRNAs encoding human OX40L, IL-23 and IL-36γ is administered to a subject about every 28-42 days for a specified time period.

In some embodiments, the composition comprising mRNA encoding human OX40L, IL-23 and IL-36γ is administered to a subject for a first dosing cycle and at least one subsequent dosing cycle, wherein the first and subsequent dosing cycles each independently comprise administering a dose of the composition at a dosing interval of once a week, once every 2 weeks, once every 3 weeks, or once every 4 weeks, for a duration of time. In some embodiments, the first and subsequent dosing cycles comprise administering a dose of the composition at different dosing intervals (e.g., first dosing cycle comprises administering a dose of the composition at a dosing interval of once every 2 weeks, and at least one subsequent dosing cycle comprises administering a dose of the composition at a dosing interval of once every 4 weeks). In some embodiments, the subject is administered the composition in two or more subsequent dosing cycles after the first dosing cycle, wherein the two or more subsequent dosing cycles comprise the same or different dosing intervals.

In some embodiments, the composition comprising mRNAs encoding human OX40L, IL-23 and IL-36γ is administered to a subject about every 2 weeks for a first dosing cycle, and about every 4 weeks for at least one subsequent dosing cycle. In some embodiments, the composition comprising mRNAs encoding human OX40L, IL-23 and IL-36γ is administered to a subject about every 2 weeks for a first dosing cycle, and about every 4 weeks for at least three subsequent dosing cycles.

In some embodiments, the composition comprising mRNAs encoding human OX40L, IL-23 and IL-36γ is administered to a subject about every 7-14 days for a first dosing cycle, and about every 21-28 days for at least one second or subsequent dosing cycle. In some embodiments, the composition comprising mRNAs encoding human OX40L, IL-23 and IL-36γ is administered to a subject about every 7-21 days in a first dosing cycle, and about every 28-42 days for at least one second or subsequent dosing cycle.

In some embodiments, the composition comprising mRNAs encoding human OX40L, IL-23 and IL-36γ is administered to a subject for a first dosing cycle comprising administering a dose of the composition at a dosing interval of once every week for a duration of time, and at least one subsequent dosing cycle comprising administering a dose of the composition at a dosing interval of once a week, once every 2 weeks, once every 3 weeks, or once every 4 weeks for a duration of time. In some embodiments, the composition comprising mRNAs encoding human OX40L, IL-23 and IL-36γ is administered to a subject for a first dosing cycle comprising administering a dose of the composition at a dosing interval of once every 2 weeks for a duration of time, and at least one subsequent dosing cycle comprising administering a dose of the composition at a dosing interval of once every week, once every 2 weeks, once every 3 weeks or once every 4 weeks, for a duration of time. In some embodiments, the composition comprising mRNAs encoding human OX40L, IL-23 and IL-36γ is administered to a subject for a first dosing cycle comprising administering a dose of the composition at a dosing interval of once every 3 weeks for a duration of time, and at least one subsequent dosing cycle comprising administering a dose of the composition at a dosing interval of once a week, once every 2 weeks, once every 3 weeks or once every 4 weeks, for a duration of time. In some embodiments, the composition comprising mRNAs encoding human OX40L, IL-23 and IL-36γ is administered to a subject for a first dosing cycle comprising administering a dose of the composition at a dosing interval of once every 4 weeks for a duration of time, and at least one subsequent dosing cycle comprising administering a dose of the composition at a dosing interval of once a week, once every 2 weeks, once every 3 weeks or once every 4 weeks, for a duration of time.

In some embodiments, the composition comprising mRNAs encoding human OX40L, IL-23 and IL-36γ is administered to a subject for a first dosing cycle comprising administering a dose of the composition at a dosing interval of once every week for a duration of time, and at least one subsequent dosing cycle comprising administering a dose of the composition at a dosing interval of once every 4 weeks for a duration of time. In some embodiments, the composition comprising mRNAs encoding human OX40L, IL-23 and IL-36γ is administered to a subject for a first dosing cycle comprising administering a dose of the composition at a dosing interval of once every 2 weeks for a duration of time, and at least one subsequent dosing cycle comprising administering a dose of the composition at a dosing interval of once every 4 weeks for a duration of time. In some embodiments, the composition comprising mRNAs encoding human OX40L, IL-23 and IL-36γ is administered to a subject for a first dosing cycle comprising administering a dose of the composition at a dosing interval of once every 3 weeks for a duration of time, and at least one subsequent dosing cycle comprising administering a dose of the composition at a dosing interval of once every 4 weeks for a duration of time.

In some embodiments, the composition comprising mRNAs encoding human OX40L, IL-23 and IL-36γ is administered to a subject for a first dosing cycle comprising administering a dose of the composition at a dosing interval of once every week for a duration of time, and up to six subsequent dosing cycles each comprising administering a dose of the composition at a dosing interval of once every 4 weeks for a duration of time. In some embodiments, the composition comprising mRNAs encoding human OX40L, IL-23 and IL-36γ is administered to a subject for a first dosing cycle comprising administering a dose of the composition at a dosing interval of once every 2 weeks for a duration of time, and up to six subsequent dosing cycles each comprising administering a dose of the composition at a dosing interval of once every 4 weeks for a duration of time. In some embodiments, the composition comprising mRNAs encoding human OX40L, IL-23 and IL-36γ is administered to a subject for a first dosing cycle comprising administering a dose of the composition at a dosing interval of once every 3 weeks for a duration of time, and up to six subsequent dosing cycles each comprising administering a dose of the composition at a dosing interval of once every 4 weeks for a duration of time.

In some embodiments, a dosing cycle comprises at least one dose. In some embodiments, a dosing cycle comprises at least two doses. In some embodiments, a dosing cycle comprises at least three doses. In some embodiments, a dosing cycle comprises at least four doses. In some embodiments, a dosing cycle is for a duration of time. In some embodiments, a dosing cycle is about 7-42 days, about 7-21 days, about 14-28 days, about 21-28 days, about 21-35 days, about 28-35 days, about 21-42 days, or about 28-42 days. In some embodiments, a dosing cycle is 28 days. In some embodiments, a dosing cycle is 35 days. In some embodiments, a dosing cycle is 42 days. In some embodiments, a dosing cycle is 4 weeks.

In some embodiments, the composition is administered to a subject about every 7 days for a specified time period. In some embodiments, the composition is administered to a subject about every 14 days for a specified time period. In some embodiments, the composition is administered to a subject about every 21 days for a specified time period. In some embodiments, the composition is administered to a subject about every 28 days for a specified time period. In some embodiments, the composition is administered to a subject about every 35 days for a specified time period. In some embodiments, the composition is administered to a subject about every 42 days for a specified time period.

In some embodiments, the specified time period is determined by a clinician. In some embodiments, dosing occurs until a positive therapeutic outcome is achieved. For example, in some embodiments, dosing occurs until growth of cancer cells, tumor cells or tumors is inhibited. In some embodiments, dosing occurs until growth of cancer cells, tumor cells or tumors is reduced. In some embodiments, dosing occurs until there is no detection of cancer cells, tumor cells or tumors in a biological sample. In some embodiments, dosing occurs until progression of a cancer is delayed. In some embodiments, dosing occurs until progression of a cancer is inhibited. In some embodiments, the specified time period is determined once a positive therapeutic outcome is achieved.

In some embodiments, dosing of a composition comprising mRNAs encoding human OX40L, IL-23 and IL-36γ will occur indefinitely, or until a positive therapeutic outcome is achieved. In some embodiments, the dosing interval remains consistent. In some embodiments, the dosing interval changes as needed based on a clinician's assessment. In some embodiments, dosing occurs indefinitely to maintain remission of a cancer.

In some embodiments, the composition comprising mRNAs encoding human OX40L, IL-23 and IL-36γ is administered to a subject about every 7-42 days indefinitely, or until a positive therapeutic outcome is achieved. In some embodiments, the composition comprising mRNAs encoding human OX40L, IL-23 and IL-36γ is administered to a subject about every 7-21 days indefinitely, or until a positive therapeutic outcome is achieved. In some embodiments, the composition comprising mRNAs encoding human OX40L, IL-23 and IL-36γ is administered to a subject about every 14-21 days indefinitely, or until a positive therapeutic outcome is achieved. In some embodiments, the composition comprising mRNAs encoding human OX40L, IL-23 and IL-36γ is administered to a subject about every 14-28 days indefinitely, or until a positive therapeutic outcome is achieved. In some embodiments, the composition comprising mRNAs encoding human OX40L, IL-23 and IL-36γ is administered to a subject about every 21-28 days indefinitely, or until a positive therapeutic outcome is achieved. In some embodiments, the composition comprising mRNAs encoding human OX40L, IL-23 and IL-36γ is administered to a subject about every 21-35 days indefinitely, or until a positive therapeutic outcome is achieved. In some embodiments, the composition comprising mRNAs encoding human OX40L, IL-23 and IL-36γ is administered to a subject about every 28-42 days indefinitely, or until a positive therapeutic outcome is achieved.

In some embodiments, the composition is administered to a subject about every 7 days indefinitely, or until a positive therapeutic outcome is achieved. In some embodiments, the composition is administered to a subject about every 14 days indefinitely, or until a positive therapeutic outcome is achieved. In some embodiments, the composition is administered to a subject about every 21 days indefinitely, or until a positive therapeutic outcome is achieved. In some embodiments, the composition is administered to a subject about every 28 days indefinitely, or until a positive therapeutic outcome is achieved. In some embodiments, the composition is administered to a subject about every 35 days indefinitely, or until a positive therapeutic outcome is achieved. In some embodiments, the composition is administered to a subject about every 42 days indefinitely, or until a positive therapeutic outcome is achieved. In some embodiments, a dose of the composition is administered to a subject for at least two dosing cycles, or until a positive therapeutic outcome is achieved.

In certain embodiments, compositions of the disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.1 mg/kg to about 10 mg/kg, from about 1 mg/kg to about 5 mg/kg, from about 2 mg/kg to about 5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, or from about 0.1 mg/kg to about 1 mg/kg in a given dose, where a dose of 1 mg/kg provides 1 mg of mRNA or nanoparticle per 1 kg of subject body weight. In particular embodiments, a dose of about 0.005 mg/kg to about 5 mg/kg of mRNA or nanoparticle of the disclosure may be administrated.

In some embodiments, an mRNA composition is administered at a dose between about 0.010 mg/kg to about 0.5 mg/kg for a human patient. In some embodiments, the mRNA composition is administered at a dose between about 0.015 mg/kg to about 0.4 mg/kg. In some embodiments, the mRNA composition is administered at a dose between about 0.020 mg/kg to about 0.3 mg/kg. In some embodiments, the mRNA composition is administered at a dose between about 0.025 mg/kg to about 0.2 mg/kg. In some embodiments, the mRNA composition is administered at a dose between about 0.030 mg/kg to about 0.1 mg/kg.

In some embodiments, a mRNA composition is administered at a dose between about 0.5 mg to about 10.0 mg of mRNA for a human patient. In some embodiments, a composition is administered at a dose of 0.5 mg. In some embodiments, a composition is administered at a dose of 1.0 mg. In some embodiments, a composition is administered at a dose of 2.0 mg. In some embodiments, a composition is administered at a dose of 4.0 mg. In some embodiments, a composition is administered at a dose of 8.0 mg.

In some embodiments, a single dose may be administered, for example, prior to or after, or in lieu of a surgical procedure or in the instance of an acute disease, disorder, or condition. The specific therapeutically effective, prophylactically effective, or otherwise appropriate dose level for any particular patient will depend upon a variety of factors including the severity and identify of a disorder being treated, if any; the one or more mRNAs employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

### Combination Therapy

In some embodiments, a pharmaceutical composition of the disclosure may be administered in combination with another agent, for example, another therapeutic agent, a prophylactic agent, and/or a diagnostic agent. By “in combination with,” it is not intended to imply that the agents must be administered at the same time and/or formulated for delivery together, although these methods of delivery are within the scope of the present disclosure. For example, one or more compositions including one or more different mRNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the disclosure, or imaging, diagnostic, or prophylactic compositions thereof in combination with agents that improve their bioavailability, reduce and/or modify their metabolism, inhibit their excretion, and/or modify their distribution within the body.

Exemplary therapeutic agents that may be administered in combination with the compositions of the disclosure include, but are not limited to, cytotoxic, chemotherapeutic, hypomethylating agents, pro-apoptotic agents, small molecules/kinase inhibitors, immunostimulatory agents and other therapeutic agents including therapeutics approved for solid tumor malignancy or lymphoma, now or at a later date. Cytotoxic agents may include, for example, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, rachelmycin, and analogs thereof. Radioactive ions may also be used as therapeutic agents and may include, for example, radioactive iodine, strontium, phosphorous, palladium, cesium, iridium, cobalt, yttrium, samarium, and praseodymium. Other therapeutic agents may include, for example, antimetabolites (e.g., methotrexate, 6-mercaptopurine, 6-thioguanine, cytarabine, and 5-fluorouracil, and decarbazine), alkylating agents (e.g., mechlorethamine, thiotepa, chlorambucil, rachelmycin, melphalan, carmustine, lomustine, cyclophosphamide, busulfan, dibromomannitol, streptozotocin, mitomycin C, and cis-dichlorodiamine platinum (II) (DDP), and cisplatin), anthracyclines (e.g., daunorubicin and doxorubicin), antibiotics (e.g., dactinomycin, bleomycin, mithramycin, and anthramycin), and anti-mitotic agents (e.g., vincristine, vinblastine, taxol, and maytansinoids).

In some embodiments, the human OX40L, IL-23 and IL-36γ encoding mRNAs are administered to a subject having a solid tumor malignancy or lymphoma, wherein the subject has received or is receiving treatment with one or more anti-cancer agents. In some embodiments, the human OX40L, IL-23 and IL-36γ encoding mRNAs are administered in combination with one or more anti-cancer agents to a subject having a solid tumor malignancy or lymphoma. In some embodiments, the OX40L, IL-23 and IL-36γ encoding mRNAs and one or more anti-cancer agents are administered simultaneously or sequentially. In some embodiments, the OX40L, IL-23 and IL-36γ encoding mRNAs are administered after administration of one or more anti-cancer agents. In some embodiments, the OX40L, IL-23 and IL-36γ encoding mRNAs are administered before administration of one or more anti-cancer agents.

In some embodiments, the one or more anti-cancer agents are approved by the United States Food and Drug Administration. In other embodiments, the one or more anti-cancer agents are pre-approved by the United States Food and Drug Administration.

In some embodiments, the subject for the present methods has been treated with one or more standard of care therapies. In other embodiments, the subject for the present methods has not been responsive to one or more standard of care therapies or anti-cancer therapies.

In some embodiments, the subject has been previously treated with a PD-1 antagonist prior to a treatment with OX40L, IL-23 and IL-36γ encoding mRNAs. In some embodiments, the subject is treated with a monoclonal antibody that binds to PD-1 simultaneously with or subsequent to treatment with OX40L, IL-23 and IL-36γ encoding mRNAs. In some embodiments, the subject has been treated with an anti-PD-1 monoclonal antibody therapy prior to treatment with OX40L, IL-23 and IL-36γ encoding mRNAs. In some embodiments, the anti-PD-1 monoclonal antibody therapy comprises Nivolumab, Pembrolizumab, Pidilizumab, or any combination thereof.

In some embodiments, the anti-PD-1 antibody (or an antigen-binding portion thereof) useful for the disclosure is pembrolizumab. Pembrolizumab (also known as “KEYTRUDA®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. No. 8,900,587. Pembrolizumab has been approved by the FDA for the treatment of relapsed or refractory melanoma and advanced NSCLC.

In some embodiments, the anti-PD-1 antibody useful for the disclosure is nivolumab. Nivolumab (also known as “OPDIVO®”; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56). Nivolumab has shown activity in a variety of advanced solid tumors including renal cell carcinoma (renal adenocarcinoma, or hypernephroma), melanoma, and non-small cell lung cancer (NSCLC) (Topalian et al., 2012a; Topalian et al., 2014; Drake et al., 2013; WO 2013/173223).

In other embodiments, the anti-PD-1 antibody is MEDI0680 (formerly AMP-514), which is a monoclonal antibody against the PD-1 receptor. MEDI0680 is described, for example, in U.S. Pat. No. 8,609,089B2.

In some embodiments, the anti-PD-1 antibody is BGB-A317, which is a humanized monoclonal antibody. BGB-A317 is described in U.S. Publ. No. 2015/0079109.

In some embodiments, a PD-1 antagonist is AMP-224, which is a B7-DC Fc fusion protein. AMP-224 is discussed in U.S. Publ. No. 2013/0017199.

In some embodiments, the subject has been treated with a monoclonal antibody that binds to PD-L1 prior to treatment with OX40L, IL-23 and IL-36γ encoding mRNAs. In some embodiments, the subject has been treated with an anti-PD-L1 monoclonal antibody therapy simultaneously with or subsequent to treatment with OX40L, IL-23 and IL-36γ encoding mRNAs. In some embodiments, the anti-PD-L1 monoclonal antibody therapy comprises Durvalumab, Avelumab, MEDI473, BMS-936559, Atezolizumab, or any combination thereof.

Durvalumab is a human IgG1, kappa mAb that blocks the interaction of PD-L1 (but not PD-L2) with PD-1 on T-cells and CD80 proteins on immune cells. Durvalumab is developed for use in the treatment of cancer. The proposed mechanism of action for durvalumab is interference in the interaction of PD-L1 with PD-1 and CD80. Blockade of PD-L1/PD-1 and PD-L1/CD80 interactions releases the inhibition of immune responses, including those that may result in tumor elimination. Durvalumab is engineered to reduce antibody-dependent cellular cytotoxicity and complement-dependent cytotoxicity. Thus, durvalumab is expected to stimulate anti-tumor immune response by binding to PD-L1 and shifting the balance toward anti-tumor response.

In some embodiments, the anti-PD-L1 antibody useful for the disclosure is MSB0010718C (also called Avelumab; See US 2014/0341917) or BMS-936559 (formerly 12A4 or MDX-1105) (see, e.g., U.S. Pat. No. 7,943,743; WO 2013/173223). In other embodiments, the anti-PD-L1 antibody is MPDL3280A (also known as RG7446) (see, e.g., Herbst et al. (2013) J Clin Oncol 31(suppl):3000. Abstract; U.S. Pat. No. 8,217,149), MEDI14736 (also called Durvalumab; Khleif (2013) In: Proceedings from the European Cancer Congress 2013; Sep. 27-Oct. 1, 2013; Amsterdam, The Netherlands).

In some embodiments, the subject has been treated with a CTLA-4 antagonist prior to treatment with OX40L, IL-23 and IL-36γ encoding mRNAs. In some embodiments, the subject has been previously treated with a monoclonal antibody that binds to CTLA-4 prior to treatment with OX40L, IL-23 and IL-36γ encoding mRNAs. In some embodiments, the subject has been treated with an anti-CTLA-4 monoclonal antibody simultaneously with or subsequent to treatment with OX40L, IL-23 and IL-36γ encoding mRNAs. In other aspects, the anti-CTLA-4 antibody therapy comprises Ipilimumab or Tremelimumab.

An exemplary clinical anti-CTLA-4 antibody is the human mAb 10D1 (now known as ipilimumab and marketed as YERVOY®) as disclosed in U.S. Pat. No. 6,984,720. Another anti-CTLA-4 antibody useful for the present methods is tremelimumab (also known as CP-675,206). Tremelimumab is human IgG2 monoclonal anti-CTLA-4 antibody. Tremelimumab is described in WO/2012/122444, U.S. Publ. No. 2012/263677, or WO Publ. No. 2007/113648 A2.

In a preferred embodiment, an mRNA therapeutic of the invention is administered to a subject having a solid tumor malignancy or lymphoma in combination with a checkpoint inhibitor.

In some embodiments, an mRNA therapeutic agent described herein is administered to a subject having a solid tumor malignancy or lymphoma in combination with a checkpoint inhibitor, wherein the checkpoint inhibitor is administered intravenously. In some embodiments, the checkpoint inhibitor is administered once every 4 weeks for a duration of time. In some embodiments, the mRNA therapeutic agent is administered at a dose for a first dosing cycle comprising administration of the dose at a dosing interval of once every two weeks for a duration of time, and at least one subsequent dosing cycle comprising administration of the dose at a dosing interval of once every 4 weeks for a duration of time, and the checkpoint inhibitor is administered once every 4 weeks for a duration of time.

In some embodiments, the checkpoint inhibitor is administered once every 4 weeks for 28-42 days. In some embodiments, the mRNA therapeutic agent is administered at a dose for a first dosing cycle comprising administration of the dose at a dosing interval of once every two weeks for 28-42 days, and at least one subsequent dosing cycle comprising administration of the dose at a dosing interval of once every 4 weeks for 28-42 days, and the checkpoint inhibitor is administered once every 4 weeks for a specified period of time.

In some embodiments, the checkpoint inhibitor is administered once every 4 weeks for 28 days. In some embodiments, the mRNA therapeutic agent is administered at a dose for a first dosing cycle comprising administration of the dose at a dosing interval of once every two weeks for 28 days, and at least one subsequent dosing cycle comprising administration of the dose at a dosing interval of once every 4 weeks for 28 days, and the checkpoint inhibitor is administered once every 4 weeks for a specified period of time.

In some embodiments, the checkpoint inhibitor continues to be administered once every 4 weeks even after administration of the mRNA therapeutic agent has stopped.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

### Exemplary Cancers

The mRNA therapeutic described herein, alone or in combination, are useful for treating solid tumor malignancies or lymphomas. In some embodiments, the solid tumor malignancy or lymphoma is advanced and/or metastatic.

In some embodiments, the solid tumor malignancy is triple negative breast cancer (TNBC). TNBC makes up between 15-20% of all diagnosed breast cancer cases. TNBC is defined by the lack of protein expression of estrogen receptor (ER), progesterone receptor (PgR) and the absence of human epidermal growth factor receptor 2 (HER2) protein over-expression. TNBC is associated with poor prognosis consisting of low five-year survival rates and high recurrence.

In some embodiments, the solid tumor malignancy is head and neck squamous cell carcinoma (HNSCC). HNSCC accounts for almost 90% of cancers involving the upper aerodigestive tract. Five-year survival rates for HNSCC are low and have not improved in several decades. Patients with this disease experience sever morbidity including disfigurement, speech, swallowing and breathing problems,

In some embodiments, the solid tumor malignancy is melanoma. Melanoma is a malignant tumor of melanocytes, has the ability to spread to other organs, and is among the most commonly occurring cancers. Melanoma carcinomas include superficial spreading melanoma, nodular melanoma, acral lentiginous melanoma, and lentigo maligna. The overall 5-year survival for melanoma is 91%. However, if distal metastasis occurs, cure rates are <15%. In some embodiments, the melanoma is refractory to CPI. In some embodiments, the melanoma is primary refractory to CPI. In some embodiments, the melanoma is secondary acquired resistance to CPI. In some embodiments, the melanoma has not received anti-cancer treatment prior to administration of mRNAs encoding human OX40L, IL-23, and IL-36γ polypeptides, and is referred to herein as “neoadjuvant melanoma.”

In some embodiments, the solid tumor malignancy is bladder cancer. Bladder cancer is the most common urinary cancer, and the most common type of bladder cancer (about 90%) is transitional cell carcinoma (TCC), which derived from the urothelium (the cellular lining of the urethral system). TCC can be classified as either superficial, meaning that tumor involvement is limited to the mucosal or submucosal layer of the urothelium, or muscle invasive. Other types of bladder cancers include squamous cell carcinomas, adenocarcinomas, sarcomas and small cell carcinomas. Accordingly, the term “bladder cancer” refers to cancer arising from cells in the urinary bladder and is used to include TCC (also referred to as “urothelial cancer”), squamous cell carcinoma, adenocarcinoma, small cell carcinoma, and sarcoma. TCC typically includes two-sub types, papillary carcinomas in which the tumors grow in slender, finger-like projections from the inner surface of the bladder toward the hollow center, and flat carcinomas, in which tumor do not grow toward the hollow part of the bladder. Squamous cell carcinoma and adenocarcinoma are less common types of bladder cancer, and small cell carcinoma and sarcoma are relatively rare. In some embodiments, the solid tumor malignancy is a urothelial cancer. In some embodiments, the urothelial cancer responds to CPI antibody therapy. In some embodiments, the solid tumor malignancy is squamous cell carcinoma bladder cancer. In some embodiments, squamous cell carcinoma bladder cancer has negative or low expression of PD-L1. In some embodiments, squamous cell carcinoma bladder cancer is unresponsive to CPI antibody therapy. In some embodiments, the mRNA therapeutic agent disclosed herein increases expression of PD-L1 in squamous cell carcinoma bladder cancer such that it is responsive to CPI antibody therapy. In some embodiments, the solid tumor malignancy is a bladder adenocarcinoma.

In some embodiments, the solid tumor malignancy is non-small cell lung carcinoma (NSCLC). NSCLC is any type of epithelial lung cancer other than small-cell lung carcinoma (SCLC), and accounts for about 80-85% of all lung cancers. The main subtypes of NSCLC are adenocarcinoma, squamous cell carcinoma, and large cell carcinoma. Generally, NSCLCs are less sensitive to chemotherapy and radiation therapy compared to SCLC. In some embodiments, the NSCLC is refractory to CPI. In some embodiments, the NSCLC is primary refractory to CPI. In some embodiments, the NSCLC is secondary acquired resistance to CPI.

In some embodiments, the lymphoma is Non-Hodgkin lymphoma (NHL). NHLs are cancers of B, T or natural killer lymphocytes. The two most common types of NHL, follicular lymphoma (FL) and diffuse large B-cell lymphoma (DLBCL), together comprise 60% of new B-cell NHL diagnoses each year in North America. FL is an indolent and typically incurable disease characterized by clinical and genetic heterogeneity. DLBCL is aggressive and likewise heterogeneous, comprising at least two distinct subtypes that respond differently to standard treatments.

In some embodiments, the solid tumor malignancy or lymphoma does not express PD-L1 or expresses low levels of PD-L1. In some embodiments, the lack of expression or low expression of PD-L1 indicates lack of T cell infiltration into the tumor. In some embodiments, cancer that does not express or has low expression of PD-L1 does not respond to CPI antibody therapy. In some embodiments, the mRNA therapeutic agent described herein induces or increases PD-L1 expression in the solid tumor malignancy or lymphoma. In some embodiments, the solid tumor malignancy or lymphoma is response to CPI antibody therapy after administration with the mRNA therapeutic agent described herein.

In some embodiments, the patient with a solid tumor malignancy or lymphoma has not previously been treated or exposed to an anti-cancer treatment prior to administration with the mRNA therapeutic agent described herein. In some embodiments, the patient with a solid tumor malignancy or lymphoma is primary refractory to CPI. In some embodiments, CPI-primary refractory cancer occurs when a demonstration of progression of cancer has been observed in the patient after exposure to CPI. In some embodiments, the patient with a solid tumor malignancy or lymphoma is acquired secondary resistance to CPI. In some embodiments, CPI-acquired secondary resistance occurs when a demonstration of progression of cancer has been observed in the patient after a confirmed objective response or prolonged stable disease after exposure to CPI followed by disease progression in the setting of ongoing treatment with CPI. In some embodiments, the patient has any one of the cancers described herein which is CPI-refractory.

Compositions Comprising mRNAs Encoding OX40L, IL-23 and IL-36γ

The present disclosure provides compositions, including LNP encapsulated mRNA therapeutic agents for the treatment of cancer. In one embodiment, the compositions comprise, in a single formulation, at least three mRNAs, each of the compositions selected from a first mRNA encoding OX40L, a second mRNA encoding IL-23 and/or a third mRNA encoding IL-36γ. Accordingly, the present disclosure provides, for example, (i) a first mRNA encoding a first protein comprising an OX40L polypeptide, (ii) a second mRNA encoding a second protein comprising an IL-23 polypeptide, and (iii) a third mRNA encoding a third protein comprising an IL-36γ polypeptide, wherein the first mRNA, the second mRNA, and the third mRNA are used in various combinations. In one aspect, the composition comprises the first mRNA, the second mRNA, and the third mRNA.

In some aspects of the methods disclosed herein, the first mRNA encoding a human OX40L polypeptide, the second mRNA encoding a human IL-23 polypeptide, and the third mRNA encoding a human IL-36γ polypeptide, are formulated, e.g., encapsulated in an LNP for in vivo delivery, e.g., intratumoral injection. In some embodiments, the first mRNA, the second mRNA, and the third mRNA are co-formulated (e.g., encapsulated in an LNP) at varying weight ratios, for example, with equivalent amounts (by weight) of each mRNA or with any one of the mRNAs present at 2, 3, 4, 5, 6, 7, 8, 9, 10, 11, 12, 13, 14, 15, 16, 17, 18, 19, 20, 25, 30, 35, 40, 45, or 50 times the amount (by weight) of the other mRNA. In one embodiment, the IL-23:IL-36γ:OX40L mRNAs are co-formulated at a weight (mass) ratio such that the IL-23 and OX40L mRNAs are at about equal amounts and the IL-36γ mRNA is present at a higher weight (mass) amount, such as 1.5, 2.0, 2.5, 3.0, 3.5, 4.0, 4.5 or 5.0 times greater weight (mass) amount. In one particular embodiment, the IL-23:IL-36γ:OX40L mRNAs are co-formulated at a weight (mass) ratio of 1:2:1. In used herein, the mass ratio can also be referred to by reference to a composition comprising mRNAs encoding OX40L:IL-23:IL-36γ formulated at a weight (mass) ratio of 1:1:2.

In other embodiments, the IL-23:IL-36γ:OX40L mRNAs are co-formulated at a weight (mass) ratio of 1:1:1, 2:1:1, 1:2:1, 1:1:2, 3:1:1, 1:3:1, 1:1:3, 4:1:1, 1:4:1, 1:1:4, 5:1:1, 1:5:1, 1:1:5, 6:1:1, 1:6:1, 1:1:6, 7:1:1, 1:7:1, 1:1:8, 9:1:1, 1:9:1, 1:1:9, 10:1:1, 1:10:1, 1:1:10, 11:1:1, 1:11:1, 1:1:11, 12:1:1, 1:12:1, 1:1:12, 13:1:1, 1:13:1, 1:1:13, 14:1:1, 1:14:1, 1:1:14, 15:1:1, 1:15:1, 1:1:15, 16:1:1, 1:16:1, 1:1:16, 17:1:1, 1:17:1, 1:1:17, 18:1:1, 1:18:1, 1:1:18, 19:1:1, 1:19:1, 1:1:19, 20:1:1, 1:20:1, 1:1:20, 25:1:1, 1:25:1, 1:1:25, 30:1:1, 1:30:1, 1:1:30, 35:1:1, 1:35:1, 1:1:35, 40:1:1, 1:40:1, 1:1:40, 45:1:1, 1:45:1, 1:1:45, 50:1:1, 1:50:1, or 1:1:50. In other embodiments, each of the three mRNAs can be present in the co-formulation at a different weight. By way of example only, the IL-23:IL-36γ:OX40L mRNAs can be co-formulated at a weight (mass) ratio of 1:2:3, 1:3:2, 2:1:3, 2:3:1, 3:1:2, or 3:2:1; or alternative at a weight (mass) ratio of 1:3:5, 1:5:3, 3:5:1, 3:1:5, 5:1:3, or 5:3:1; or alternative at a weight (mass) ratio of 1:5:10, 1:10:5, 5:1:10, 5:10:1, 10:1:5, or 10:5:1. Ina particular embodiment, (i) a first mRNA encoding a first protein comprising an OX40L polypeptide (e.g., SEQ ID NO: 1), (ii) a second mRNA encoding a second protein comprising an IL-36-γ polypeptide (e.g., SEQ ID NO: 27), and (iii) a third polypeptide encoding a third protein comprising an IL-23 polypeptide (e.g., SEQ ID NO: 24), are formulated in a weight (mass) ratio of 1:2:1. While this is one exemplary formulation, the skilled artisan will readily appreciate that amounts of any one of the three constituents outside of this ratio may also provide formulations which are suitable for use in any of the methods disclosed herein.

The mRNA co-formulation can be administered as a single dose or as multiple doses. Co-formulations with varying weight (mass) ratios, e.g., co-formulation #1 in which the first mRNA, the second mRNA, and the third mRNA are present at 1:2:1 w/w and co-formulation #2 in which the first mRNA, the second mRNA, and the third mRNA are present at 1:1:2 w/w, can each be administered once or multiple times sequentially, concurrently, or simultaneously.

In one embodiment, the 1:2:1 co-formulation of (i) a first mRNA encoding a first protein comprising an OX40L polypeptide (e.g., SEQ ID NO: 1), (ii) a second mRNA encoding a second protein comprising an IL-36-γ polypeptide (e.g., SEQ ID NO: 27), and (iii) a third polypeptide encoding a third protein comprising an IL-23 polypeptide (e.g., SEQ ID NO: 24), is administered as a single dose or as multiple doses.

In one embodiment, the disclosure provides an LNP encapsulated mRNA therapeutic agent comprising three mRNA drug substances, wherein the first, second, and third mRNAs are formulated in the lipid nanoparticle at a mass ratio of OX40L:IL-23:IL-36γ of 1:1:2, and wherein:

(i) the first mRNA encoding a human OX40L polypeptide comprises an ORF comprising a nucleotide sequence at least 80% identical to the nucleotide sequence set forth in SEQ ID NO: 4 or comprising, the nucleotide sequence set forth in SEQ ID NO: 4;

(ii) the second mRNA encoding a human IL-23 polypeptide comprises an ORF comprising a nucleotide sequence at least 80% identical to the nucleotide sequence set forth in SEQ ID NO: 25 or comprises the nucleotide sequence set forth in SEQ ID NO: 25; and

(iii) the third mRNA encoding a human IL-36γ polypeptide comprises an ORF comprising a nucleotide sequence at least 80% identical to the nucleotide sequence set forth in SEQ ID NO: 28 or comprises the nucleotide sequence set forth in SEQ ID NO: 28.

In one embodiment, the disclosure provides an LNP encapsulated mRNA therapeutic agent comprising three mRNA drug substances, wherein the first, second, and third mRNAs are formulated in the lipid nanoparticle at a mass ratio of OX40L:IL-23:IL-36γ of 1:1:2, and wherein:

(i) the first mRNA encoding the human OX40L polypeptide comprises a nucleotide sequence at least 90% identical to the nucleotide sequence set forth in SEQ ID NO: 5 or comprises the nucleotide sequence set forth in SEQ ID NO: 5;

(ii) the second mRNA encoding a human IL-23 polypeptide comprises a nucleotide sequence at least 90% identical to the nucleotide sequence set forth in SEQ ID NO: 26 or comprises the nucleotide sequence set forth in SEQ ID NO: 26; and

(iii) the third mRNA encoding a human IL-36γ polypeptide comprises a nucleotide sequence at least 90% identical to the nucleotide sequence set forth in SEQ ID NO: 29 or comprises the nucleotide sequence set forth in SEQ ID NO: 29.

It is to be understood that the term “combinations of the disclosure” is not limited to the physical combination of a first mRNA, a second mRNA, and/or a third mRNA, but also encompasses the separate administration of these mRNAs concurrently or sequentially.

mRNAs Encoding OX40L

The mRNAs of the present disclosure encode an OX40L polypeptide. OX40L, the ligand for OX40 (CD134), is a homo-trimeric transmembrane protein normally expressed on antigen-presenting cells upon immune stimulation (Mallett et al., (1990) EMBO J 9(4):1063-1068).

Binding of OX40 and OX40L in the presence of a recognized antigen (e.g., a tumor antigen) promotes the expansion of CD4+ and CD8+ T cells and enhances memory responses while inhibiting regulatory T cells. Expression of OX40L by tumor cells, or other cells presenting tumor antigens is known to induce cell-mediated immune responses with systemic anti-tumor effects. OX40L has also been designated CD252 (cluster of differentiation 252), tumor necrosis factor (ligand) superfamily, member 4, tax-transcriptionally activated glycoprotein 1, TXGP1, or gp34. Human OX40L is 183 amino acids in length and contains three domains: a cytoplasmic domain of amino acids 1-23; a transmembrane domain of amino acids 24-50, and an extracellular domain of amino acids 51-183.

Human OX40L was first identified on the surface of human lymphocytes infected with human T-cell leukemia virus type-I (HTLV-I) by Tanaka et al. (Tanaka et al., International Journal of Cancer (1985), 36(5):549-55). Human OX40L is a 34 kDa glycosylated type II transmembrane protein that exists on the surface of cells as a trimer. OX40L comprises a cytoplasmic domain (amino acids 1-23), a transmembrane domain (amino acids 24-50) and an extracellular domain (amino acids 51-183). OX40L is also referred to as Tumor Necrosis Factor Superfamily (ligand) Member 4 (TNFSF4), CD252, CD134L, Tax-Transcriptionally Activated Glycoprotein 1 (TXGP1), Glycoprotein 34 (GP34), and ACT-4-L.

In some embodiments, a composition suitable for use in the methods of the disclosure comprises an mRNA encoding a mammalian OX40L polypeptide. In some embodiments, the mammalian OX40L polypeptide is a human OX40L polypeptide. In some embodiments, the OX40L polypeptide comprises an amino acid sequence set forth in SEQ ID NOs: 1-3.

In some embodiments, the mRNA encoding a human OX40L polypeptide encodes a human OX40L polypeptide comprising an amino acid sequence at least 50%, at least 60%, at least 70%, at least 80%, at least 85%, at least 90%, at least 95%, at least 98%, at least 99%, or 100% identical to an amino acid sequence set forth in SEQ ID NOs: 1-3 or an amino acid sequence encoded by a nucleotide sequence set forth in SEQ ID NOs: 4-10, wherein the human OX40L polypeptide is capable of binding to an OX40 receptor. In some embodiments, the mRNA encoding a human OX40L polypeptide encodes a human OX40L polypeptide comprising an amino acid sequence at least 50%, at least 60%, at least 70%, at least 80%, at least 85%, at least 90%, at least 95%, at least 98%, at least 99%, or 100% identical to SEQ ID NO: 1 and is capable of binding to an OX40 receptor. In some embodiments, the mRNA encoding a human OX40L polypeptide encodes a human OX40L polypeptide that consists essentially of SEQ ID NO: 1 and is capable of binding to an OX40 receptor.

In certain embodiments, the mRNA encoding a human OX40L polypeptide encodes a human OX40L polypeptide comprising an amino acid sequence set forth in SEQ ID NOs: 1-3, optionally with one or more conservative substitutions, wherein the conservative substitutions do not significantly affect the binding activity of the OX40L polypeptide to its receptor, i.e., the OX40L polypeptide binds to the OX40 receptor after the substitutions. In some embodiments, the mRNA encoding a human OX40L polypeptide encodes a human OX40L polypeptide comprising an amino acid sequence having at least 80%, at least 85%, at least 90%, at least 95%, at least 99% or 100% identity to any one of the amino acid sequences set forth in SEQ ID NOs: 1-3.

In other embodiments, an mRNA encoding a human OX40L polypeptide comprises a nucleotide sequence at least 50%, at least 60%, at least 70%, at least 80%, at least 85%, at least 90%, at least 95%, at least 98%, at least 99%, or 100% identical to any one of the nucleic acid sequences set forth in SEQ ID NOs: 4-10. In some embodiments, the mRNA encoding a human OX40L polypeptide comprises an open reading frame comprising a nucleotide sequence selected from any one of SEQ ID NOs: 4 and 8-10. In some embodiments, the mRNA encoding a human OX40L polypeptide comprises an open reading frame comprising a nucleotide sequence having at least 80%, at least 85%, at least 90%, at least 95%, at least 99%, or 100% identity to a nucleotide sequence selected from any one of SEQ ID NOs: 4 and 8-10. In some embodiments, the mRNA encoding a human OX40L polypeptide comprises an open reading frame comprising the nucleotide sequence set forth in SEQ ID NO: 4. In some embodiments, the mRNA encoding a human OX40L polypeptide comprises an open reading frame comprising a nucleotide sequence having at least 80%, at least 85%, at least 90%, at least 95%, at least 99%, or 100% identity to the nucleotide sequence set forth in SEQ ID NO: 4. In some embodiments, the mRNA encoding a human OX40L polypeptide comprises an open reading frame comprising the nucleotide sequence set forth in SEQ ID NO: 8. In some embodiments, the mRNA encoding a human OX40L polypeptide comprises an open reading frame comprising a nucleotide sequence having at least 80%, at least 85%, at least 90%, at least 95%, at least 99%, or 100% identity to the nucleotide sequence set forth in SEQ ID NO: 8. In some embodiments, the mRNA encoding a human OX40L polypeptide comprises an open reading frame comprising the nucleotide sequence set forth in SEQ ID NO: 9. In some embodiments, the mRNA encoding a human OX40L polypeptide comprises an open reading frame comprising a nucleotide sequence having at least 80%, at least 85%, at least 90%, at least 95%, at least 99%, or 100% identity to the nucleotide sequence set forth in SEQ ID NO: 9. In some embodiments, the mRNA encoding a human OX40L polypeptide comprises an open reading frame comprising the nucleotide sequence set forth in SEQ ID NO: 10. In some embodiments, the mRNA encoding a human OX40L polypeptide comprises an open reading frame comprising a nucleotide sequence having at least 80%, at least 85%, at least 90%, at least 95%, at least 99%, or 100% identity to the nucleotide sequence set forth in SEQ ID NO: 10.

In some embodiments, the mRNA useful for the methods and compositions described herein comprises an open reading frame encoding an extracellular domain of OX40L. In other embodiments, the mRNA comprises an open reading frame encoding a cytoplasmic domain of OX40L. In some embodiments, the mRNA comprises an open reading frame encoding a transmembrane domain of OX40L. In certain embodiments, the mRNA comprises an open reading frame encoding an extracellular domain of OX40L and a transmembrane domain of OX40L. In other embodiments, the mRNA comprises an open reading frame encoding an extracellular domain of OX40L and a cytoplasmic domain of OX40L. In yet other embodiments, the mRNA comprises an open reading frame encoding an extracellular domain of OX40L, a transmembrane of OX40L, and a cytoplasmic domain of OX40L.

A person of skill in the art would understand that in addition to the native signal sequences and propeptide sequences implicitly disclosed in SEQ ID NOs: 1-10 (sequences present in the precursor form and absent in the mature corresponding form) and non-native signal peptides, other signal sequences can be used. Accordingly, references to OX40L polypeptide or mRNA according to SEQ ID NOs: 1-10 encompass variants in which an alternative signal peptide (or encoding sequence) known in the art has been attached to said OX40L polypeptide (or mRNA). It is also understood that references to the sequences disclosed in SEQ ID NOs: 1-10 through the application are equally applicable and encompass orthologs and functional variants (for example polymorphic variants) and isoforms of those sequences known in the art at the time the application was filed.

In some embodiments, the OX40L encoding mRNA comprises an open reading frame encoding OX40L, a 3′UTR, and a 5′UTR. In some embodiments, the OX40L encoding mRNA comprises an open reading frame encoding OX40L, a 3′UTR, a 5′UTR, and a poly-A tail. In some embodiments, the OX40L encoding mRNA comprises an open reading frame encoding OX40L, a 3′UTR, a 5′UTR, a poly-A tail and a 5′cap.

In some embodiments, the OX40L encoding mRNA comprises (i) a 5′UTR comprising the nucleotide sequence set forth in SEQ ID NO: 15; (ii) an open reading frame encoding OX40L comprising the nucleotide sequence set forth in SEQ ID NO: 4; and (iii) a 3′UTR comprising the nucleotide sequence set forth in SEQ ID NO: 17. In some embodiments, the OX40L encoding mRNA comprises (i) a 5′UTR comprising the nucleotide sequence set forth in SEQ ID NO: 16; (ii) an open reading frame encoding OX40L comprising the nucleotide sequence set forth in SEQ ID NO: 4; and (iii) a 3′UTR comprising the nucleotide sequence set forth in SEQ ID NO: 17.

In some embodiments, the OX40L encoding mRNA comprises the nucleotide sequence set forth in SEQ ID NO: 5. In some embodiments, the OX40L encoding mRNA comprises a nucleotide sequence having at least 80%, 85%, 90%, 95%, 96%, 97%, 98% or 99% identity to the nucleotide sequence set forth in SEQ ID NO: 5.

mRNA Encoding IL-23

The mRNAs of the present disclosure encode an IL-23 polypeptide. IL-23 is a pro-inflammatory cytokine that plays an important role in innate and adaptive immunity. Croxford et al. (2012) Eur. J. Immunol. 42:2263-2273. IL-23 functions primarily as a 60 kDa heterodimeric protein consisting of disulfide-linked p19 and p40 subunits. IL-23 is structurally and functionally similar to the pro-inflammatory cytokine IL-12. IL-23 contains the same p40 subunit as IL-12, but includes the p19 subunit rather than IL-12's p35. Oppman et al. (2000) Immunity 13:715-725. The precursor form of the p19 subunit (NCBI Reference Sequence: NP_057668; NM_016584; Uniprot: Q9NPF7; also referred to as IL-23A and IL-23 subunit alpha) is 189 amino acids in length, while its mature form is 170 amino acids long. The precursor form of the p40 subunit (NCBI Reference Sequence: NM_002187; Uniprot: P29460; also referred to as IL-12B, natural killer cell stimulatory factor 2, and cytotoxic lymphocyte maturation factor 2) is 328 amino acids in length, while its mature form is 306 amino acids long.

Many different immune cells, including dendritic cells and macrophages, produce IL-23 upon antigenic stimuli. One difference between IL-12 and IL-23 is that IL-12 is associated with the development and activity of Th1 T cell populations, while IL-23 is associated with the development and activity of Th17 T cell populations. See Vignali et al. (2014) Nat. Immunol. 13:722-728.

Although some early studies implicated IL-23 for anti-tumor therapy (Belladonna et al. (2002) J. Immunol. 168:5448-5454), more recent studies indicate a potential pro-tumorigenic function for IL-23. See, e.g., Croxford et al. (2012) Eur. J. Immunol. 42:2263-2273; Langowski et al. (2007) Trends Immunol. 28:207-212; Langowski et al. (2006) Nature 442:461-465; Teng et al. (2010) Proc. Natl. Acad. Sci. USA 107:8328-8333; Teng et al. (2012) Cancer Res. 72:3987-3996. Langowski (2006) observed an increase of IL-23 in human tumors. See also Ngiow et al. (2013) Trends Immunol. 34:548-555; Wilke et al. (2011) Carcinogenesis 32:643-649; Xu et al. (2010) Clin. Dev. Immunol. 2010. For example, Wang et al. (2015) Clin. Exp. Rheumatol. 33 (Suppl. 92): S87-S90 teaches that elevated expression of IL-23 has a pathogenic function in cancer. IL-23 has a causal role in tumor development and progression and has been linked to adverse prognostic outcome and rapid progression to metastatic disease, suggesting that inhibition of IL-23 expression may be useful for therapy and prevention of cancer, particularly colorectal cancer. Teng et al. (2015) Nature Medicine 21: 719-29 teaches that IL-23 indirectly or directly promotes tumorigenesis, growth, and metastasis, and indicates that inhibition of IL-23 expression could be used for therapy and prevention of cancer.

As used in the present disclosure, the term “IL-23 polypeptide” refers to, e.g., a IL-12p40 subunit of IL-23, to an IL-23p19 subunit of IL-23, or to a fusion protein comprising an IL-12p40 subunit polypeptide and an IL-23p19 subunit polypeptide. In some aspects, the fusion protein comprises from N-terminus to C-terminus:

(a) an IL-12p40 subunit comprising the IL-12p40 signal peptide, a peptide linker, and a mature IL-23p19 subunit, or
(b) an IL-23p19 subunit comprising the IL-23p19 signal peptide, a peptide linker, and a mature IL-12p40.

In one particular aspect, the IL-23 polypeptide comprises, consists of, or consists essentially of a human IL-23 polypeptide of SEQ ID NO: 24 (e.g., a precursor or mature IL-12p40 or IL-23p19) or a combination thereon. In one particular aspect, the mRNA encoding the IL-23 polypeptide comprises, consists of, or consists essentially of an IL-23-encoding mRNA of SEQ ID NO: 25. In one particular aspect, the mRNA encoding the IL-23 polypeptide comprises, consists of, or consists essentially of an IL-23-encoding mRNA of SEQ ID NO: 26.

In some embodiments, the IL-23 polypeptide comprises an amino acid sequence at least 50%, at least 60%, at least 70%, at least 80%, at least 85%, at least 90%, at least 95%, at least 98%, at least 99%, or 100% identical to an IL-23 amino acid sequence listed in SEQ ID NO: 24 or an amino acid sequence encoded by a nucleotide sequence of SEQ ID NO: 25, wherein the IL-23 polypeptide has at least 10% of the activity (e.g., binding to its receptor) of the corresponding wild type IL-23 polypeptide. In a particular embodiment, the IL-23 polypeptide comprises an amino acid sequence at least 50%, at least 60%, at least 70%, at least 80%, at least 85%, at least 90%, at least 95%, at least 98%, at least 99%, or 100% identical to SEQ ID NO: 24 and has at least 10% of the activity (e.g., binding to its receptor) of the corresponding wild type IL-23 polypeptide. In another particular embodiment, the IL-23 polypeptide consists essentially of SEQ ID NO: 24 and has at least 10% of the activity (e.g., binding to its receptor) of the corresponding wild type IL-23 polypeptide.

In other embodiments, the IL-23 polypeptide encoded by a mRNA of the disclosure comprises an amino acid sequence of SEQ ID NO: 24 with one or more conservative substitutions, wherein the conservative substitutions do not significantly affect the binding activity of the IL-23 polypeptide to its receptor, i.e., the IL-23 polypeptide binds to the IL-23 receptor after the substitutions.

In some embodiments, a nucleotide sequence (i.e., mRNA) encoding an IL-23 polypeptide comprises a sequence at least 50%, at least 60%, at least 70%, at least 80%, at least 85%, at least 90%, at least 95%, at least 98%, at least 99%, or 100% identical to an IL-23 polypeptide encoding nucleic acid sequence listed in Table 1. In a particular embodiment, the nucleotide sequence (i.e., mRNA) encoding an IL-23 polypeptide comprises a sequence at least 50%, at least 60%, at least 70%, at least 80%, at least 85%, at least 90%, at least 95%, at least 98%, at least 99%, or 100% identical to SEQ ID NO: 25 or SEQ ID NO: 26. In another particular embodiment, the nucleotide sequence (i.e., mRNA) encoding an IL-23 polypeptide consists essentially SEQ ID NO: 25 or SEQ ID NO: 26. It should be understood that the nucleotide sequence (i.e., mRNA, e.g., SEQ ID NO: 25) encoding an IL-23 polypeptide open reading frame (ORF) can be one element within a larger construct, e.g., further including a 5′ terminal cap, 5′UTR, 3′UTR, and/or polyA tail.

In some embodiments, the IL-23 polypeptide comprises an IL-12p40 subunit comprising an amino acid sequence at least about 50%, at least about 55%, at least about 60%, at least about 65%, at least about 70%, at least about 75%, at least about 80%, at least about 85%, at least about 90%, at least about 95%, at least about 99%, or 100% to an IL-23 polypeptide sequence of SEQ ID NO: 24, wherein the amino acid sequence is capable of binding to an IL-23p19 subunit and forming IL-23, which has an IL-23 activity.

In some embodiments, the IL-12p40 subunit is encoded by a nucleic acid sequence at least about 70%, at least about 75%, at least about 80%, at least about 85%, at least about 90%, at least about 95%, at least 99%, or 100% identical to an IL-23 polypeptide encoding SEQ ID NO: 24.

In some embodiments, the IL-23 polypeptide comprises an IL-23p19 subunit comprising an amino acid sequence at least about 50%, at least about 55%, at least about 60%, at least about 65%, at least about 70%, at least about 75%, at least about 80%, at least about 85%, at least about 90%, at least about 95%, at least about 99%, or 100% identical to an IL-23 polypeptide sequence listed of SEQ ID NO: 24, wherein the amino acid sequence is capable of binding to an IL-12p40 subunit and forming IL-23, which has an IL-23 activity.

In some embodiments, the IL-23p19 subunit is encoded by a nucleic acid sequence at least about 70%, at least about 75%, at least about 80%, at least about 85%, at least about 90%, at least about 95%, at least about 99%, or 100% identical to a IL-23 polypeptide encoding SEQ ID NO: 25 or SEQ ID NO: 26.

In some embodiments, the IL-12p40 subunit and the IL-23p19 subunit of the IL-23 protein are on a single polypeptide chain or two different chains. In some embodiments, the IL-12p40 subunit and the IL-23p19 subunit are fused by a linker. In some embodiments, the IL-12p40 subunit comprises a signal peptide. In some embodiments, the IL-23p19 subunit comprises a signal peptide. In some embodiments, the IL-12p40 subunit is a mature IL-12p40 (i.e., it does not comprise a signal peptide). In some embodiments, the IL-23p19 subunit is a mature IL-23p19 (i.e., it does not comprise a signal peptide). In some embodiments, the IL-12p40 subunit comprises a non-native signal peptide. In some aspects, the IL-23p19 subunit comprises a non-native signal peptide.

In some embodiments, the IL-23 is a fusion polypeptide comprising an IL-12p40 subunit and an IL-23p19 subunit according to any of the following alternative formulas:

[signal peptide 1]-[IL-12p40]-[linker]-[IL-23p19]

[signal peptide 2]-[IL-23p19]-[linker]-[IL-12p40]

wherein [signal peptide 1] can be an IL-12p40 signal peptide or a non-native signal peptide, [signal peptide 2] can be an IL-23p19 signal peptide or a non-native signal peptide, [IL-12p40] is a mature IL-12p40, [IL-23p19] is a mature IL-23p29, and [linker] is a peptide linker.
mRNA Encoding IL-36γ

The mRNAs of the present disclosure encode an IL-36γ polypeptide. IL-36γ is a member of the Interleukin-1 family of cytokines. Like other members of the interleukin-1 family of cytokines, IL-36γ requires N-terminal cleavage for full bioactivity. IL-36γ does not have a signal sequence and, therefore, is not secreted through the endoplasmic reticulum Golgi pathway. (See Gresnigt and van de Veerdonk (2013) Seminars in Immunology 25:458-465). It is unclear how IL-36γ is released from cells to act on, e.g., immune cells, other epithelial cells, and fibroblasts (Gabay et al. (2015) Journal of Leukocyte Biology 97:645-652). In exemplary aspects of the invention, a mRNA encoding IL-36, e.g., IL-36γ, includes a sequence encoding a heterologous signal peptide. Without being bound in theory, it is believed that mRNAs encoding such “engineered” signal peptide-interleukin chimeric proteins provide for the generation of active protein when expressed in vivo, in the absence of inflammasome activation.

In one particular aspect, the IL-36γ polypeptide comprises, consists of, or consists essentially of an IL-36γ polypeptide of Table 1. In one particular aspect, the mRNA encoding the IL-36γ-polypeptide comprises, consists of, or consists essentially of an IL-36γ-encoding mRNA of SEQ ID NO: 28. In one particular aspect, the mRNA encoding the IL-36γ-polypeptide comprises, consists of, or consists essentially of an IL-36γ-encoding mRNA of SEQ ID NO: 29.

In some embodiments, the IL-36γ polypeptide comprises an amino acid sequence at least 50%, at least 60%, at least 70%, at least 80%, at least 85%, at least 90%, at least 95%, at least 98%, at least 99%, or 100% identical to an IL-36γ amino acid sequence of SEQ ID NO: 27 or an amino acid sequence encoded by a nucleotide sequence listed SEQ ID NO: 28 or SEQ ID NO: 29, wherein the IL-36γ polypeptide has at least 10% of the activity (e.g., binding to its receptor) of the corresponding wild type IL-36γ polypeptide. In a particular embodiment, the IL-36γ polypeptide comprises an amino acid sequence at least 50%, at least 60%, at least 70%, at least 80%, at least 85%, at least 90%, at least 95%, at least 98%, at least 99%, or 100% identical to SEQ ID NO: 27 and has at least 10% of the activity (e.g., binding to its receptor) of the corresponding wild type IL-36γ polypeptide. In another particular embodiment, the IL-36γ polypeptide consists essentially of SEQ ID NO: 27 and has at least 10% of the activity (e.g., binding to its receptor) of the corresponding wild type IL-36γ polypeptide.

In other embodiments, the IL-36γ polypeptide encoded by a mRNA of the disclosure comprises an amino acid sequence of SEQ ID NO: 27 or shown in SEQ ID NO: 27 with one or more conservative substitutions, wherein the conservative substitutions do not significantly affect the binding activity of the IL-36γ polypeptide to its receptor, i.e., the IL-36γ polypeptide binds to the IL-36γ receptor after the substitutions.

In some embodiments, a nucleotide sequence (i.e., mRNA) encoding an IL-36γ polypeptide comprises a sequence at least 50%, at least 60%, at least 70%, at least 80%, at least 85%, at least 90%, at least 95%, at least 98%, at least 99%, or 100% identical to a IL-36γ polypeptide encoding nucleic acid sequence listed in Table 1. In a particular embodiment, the nucleotide sequence (i.e., mRNA) encoding an IL-36γ polypeptide comprises a sequence at least 50%, at least 60%, at least 70%, at least 80%, at least 85%, at least 90%, at least 95%, at least 98%, at least 99%, or 100% identical to NO: 36 or SEQ ID NO: 29. In another particular embodiment, the nucleotide sequence (i.e., mRNA) encoding an IL-36γ polypeptide consists essentially of SEQ ID NO: 28 OR SEQ ID NO: 29. It should be understood that the nucleotide sequence (i.e., mRNA, e.g., SEQ ID NO: 28) encoding an IL-23 polypeptide open reading frame (ORF) can be one element within a larger construct, e.g., further including a 5′ terminal cap, 5′UTR, 3′UTR, and/or polyA tail.

The sequence summary presents, e.g., precursor and mature sequences for IL-23, IL-36γ, and OX40L as well as constructs comprising IL-23 or IL-36γ. In the context of the present disclosure IL-23 mRNA or IL-23 polypeptide encompass both “precursor” and “mature” forms. Furthermore, a construct comprising a mRNA encoding IL-23, IL-36γ, and OX40L and further comprising components such 3′ UTR and 5′ UTR would be considered an IL-23, IL-36γ, and OX40L encoding mRNA. A person of skill in the art would understand that in addition to the native signal sequences and propeptide sequences implicitly disclosed in the sequence summary (sequences present in the precursor for and absent in the mature corresponding form) and the non-native signal peptide disclosed in the sequence summary (IgKV4 signal peptide), other signal sequences can be used. Accordingly, references to an IL-23, IL-36-gamma, and OX40L polypeptide or mRNA according to the sequence summary encompass variants in which an alternative signal peptide (or encoding sequence) known in the art has been attached to said IL-23, IL-36γ, and OX40L polypeptide (or mRNA). It is also understood that references to the sequences disclosed in the sequence summary through the application are equally applicable and encompass orthologs and functional variants (for example polymorphic variants) and isoforms of those sequences known in the art at the time the application was filed

mRNA Construct Components

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). An exemplary 5′ UTR for use in the constructs is shown in SEQ ID NO: 15. Another exemplary 5′ UTR for use in the constructs is shown in SEQ ID NO: 16. Another exemplary 5′ UTR for use in the constructs is shown in SEQ ID NO: 12. An exemplary 3′ UTR for use in the constructs is shown in SEQ ID NO: 17. An exemplary 3′ UTR comprising miR-122 and miR-142.3p binding sites for use in the constructs is shown in SEQ ID NO: 18. An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m⁷G(5′)ppp(5′)G, commonly written as m⁷GpppG. A cap species may also be an anti-reverse cap analog. A non-limiting list of possible cap species includes m⁷GpppG, m⁷Gpppm⁷G, m⁷3′dGpppG, m₂^{7,O3′}GpppG, m₂^{7,O3′}GppppG, m₂^{7,O2′}GppppG, m⁷Gpppm⁷G, m⁷3′dGpppG, m₂^{7,O3′}GpppG, m₂^{7,O3′}GppppG, and m₂^{7,O2′}GppppG.

An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA binding site.

### MicroRNA Binding Sites

In some embodiments, the OX40L, IL-23 and IL-36γ encoding mRNAs comprises one or more microRNA binding sites. microRNAs (or miRNA) are 19-25 nucleotides long noncoding RNAs that bind to the 3′UTR of nucleic acid molecules and down-regulate gene expression either by reducing nucleic acid molecule stability or by inhibiting translation.

By engineering microRNA target sequences into an mRNA, one can target the molecule for degradation or reduced translation, provided the microRNA in question is available. In some embodiments, the miRNA binding site (e.g., miR-122 binding site) binds to the corresponding mature miRNA that is part of an active RNA-induced silencing complex (RISC) containing Dicer. In some embodiments, binding of the miRNA binding site to the corresponding miRNA in RISC degrades the mRNA containing the miRNA binding site or prevents the mRNA from being translated.

Some microRNAs, e.g., miR-122, are abundant in normal tissue but are present in much lower levels in cancer or tumor tissue. Thus, engineering microRNA target sequences (i.e., microRNA binding site) into the OX40L, IL-23 and IL-36γ encoding mRNAs (e.g., in a 3′UTR like region or other region) can effectively target the molecule for degradation or reduced translation in normal tissue (where the microRNA is abundant) while providing high levels of translation in the cancer or tumor tissue (where the microRNA is present in much lower levels). This provides a tumor-targeting approach for the methods and compositions of the disclosure.

In some embodiments, the microRNA binding site (e.g., miR-122 binding site) is fully complementary to miRNA (e.g., miR-122), thereby degrading the mRNA fused to the miRNA binding site. In other embodiments, the miRNA binding site is not fully complementary to the corresponding miRNA. In certain embodiments, the miRNA binding site (e.g., miR-122 binding site) is the same length as the corresponding miRNA (e.g., miR-122). The miRNA binding sites that are shorter than the corresponding miRNAs are still capable of degrading the mRNA incorporating one or more of the miRNA binding sites or preventing the mRNA from translation.

In some embodiments, the microRNA binding site (e.g., miR-122 binding site) has sufficient complementarity to miRNA (e.g., miR-122) so that a RISC complex comprising the miRNA (e.g., miR-122) cleaves the mRNA comprising the microRNA binding site. In other embodiments, the microRNA binding site (e.g., miR-122 binding site) has imperfect complementarity so that a RISC complex comprising the miRNA (e.g., miR-122) induces instability in the mRNA comprising the microRNA binding site. In another embodiment, the microRNA binding site (e.g., miR-122 binding site) has imperfect complementarity so that a RISC complex comprising the miRNA (e.g., miR-122) represses transcription of the mRNA comprising the microRNA binding site.

In some embodiments, the OX40L, IL-23 or IL-36γ encoding mRNAs comprise at least one miR-122 binding site, at least two miR-122 binding sites, at least three miR-122 binding sites, at least four miR-122 binding sites, or at least five miR-122 binding sites. In some embodiments, the miRNA binding site binds miR-122 or is complementary to miR-122. In some embodiments, the miRNA binding site binds to miR-122-3p or miR-122-5p. In some embodiments, the miRNA binding site comprises a nucleotide sequence at least 80%, at least 85%, at least 90%, at least 95%, or 100% identical to SEQ ID NO: 14, wherein the miRNA binding site binds to miR-122. In another particular aspect, the miRNA binding site comprises a nucleotide sequence at least 80%, at least 85%, at least 90%, at least 95%, or 100% identical to SEQ ID NO. 20, wherein the miRNA binding site binds to miR-122.

In some embodiments, a miRNA binding site (e.g., miR-122 binding site) is inserted in the mRNA in any position (e.g., 3′ UTR); the insertion site in the mRNA can be anywhere in the mRNA as long as the insertion of the miRNA binding site in the mRNA does not interfere with the translation of the functional OX40L, IL-23 and IL-36γ polypeptides in the absence of the corresponding miRNA (e.g., miR122); and in the presence of the miRNA (e.g., miR122), the insertion of the miRNA binding site in the mRNA and the binding of the miRNA binding site to the corresponding miRNA are capable of degrading the mRNA or preventing the translation of the mRNA. In one embodiment, a miRNA binding site is inserted in a 3′UTR of the mRNA.

In certain embodiments, a miRNA binding site is inserted in at least about 30 nucleotides downstream from the stop codons of the OX40L, IL-23 and/or IL-36γ encoding mRNA. In other embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codons of the OX40L, IL-23 and/or IL-36γ encoding mRNA. In other embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codons of the OX40L, IL-23 and/or IL-36γ encoding mRNAs.

Modified mRNAs

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is pseudouridine (ψ), N1-methylpseudouridine (m¹ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified cytosine. Exemplary nucleobases and nucleosides having a modified cytosine include N4-acetyl-cytidine (ac⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, 2-thio-cytidine (s²C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified adenine. Exemplary nucleobases and nucleosides having a modified adenine include 7-deaza-adenine, 1-methyl-adenosine (m¹A), 2-methyl-adenine (m²A), N6-methyl-adenosine (m⁶A). In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified guanine. Exemplary nucleobases and nucleosides having a modified guanine include inosine (I), 1-methyl-inosine (m¹I), wyosine (imG), methylwyosine (mimG), 7-deaza-guanosine, 7-cyano-7-deaza-guanosine (preQ₀), 7-aminomethyl-7-deaza-guanosine (preQ₁), 7-methyl-guanosine (m⁷G), 1-methyl-guanosine (m¹G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is 1-methyl-pseudouridine (m¹ψ), 5-methoxy-uridine (mo⁵U), 5-methyl-cytidine (m⁵C), pseudouridine (ψ), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases).

In some embodiments, the mRNA comprises pseudouridine (ψ). In some embodiments, the mRNA comprises pseudouridine (ψ) and 5-methyl-cytidine (m⁵C). In some embodiments, the mRNA comprises 1-methyl-pseudouridine (m¹ψ). In some embodiments, the mRNA comprises 1-methyl-pseudouridine (m¹ψ) and 5-methyl-cytidine (m⁵C). In some embodiments, the mRNA comprises 2-thiouridine (s²U). In some embodiments, the mRNA comprises 2-thiouridine and 5-methyl-cytidine (m⁵C). In some embodiments, the mRNA comprises 5-methoxy-uridine (mo⁵U). In some embodiments, the mRNA comprises 5-methoxy-uridine (mo⁵U) and 5-methyl-cytidine (m⁵C). In some embodiments, the mRNA comprises 2′-O-methyl uridine. In some embodiments, the mRNA comprises 2′-O-methyl uridine and 5-methyl-cytidine (m⁵C). In some embodiments, the mRNA comprises N6-methyl-adenosine (m⁶A). In some embodiments, the mRNA comprises N6-methyl-adenosine (m⁶A) and 5-methyl-cytidine (m⁵C).

In certain embodiments, an mRNA of the disclosure is uniformly modified (i.e., fully modified, modified through-out the entire sequence) for a particular modification. In some embodiments, an mRNA of the disclosure is modified wherein at least 95%, at least 96%, at least 97%, at least 98%, at least 99% or 100% of a specified nucleotide or nucleobase is modified. For example, an mRNA can be uniformly modified with 5-methyl-cytidine (m⁵C), meaning that all cytosine residues in the mRNA sequence are replaced with 5-methyl-cytidine (m⁵C). Similarly, mRNAs of the disclosure can be uniformly modified for any type of nucleoside residue present in the sequence by replacement with a modified residue such as those set forth above. In some embodiments, an mRNA of the disclosure is uniformly modified with 1-methyl pseudouridine (m¹ψ), meaning that all uridine residues in the mRNA sequence are replaced with 1-methyl pseudouridine (m¹ψ). In some embodiments, at least 95%, at least 96%, at least 97%, at least 98%, at least 99% or 100% of uridines are 1-methyl pseudouridine (m¹ψ).

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

Examples of nucleoside modifications and combinations thereof that may be present in mmRNAs of the present disclosure include, but are not limited to, those described in PCT Patent Application Publications: WO2012045075, WO2014081507, WO2014093924, WO2014164253, and WO2014159813.

The mRNAs of the disclosure can include a combination of modifications to the sugar, the nucleobase, and/or the internucleoside linkage. These combinations can include any one or more modifications described herein.

In certain embodiments, the modified nucleosides may be partially or completely substituted for the natural nucleotides of the mRNAs of the disclosure. As a non-limiting example, the natural nucleotide uridine may be substituted with a modified nucleoside described herein. In another non-limiting example, the natural nucleoside uridine may be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9% of the natural uridines) with at least one of the modified nucleoside disclosed herein.

The mRNAs of the present disclosure, or regions thereof, may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the mRNA. Codon optimization tools, algorithms and services are known in the art; non-limiting examples include services from GeneArt (Life Technologies), DNA2.0 (Menlo Park, Calif.) and/or proprietary methods. In one embodiment, the mRNA sequence is optimized using optimization algorithms, e.g., to optimize expression in mammalian cells or enhance mRNA stability.

In certain embodiments, the present disclosure includes mRNAs having at least 80%, at least 85%, at least 90%, at least 95%, at least 98%, or at least 99% sequence identity to any of the mRNA sequences described herein.

mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In one embodiment, mRNAs are made using IVT enzymatic synthesis methods. Methods of making mRNAs by IVT are known in the art and are described in International Application PCT/US2013/30062, the contents of which are incorporated herein by reference in their entirety. Accordingly, the present disclosure also includes mRNAs, e.g., DNA, constructs and vectors that may be used to in vitro transcribe an mRNA described herein.

Non-natural modified nucleobases may be introduced into mRNAs, e.g., mRNA, during synthesis or post-synthesis. In certain embodiments, modifications may be on internucleoside linkages, purine or pyrimidine bases, or sugar. In particular embodiments, the modification may be introduced at the terminal of a mRNA chain or anywhere else in the mRNA chain; with chemical synthesis or with a polymerase enzyme. Examples of modified nucleic acids and their synthesis are disclosed in PCT application No. PCT/US2012/058519. Synthesis of modified mRNAs is also described in Verma and Eckstein, Annual Review of Biochemistry, vol. 76, 99-134 (1998). Either enzymatic or chemical ligation methods may be used to conjugate mRNAs or their regions with different functional moieties, such as targeting or delivery agents, fluorescent labels, liquids, nanoparticles, etc. Conjugates of mRNAs and modified mRNAs are reviewed in Goodchild, Bioconjugate Chemistry, vol. 1(3), 165-187 (1990).

### Delivery Agents

Lipid Compound

The present disclosure provides pharmaceutical compositions with advantageous properties. The lipid compositions described herein may be advantageously used in lipid nanoparticle compositions for the delivery of therapeutic and/or prophylactic agents, e.g., mRNAs, to mammalian cells or organs. For example, the lipids described herein have little or no immunogenicity. For example, the lipid compounds disclosed herein have a lower immunogenicity as compared to a reference lipid (e.g., MC3, KC2, or DLinDMA). For example, a formulation comprising a lipid disclosed herein and a therapeutic or prophylactic agent, e.g., mRNA, has an increased therapeutic index as compared to a corresponding formulation which comprises a reference lipid (e.g., MC3, KC2, or DLinDMA) and the same therapeutic or prophylactic agent.

In certain embodiments, the present application provides pharmaceutical compositions comprising:

(a) mRNAs comprising a nucleotide sequence encoding OX40L, IL-23 and/or IL-36γ; and

(b) a delivery agent.

### Lipid Nanoparticle Formulations

In some embodiments, nucleic acids of the disclosure are formulated as lipid nanoparticle (LNP) compositions. Lipid nanoparticles typically comprise amino lipid, phospholipid, structural lipid and PEG lipid components along with the nucleic acid cargo of interest. The lipid nanoparticles of the invention can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575; PCT/US2016/069491; PCT/US2016/069493; and PCT/US2014/66242, all of which are incorporated by reference herein in their entirety.

In some embodiments, the lipid nanoparticle comprises a molar ratio of 20-60% amino lipid relative to the other lipid components. For example, the lipid nanoparticle may comprise a molar ratio of 20-50%, 20-40%, 20-30%, 30-60%, 30-50%, 30-40%, 40-60%, 40-50%, or 50-60% amino lipid. In some embodiments, the lipid nanoparticle comprises a molar ratio of 20%, 30%, 40%, 50, or 60% amino lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises a molar ratio of 20-60% amino lipid, 5-25% phospholipid, 25-55% structural lipid, and 0.5-15% PEG lipid.

In some embodiments, the lipid nanoparticle comprises a molar ratio of 20-60% amino lipid, 5-30% phospholipid, 10-55% structural lipid, and 0.5-15% PEG lipid.

### Amino Lipids

In some aspects, the amino lipids of the present disclosure may be one or more of compounds of Formula (I):

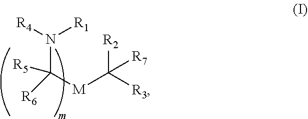

or their N-oxides, or salts or isomers thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of hydrogen, a C₃₋₆
carbocycle, —(CH₂)ₙQ, —(CH₂)ₙCHQR,
—CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈,
—N(R)S(O)₂R₈, —O(CH₂)ₙOR, —N(R)C(═NR₉)N(R)₂, —N(R)C(═CHR₉)N(R)₂, —OC(O)N(R)₂,
—N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected
from —C(O)O—, —OC(O)—, —OC(O)-M″-C(O)O—, —C(O)N(R′)—,
—N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13; and wherein when R₄
is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In certain embodiments, a subset of compounds of Formula (I) includes those of Formula (IA):

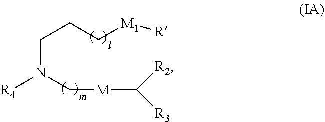

or its N-oxide, or a salt or isomer thereof, wherein l is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (IB):

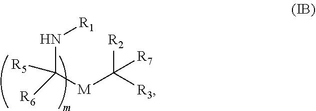

or its N-oxide, or a salt or isomer thereof in which all variables are as defined herein. For example, m is selected from 5, 6, 7, 8, and 9; R₄ is hydrogen, unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (II):

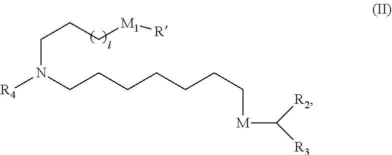

or its N-oxide, or a salt or isomer thereof, wherein l is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In one embodiment, the compounds of Formula (I) are of Formula (IIa),

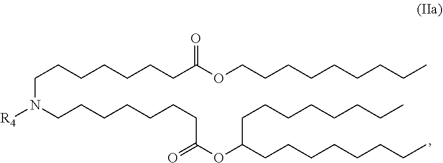

or their N-oxides, or salts or isomers thereof, wherein R₄ is as described herein.

In another embodiment, the compounds of Formula (I) are of Formula (IIb),

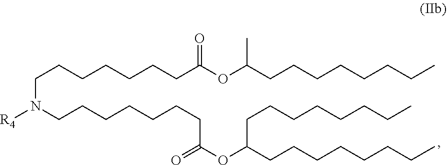

or their N-oxides, or salts or isomers thereof, wherein R₄ is as described herein.

In another embodiment, the compounds of Formula (I) are of Formula (IIc) or (IIe);

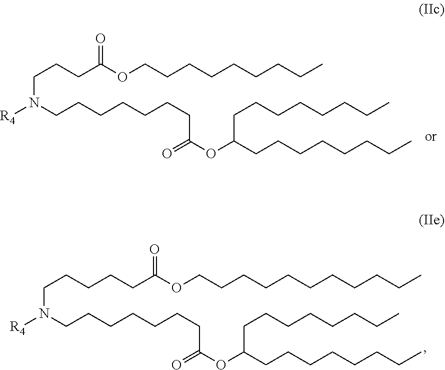

or their N-oxides, or salts or isomers thereof, wherein R₄ is as described herein.
In another embodiment, the compounds of Formula (I) are of Formula (IIf):

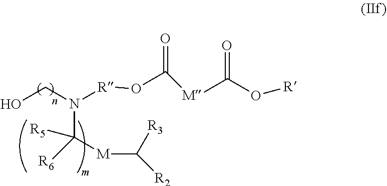

or their N-oxides, or salts or isomers thereof, wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (I) are of Formula (IId),

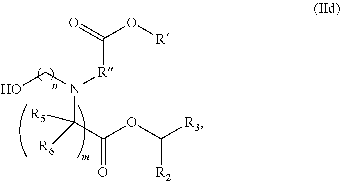

or their N-oxides, or salts or isomers thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In a further embodiment, the compounds of Formula (I) are of Formula (IIg),

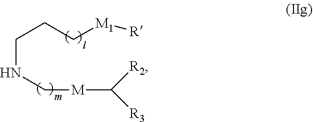

or their N-oxides, or salts or isomers thereof, wherein l is selected from 1, 2, 3, 4, and 5; m is selected from 5, 6, 7, 8, and 9; M₁ is a bond or M′; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

In some embodiments, the amino lipid is

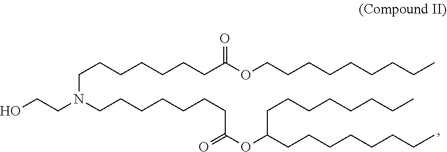 [SMILES: CCCCCCCCCOC(=O)CCCCCCCN(CCO)CCCCCCCC(=O)OC(CCCCCCCC)CCCCCCCC]

or a salt thereof.

In some embodiments, the amino lipid is

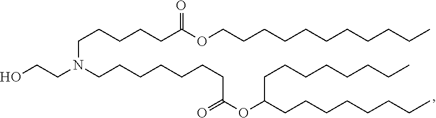 [SMILES: CCCCCCCCCCCOC(=O)CCCCCN(CCO)CCCCCCCC(=O)OC(CCCCCCCC)CCCCCCCC]

or a salt thereof.

The central amine moiety of a lipid according to Formula (I), (IA), (IB), (II), (IIa), (IIb), (IIc), (IId), (IIe), (IIf), or (IIg) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

In some aspects, the amino lipids of the present disclosure may be one or more of compounds of formula (III),

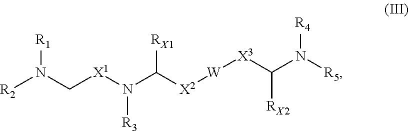

or salts or isomers thereof, wherein

W is

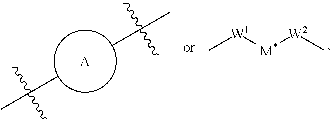

ring A is

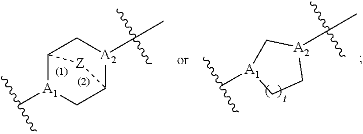

t is 1 or 2;

A₁ and A₂ are each independently selected from CH or N;

Z is CH₂ or absent wherein when Z is CH₂, the dashed lines (1) and (2) each represent a single bond; and when Z is absent, the dashed lines (1) and (2) are both absent;

R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;

R_{X1} and R_{X2} are each independently H or C₁₋₃ alkyl;

each M is independently selected from the group consisting

of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;

M* is C₁-C₆ alkyl,

W¹ and W² are each independently selected from the group consisting of —O— and —N(R₆)—;

each R₆ is independently selected from the group consisting of H and C₁₋₅ alkyl;

X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;

each Y is independently a C₃₋₆ carbocycle;

each R* is independently selected from the group consisting of C₁₋₁₂ alkyl and C₂₋₁₂ alkenyl;

each R is independently selected from the group consisting of C₁₋₃ alkyl and a C₃₋₆ carbocycle;

each R′ is independently selected from the group consisting of C₁₋₁₂ alkyl, C₂₋₁₂ alkenyl, and H;

each R″ is independently selected from the group consisting of C₃₋₁₂ alkyl, C₃₋₁₂ alkenyl and —R*MR′; and

n is an integer from 1-6;

wherein when ring A is

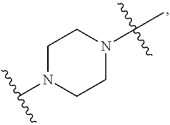 [SMILES: *N1CCN(*)CC1]

then

i) at least one of X¹, X², and X³ is not —CH₂—; and/or

ii) at least one of R₁, R₂, R₃, R₄, and R₅ is —R″MR′.

In some embodiments, the compound is of any of formulae (IIIa1)-(IIIa8):

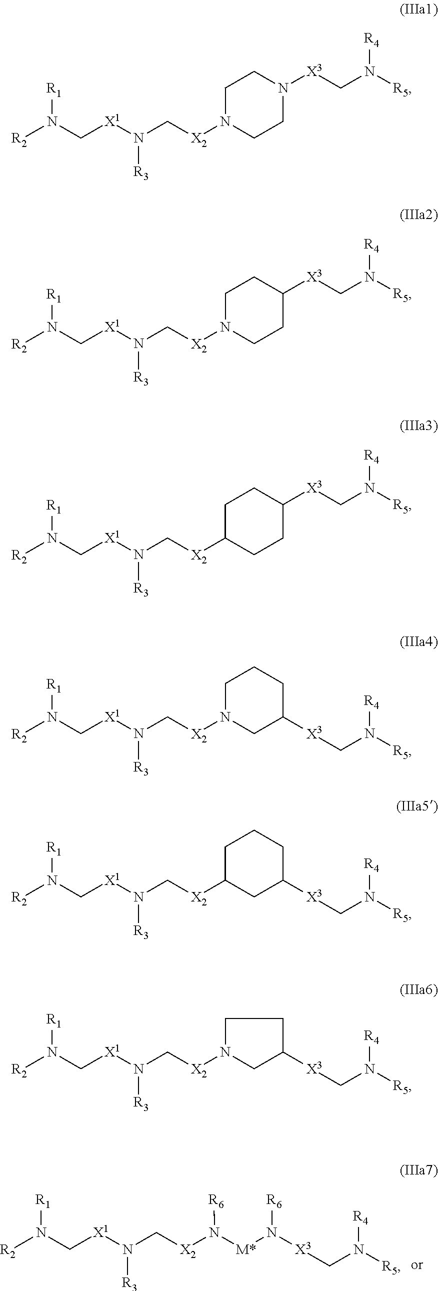

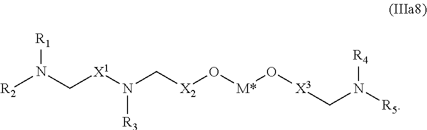

In some embodiments, the amino lipid is

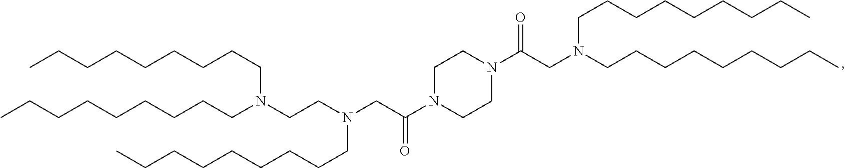 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (III), (IIIa1), (IIIa2), (IIIa3), (IIIa4), (IIIa5), (IIIa6), (IIIa7), or (IIIa8) may be protonated at a physiological pH. Thus, a lipid may have a positive or partial positive charge at physiological pH.

### Phospholipids

The lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidyl glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present invention is an analog or variant of DSPC. In certain embodiments, a phospholipid useful or potentially useful in the present invention is a compound of Formula (IV):

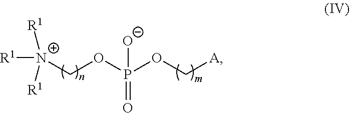

or a salt thereof, wherein:

each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;

n is 1, 2, 3, 4, 5, 6, 7, 8, 9, or 10;

m is 0, 1, 2, 3, 4, 5, 6, 7, 8, 9, or 10;

A is of the formula:

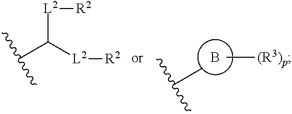

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}) NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2;

provided that the compound is not of the formula:

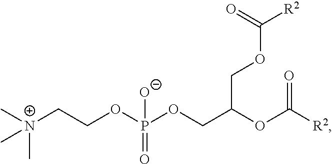

wherein each instance of R² is independently unsubstituted alkyl, unsubstituted alkenyl, or unsubstituted alkynyl.

In some embodiments, the phospholipids may be one or more of the phospholipids described in U.S. Application No. 62/520,530.

### Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

### Polyethylene Glycol (PEG)-Lipids

The lipid composition of a pharmaceutical composition disclosed herein can comprise one or more polyethylene glycol (PEG) lipids.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-distearyl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example a mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

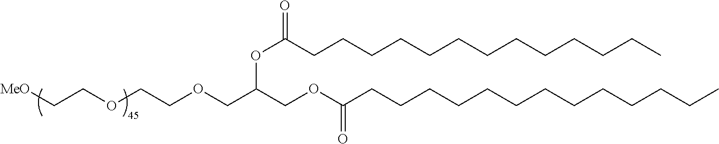

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (V). Provided herein are compounds of Formula (V):

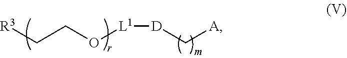

or salts thereof, wherein:

R³ is —OR^{O};

R^{O} is hydrogen, optionally substituted alkyl, or an oxygen protecting group;

r is an integer between 1 and 100, inclusive;

L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

D is a moiety obtained by click chemistry or a moiety cleavable under physiological conditions;

m is 0, 1, 2, 3, 4, 5, 6, 7, 8, 9, or 10;

A is of the formula:

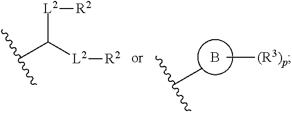

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2.

In certain embodiments, the compound of Formula (V) is a PEG-OH lipid (i.e., R³ is —OR^{O}, and R^{O} is hydrogen). In certain embodiments, the compound of Formula (V) is of Formula (V-OH):

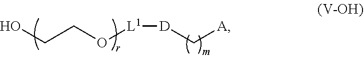

or a salt thereof.

In certain embodiments, a PEG lipid useful in the present invention is a PEGylated fatty acid. In certain embodiments, a PEG lipid useful in the present invention is a compound of Formula (VI). Provided herein are compounds of Formula (VI):

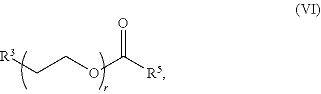

or a salts thereof, wherein:

R³ is —OR^{O};

R^{O} is hydrogen, optionally substituted alkyl or an oxygen protecting group;

r is an integer between 1 and 100, inclusive;

R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}) NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O; and

each instance of R^{N} is independently hydrogen, optionally substituted alkyl, or a nitrogen protecting group.

In certain embodiments, the compound of Formula (VI) is of Formula (VI-OH):

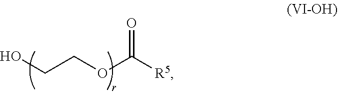

or a salt thereof. In some embodiments, r is 40-50.

In yet other embodiments the compound of Formula (VI) is:

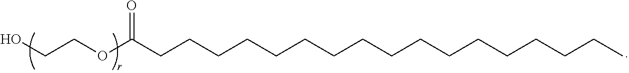

or a salt thereof.

In one embodiment, the compound of Formula (VI) is

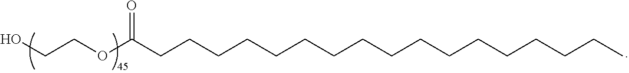

In some aspects, the lipid composition of the pharmaceutical compositions disclosed herein does not comprise a PEG-lipid.

In some embodiments, the PEG-lipids may be one or more of the PEG lipids described in U.S. application Ser. No. 15/674,872.

In some embodiments, a LNP of the invention comprises an amino lipid of any of Formula I, II or III, a phospholipid comprising DSPC, a structural lipid, and a PEG lipid comprising PEG-DMG.

In some embodiments, a LNP of the invention comprises an amino lipid of any of Formula I, II or III, a phospholipid comprising DSPC, a structural lipid, and a PEG lipid comprising a compound having Formula VI.

In some embodiments, a LNP of the invention comprises an amino lipid of Formula I, II or III, a phospholipid comprising a compound having Formula IV, a structural lipid, and the PEG lipid comprising a compound having Formula V or VI.

In some embodiments, a LNP of the invention comprises an amino lipid of Formula I, II or III, a phospholipid comprising a compound having Formula IV, a structural lipid, and the PEG lipid comprising a compound having Formula V or VI.

In some embodiments, a LNP of the invention comprises an amino lipid of Formula I, II or III, a phospholipid having Formula IV, a structural lipid, and a PEG lipid comprising a compound having Formula VI.

In some embodiments, a LNP of the invention comprises an N:P ratio of from about 2:1 to about 30:1.

In some embodiments, a LNP of the invention comprises an N:P ratio of about 6:1.

In some embodiments, a LNP of the invention comprises an N:P ratio of about 3:1, 4:1, or 5:1.

In some embodiments, a LNP of the invention comprises a wt/wt ratio of the amino lipid component to the RNA of from about 10:1 to about 100:1.

In some embodiments, a LNP of the invention comprises a wt/wt ratio of the amino lipid component to the RNA of about 20:1.

In some embodiments, a LNP of the invention comprises a wt/wt ratio of the amino lipid component to the RNA of about 10:1.

In some embodiments, a LNP of the invention has a mean diameter from about 30 nm to about 150 nm.

In some embodiments, a LNP of the invention has a mean diameter from about 60 nm to about 120 nm.

In some embodiments, a LNP of the disclosure comprises the mRNA therapeutic agent described herein in a concentration from about 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml. In some embodiments, a LNP of the disclosure comprises the mRNA therapeutic agent described herein in a concentration of about 2.0 mg/ml.

### Pharmaceutical Compositions

The present disclosure includes pharmaceutical compositions comprising OX40L, IL-23 and IL-36γ encoding mRNAs or a nanoparticle (e.g., a lipid nanoparticle) described herein, in combination with one or more pharmaceutically acceptable excipient, carrier or diluent. In particular embodiments, the mRNA is present in a nanoparticle, e.g., a lipid nanoparticle. In particular embodiments, the mRNA or nanoparticle is present in a pharmaceutical composition.

Pharmaceutical compositions may optionally include one or more additional active substances, for example, therapeutically and/or prophylactically active substances. Pharmaceutical compositions of the present disclosure may be sterile and/or pyrogen-free. General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference in its entirety). In particular embodiments, a pharmaceutical composition comprises an mRNA and a lipid nanoparticle, or complexes thereof.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may include between 0.1% and 100%, e.g., between 0.5% and 70%, between 1% and 30%, between 5% and 80%, or at least 80% (w/w) active ingredient.

The mRNAs of the disclosure can be formulated using one or more excipients to: (1) increase stability; (2) increase cell transfection; (3) permit the sustained or delayed release (e.g., from a depot formulation of the mRNA); (4) alter the biodistribution (e.g., target the mRNA to specific tissues or cell types); (5) increase the translation of a polypeptide encoded by the mRNA in vivo; and/or (6) alter the release profile of a polypeptide encoded by the mRNA in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles (e.g., liposomes and micelles), polymers, lipoplexes, core-shell nanoparticles, peptides, proteins, carbohydrates, cells transfected with mRNAs (e.g., for transplantation into a subject), hyaluronidase, nanoparticle mimics and combinations thereof. Accordingly, the formulations of the disclosure can include one or more excipients, each in an amount that together increases the stability of the mRNA, increases cell transfection by the mRNA, increases the expression of a polypeptide encoded by the mRNA, and/or alters the release profile of an mRNA-encoded polypeptide. Further, the mRNAs of the present disclosure may be formulated using self-assembled nucleic acid nanoparticles.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition. Excipients may include, for example: antiadherents, antioxidants, binders, coatings, compression aids, disintegrants, dyes (colors), emollients, emulsifiers, fillers (diluents), film formers or coatings, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

In some embodiments, the formulations described herein may include at least one pharmaceutically acceptable salt. Examples of pharmaceutically acceptable salts that may be included in a formulation of the disclosure include, but are not limited to, acid addition salts, alkali or alkaline earth metal salts, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

In some embodiments, the formulations described herein may contain at least one type of mRNA. As a non-limiting example, the formulations may contain 1, 2, 3, 4, 5 or more than 5 mRNAs described herein. In some embodiments, the formulations described herein may contain at least one mRNA encoding a polypeptide and at least one nucleic acid sequence such as, but not limited to, an siRNA, an shRNA, a snoRNA, and an miRNA.

Liquid dosage forms for e.g., parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and/or suspending agents. In certain embodiments for parenteral administration, compositions are mixed with solubilizing agents such as CREMAPHOR®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In some embodiments, pharmaceutical compositions including at least one mRNA described herein are administered to mammals (e.g., humans). Although the descriptions of pharmaceutical compositions provided herein are principally directed to pharmaceutical compositions which are suitable for administration to humans, it will be understood by the skilled artisan that such compositions are generally suitable for administration to any other animal, e.g., to a non-human mammal. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys. In particular embodiments, a subject is provided with two or more mRNAs described herein. In particular embodiments, the first and second mRNAs are provided to the subject at the same time or at different times, e.g., sequentially. In particular embodiments, the first and second mRNAs are provided to the subject in the same pharmaceutical composition or formulation, e.g., to facilitate uptake of both mRNAs by the same cells.

The present disclosure also includes kits comprising a container comprising a mRNA encoding a polypeptide that enhances an immune response. In another embodiment, the kit comprises a container comprising a mRNA encoding a polypeptide that enhances an immune response, as well as one or more additional mRNAs encoding one or more antigens or interest. In other embodiments, the kit comprises a first container comprising the mRNA encoding a polypeptide that enhances an immune response and a second container comprising one or more mRNAs encoding one or more antigens of interest. In particular embodiments, the mRNAs for enhancing an immune response and the mRNA(s) encoding an antigen(s) are present in the same or different nanoparticles and/or pharmaceutical compositions. In particular embodiments, the mRNAs are lyophilized, dried, or freeze-dried.

### Kits

In some embodiments, the disclosure provides a kit comprising OX40L, IL-23 and IL-36γ encoding mRNAs, or composition (e.g. lipid nanoparticle) comprising OX40L, IL-23 and IL-36γ encoding mRNAs, as described herein. In some embodiments, a kit comprises a container comprising a pharmaceutical composition comprising a lipid nanoparticle comprising mRNAs encoding human OX40L, IL-23 and IL-36γ polypeptides; and a pharmaceutically acceptable carrier, wherein the pharmaceutical composition comprises 2 mg/ml of the mRNA, and a package insert comprising instructions for administration of the mRNA by intratumoral injection to treat or delay progression of solid tumor malignancy or lymphoma in a human patient.

In some embodiments, a kit comprises a container comprising a pharmaceutical composition comprising a lipid nanoparticle comprising mRNAs encoding human OX40L, IL-23 and IL-36γ polypeptides; and a pharmaceutically acceptable carrier, wherein the pharmaceutical composition comprises 2 mg/ml of the mRNA, and a package insert comprising instructions for administration of the mRNA by intratumoral injection and instruction for use in combination with a second composition comprising a PD-1 antagonist, a PD-L1 antagonist or a CTLA-4 antagonist, for use in treating or delaying progression of solid tumor malignancy or lymphoma in a human patient.

In some embodiments, a kit comprises a container comprising a lipid nanoparticle encapsulating the mRNA described herein, and an optional pharmaceutically acceptable carrier, or a pharmaceutical composition, and a package insert comprising instructions for administration of the lipid nanoparticle or pharmaceutical composition. In some embodiments, a kit comprises a container comprising a lipid nanoparticle encapsulating the mRNA described herein, and an optional pharmaceutically acceptable carrier, or a pharmaceutical composition, and a package insert comprising instructions for administration of the lipid nanoparticle or pharmaceutical composition for treating or delaying progression of an solid tumor malignancy or lymphoma in an individual. In some aspects, the package insert further comprises instructions for administration of the lipid nanoparticle or pharmaceutical composition in combination with a composition comprising a checkpoint inhibitor polypeptide and an optional pharmaceutically acceptable carrier for treating or delaying progression of an solid tumor malignancy or lymphoma in an individual.

In some embodiments, a kit comprises a medicament comprising a lipid nanoparticle encapsulating the OX40L, IL-23 and IL-36γ encoding mRNAs described herein, and an optional pharmaceutically acceptable carrier, or a pharmaceutical composition, and a package insert comprising instructions for administration of the medicament alone or in combination with a composition comprising a checkpoint inhibitor polypeptide and an optional pharmaceutically acceptable carrier. In some embodiments, a kit comprises a medicament comprising a lipid nanoparticle encapsulating the OX40L, IL-23 and IL-36γ encoding mRNAs described herein, and an optional pharmaceutically acceptable carrier, or a pharmaceutical composition, and a package insert comprising instructions for administration of the medicament alone or in combination with a composition comprising a checkpoint inhibitor polypeptide and an optional pharmaceutically acceptable carrier for treating or delaying progression of solid tumor malignancy or lymphoma in an individual. In some aspects, the kit further comprises a package insert comprising instructions for administration of the first medicament prior to, current with, or subsequent to administration of the second medicament for treating or delaying progression of solid tumor malignancy or lymphoma in an individual.

### Other Embodiments

The disclosure relates to the following embodiments. Throughout this section, the term embodiment is abbreviated as ‘E’ followed by an ordinal. For example, E1 is equivalent to Embodiment 1.

E1. A method for treating advanced or metastatic solid tumor malignancy or lymphoma in a human patient by inducing or enhancing an anti-tumor immune response, comprising administering to the patient by intratumoral injection an effective amount of a lipid nanoparticle (LNP) encapsulated messenger RNA (mRNA) therapeutic agent comprising three mRNA drug substances:

(i) a first mRNA comprising an open reading frame (ORF) encoding a human OX40L polypeptide;

(ii) a second mRNA comprising an ORF encoding a human IL-23 polypeptide; and

(iii) a third mRNA comprising an ORF encoding a human IL-36γ polypeptide,

wherein the patient is administered a dose of 0.10-10.0 mg of the mRNA therapeutic agent in a dosing regimen from 7 to 28 days, thereby treating the advanced or metastatic solid tumor malignancy or lymphoma in the patient by inducing or enhancing an anti-tumor immune response.

E2. The method of embodiment 1, wherein the patient is administered a dose of the mRNA therapeutic agent selected from: 0.25-8.0 mg; 0.25-4.0 mg; 0.25-2.0 mg; 0.25-1.0 mg; 0.25-5 mg; 0.5-8.0 mg; 0.5-4.0 mg; 0.5-2.0 mg; 0.5-1.0 mg; 1.0-8.0 mg; 1.0-4.0 mg; 1.0-2.0 mg; 2.0-8.0 mg; 2.0-4.0 mg; and 4.0-8.0 mg.
E3. The method of embodiment 1, wherein the patient is administered a dose of 0.10 mg, 0.25 mg, 0.50 mg, 1.0 mg, 2.0 mg, 4.0 mg, 8.0 mg or 10.0 mg of the mRNA therapeutic agent.
E4. The method of any one of the preceding embodiments, wherein the mRNA therapeutic agent is administered to the patient in a dosing regimen selected from 7 to 21 days, 7 to 14 days, 28 days, 21 days, 14 days, and 7 days.
E5. The method of any one of embodiments 1-4, wherein the patient is administered a dose of the mRNA therapeutic agent every 2 weeks.
E6. The method of any one of embodiments 1-4, wherein the patient is administered a dose of the mRNA therapeutic agent every 3 weeks.
E7. The method of any one of embodiments 1-4, wherein the patient is administered a dose of the mRNA therapeutic agent every 4 weeks.
E8. The method of any one of embodiments 1-4, wherein the patient is administered a dose of the mRNA therapeutic in a first dosing cycle of every 2 weeks, and at least one subsequent dosing cycle of every 4 weeks.
E9. The method of any one of embodiments 1-4, wherein the patient is administered a dose of the mRNA therapeutic in a first dosing cycle of every 2 weeks, and at least three subsequent dosing cycles of every 4 weeks.
E10. A method for treating advanced or metastatic solid tumor malignancy or lymphoma in a human patient by inducing or enhancing an anti-tumor immune response, comprising administering to the patient by intratumoral injection an effective amount of an LNP encapsulated mRNA therapeutic agent comprising three mRNA drug substances:

(i) a first mRNA comprising an ORF encoding a human OX40L polypeptide;

(ii) a second mRNA comprising an ORF encoding a human IL-23 polypeptide; and

(iii) a third mRNA comprising an ORF encoding a human IL-36γ polypeptide,

wherein the patient is administered a dose of the mRNA therapeutic agent selected from 0.25 mg, 0.5 mg, 1 mg, 2 mg, 4 mg, and 8 mg, thereby treating the advanced or metastatic solid tumor malignancy or lymphoma in the patient by inducing or enhancing an anti-tumor immune response.

E11. The method of embodiment 10, wherein the patient is administered a dose of the mRNA therapeutic agent every 2 weeks.
E12. The method of embodiment 10, wherein the patient is administered a dose of the mRNA therapeutic agent every 3 weeks.
E13. The method of embodiment 10, wherein the patient is administered a dose of the mRNA therapeutic agent every 4 weeks.
E14. The method of embodiment 10, wherein the patient is administered a dose of the mRNA therapeutic in a first dosing cycle of every 2 weeks, and at least one subsequent dosing cycle of every 4 weeks.
E15. The method of embodiment 10, wherein the patient is administered a dose of the mRNA therapeutic in a first dosing cycle of every 2 weeks, and at least three subsequent dosing cycles of every 4 weeks.
E16. The method of any one of the preceding embodiments, further comprising administering an effective amount of a PD-1 antagonist, a PD-L1 antagonist or a CTLA-4 antagonist.
E17. The method of embodiment 16, wherein the PD-1 antagonist is an antibody or antigen binding portion thereof that specifically binds to PD-1.
E18. The method of embodiment 16, wherein the PD-L1 antagonist is an antibody or antigen binding portion thereof that specifically binds to PD-L1.
E19. The method of embodiment 16, wherein the CTLA-4 antagonist is an antibody or antigen binding portion thereof that specifically binds to CTLA-4.
E20. The method of embodiment 17, wherein the PD-1 antagonist is selected from the group consisting of nivolumab, pembrolizumab, and pidilizumab.
E21. The method of embodiment 18, wherein the PD-L1 antagonist is selected from the group consisting of durvalumab, avelumab, and atezolizumab.
E22. The method of embodiment 21, wherein the PD-L1 antagonist is durvalumab.
E23. The method of embodiment 19, wherein the CTLA-4 antagonist is selected from the group consisting of ipilimumab and tremelimumab.
E24. The method of embodiment 23, wherein the CTLA-4 antagonist is tremelimumab.
E25. The method of any one of embodiments 16-24, wherein the patient is administered a dose of the PD-1 antagonist, the PD-L1 antagonist or CTLA-4 antagonist every 4 weeks.
E26. The method of any one of embodiments 16-25, wherein the patient is administered a dose of the mRNA therapeutic agent prior to administration of the PD-1 antagonist, PD-L1 antagonist or CTLA-4 antagonist.
E27. The method of any one of the preceding embodiments, wherein the advanced or metastatic solid tumor malignancy in the patient is selected from triple negative breast cancer, head and neck squamous cell carcinoma, and melanoma and the lymphoma is Non-Hodgkin lymphoma.
E28. A method for treating triple negative breast cancer (TNBC) in a human patient, comprising administering to the patient:

(a) an effective amount of an LNP encapsulated mRNA therapeutic agent comprising three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide; and

(b) an effective amount of PD-L1 antagonist selected from the group consisting of durvalumab, avelumab, and atezolizumab,

thereby treating the TNBC in the patient.

E29. A method for treating head and neck squamous cell carcinoma (HNSCC) in a human patient, comprising administering to the patient:

(a) an effective amount of an LNP encapsulated mRNA therapeutic agent comprising three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide; and

(b) an effective amount of PD-L1 antagonist selected from the group consisting of durvalumab, avelumab, and atezolizumab,

thereby treating the HNSCC in the patient.

E30. A method for treating Non-Hodgkin lymphoma (NHL) in a human patient, comprising administering to the patient:

(a) an effective amount of an LNP encapsulated mRNA therapeutic agent comprising three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide; and

(b) an effective amount of PD-L1 antagonist selected from the group consisting of durvalumab, avelumab, and atezolizumab,

thereby treating the NHL in the patient.

E31. A method for treating melanoma in a human patient, comprising administering to the patient:

(a) an effective amount of an LNP encapsulated mRNA therapeutic agent comprising three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide; and

(b) an effective amount of CTLA-4 antagonist selected from the group consisting of selected from the group consisting of ipilimumab and tremelimumab,

thereby treating the melanoma in the patient.

E32. A method for treating bladder cancer in a human patient, comprising administering to the patient:

(a) an effective amount of an LNP encapsulated mRNA therapeutic agent comprising three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide; and

(b) an effective amount of PD-L1 antagonist selected from the group consisting of durvalumab, avelumab, and atezolizumab,

thereby treating the bladder cancer in the patient.

E33. The method of embodiment 32, wherein the bladder cancer is a urothelial cancer.
E34. The method of embodiment 33, wherein the patient has received or is receiving platinum-based chemotherapy.
E35. The method of embodiment 33, wherein the patient is ineligible for platinum-based chemotherapy.
E36. The method of embodiment 32, wherein the bladder cancer is a squamous-cell bladder cancer.
E37. The method of embodiment 36, wherein the squamous-cell bladder cancer is PD-L1 negative or expresses low levels of PD-L1.
E38. The method of any one of embodiments 28-37, wherein the mRNA therapeutic agent is administered to the patient by intratumoral injection.
E39. The method of any one of embodiments 28-38, wherein the PD-L1 antagonist or CTLA-4 antagonist is administered to the patient by intravenous injection.
E40. The method of any one of embodiments 28-30 and 32-39, wherein the PD-L1 antagonist is durvalumab.
E41. The method of embodiment 40, wherein the patient is administered a dose of durvalumab of 1500 mg.
E42. The method of any one of embodiments 31 and 38-39, wherein the CTLA-4 antagonist is tremelimumab.
E43. The method of embodiment 42, wherein the patient is administered a dose of tremelimumab of 225 mg.
E44. The method of any one of embodiments 28-43, wherein the patient is administered a dose of the mRNA therapeutic agent selected from 0.25 mg, 0.5 mg, 1 mg, 2 mg, 4 mg, and 8 mg.
E45. The method of any one of embodiments 28-44, wherein the patient is administered a dose of the mRNA therapeutic agent every 4 weeks.
E46. The method of any one of embodiments 28-44, wherein the patient is administered a dose of the PD-L1 antagonist or CTLA-4 antagonist every 4 weeks.
E47. The method of any one of embodiments 28-46, wherein the mRNA therapeutic agent and the PD-L1 antagonist or the CTLA-4 antagonist are administered to the patient in a dosing regimen selected from 7 to 28 days, 7 to 21 days, 7 to 14 days, 28 days, 21 days, 14 days, and 7 days.
E48. The method of any one of embodiments 28-47, wherein the mRNA therapeutic agent and the PD-L1 antagonist or the CTLA-4 antagonist are administered to the patient in a dosing regimen of 28 days.
E49. The method of any one of embodiments 28-48, wherein the patient is administered a dose of the mRNA therapeutic agent prior to administration of the PD-L1 antagonist or the CTLA-4 antagonist.
E50. The method of any one of the preceding embodiments, wherein the human OX40L polypeptide comprises the amino acid sequence as set forth in SEQ ID NO: 1; the human IL-23 polypeptide comprises the amino acid sequence as set forth in SEQ ID NO: 24; and the human IL-36γ polypeptide comprises the amino acid sequence as set forth in SEQ ID NO: 27.
E51. The method of any one of the preceding embodiments, wherein

(i) the first mRNA encoding a human OX40L polypeptide comprises an ORF comprising a nucleotide sequence at least 80% identical to the nucleotide sequence set forth in SEQ ID NO: 4 or comprises the nucleotide sequence set forth in SEQ ID NO: 4;

(ii) the second mRNA encoding a human IL-23 polypeptide comprises an ORF comprising a nucleotide sequence at least 80% identical to the nucleotide sequence set forth in SEQ ID NO: 25 or comprises the nucleotide sequence set forth in SEQ ID NO: 25; and

(iii) the third mRNA encoding a human IL-36γ polypeptide comprises an ORF comprising a nucleotide sequence at least 80% identical to the nucleotide sequence set forth in SEQ ID NO: 28 or comprises the nucleotide sequence set forth in SEQ ID NO: 28.

E52. The method of any one of the preceding embodiments, wherein each of the first mRNA, second mRNA, and third mRNA comprise a 3′ untranslated region (UTR) comprising at least one microRNA-122 (miR-122) binding site.
E53. The method of embodiment 52, wherein the miR-122 binding site is a miR-122-3p binding site or a miR-122-5p binding site.
E54. The method of embodiment 53, wherein the miR-122-5p binding site comprises the nucleotide sequence set forth in SEQ ID NO: 20.
E55. The method of embodiment 52, wherein the 3′UTR comprises a nucleotide sequence at least 90% identical to the sequence set forth in SEQ ID NO: 17 or comprises the nucleotide sequence as set forth in SEQ ID NO: 17.
E56. The method of any one of the preceding embodiments, wherein each of the first, second, and third mRNAs comprise a 5′cap, a 5′ untranslated region (UTR), and a poly-A tail of about 100 nucleotides in length.
E57. The method of embodiment 56, wherein the 5′UTR comprises a nucleotide sequence at least 90% identical to the sequence set forth in SEQ ID NO: 16 or comprises the nucleotide sequence as set forth in SEQ ID NO: 16.
E58. The method of any one of embodiments 1-50, wherein

(i) the first mRNA encoding the human OX40L polypeptide comprises a nucleotide sequence at least 90% identical to the nucleotide sequence set forth in SEQ ID NO: 5 or comprises the nucleotide sequence set forth in SEQ ID NO: 5;

(ii) the second mRNA encoding a human IL-23 polypeptide comprises a nucleotide sequence at least 90% identical to the nucleotide sequence set forth in SEQ ID NO: 26 or comprises the nucleotide sequence set forth in SEQ ID NO: 26; and

(iii) the third mRNA encoding a human IL-36γ polypeptide comprises a nucleotide sequence at least 90% identical to the nucleotide sequence set forth in SEQ ID NO: 29 or comprises the nucleotide sequence set forth in SEQ ID NO: 29.

E59. The method of any one of the preceding embodiments, wherein the first, second, and third mRNAs are formulated in the lipid nanoparticle at a mass ratio of OX40L:IL-23:IL-36γ of 1:1:2.
E60. The method of any one of the preceding embodiments, wherein each of the first, second and third mRNAs is chemically modified.
E61. The method of embodiment 60, wherein each of the first, second, and third mRNAs is fully modified with chemically-modified uridines.
E62. The method of embodiment 61, wherein the chemically-modified uridines are N1-methylpseudouridines (m1ψ).
E63. The method of embodiment 60, wherein each of the first, second and third mRNAs is fully modified with 5-methylcytosine or is fully modified with N1-methylpseudouridines (m1ψ) and 5-methylcytosine.
E64. The method of any one of the preceding embodiments, wherein the LNP comprises a compound having the formula:

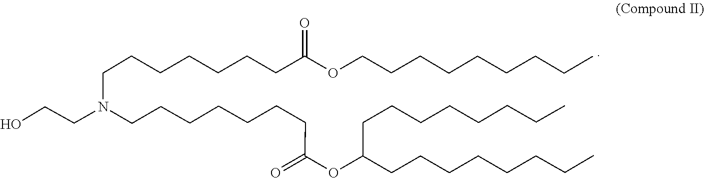 [SMILES: CCCCCCCCCOC(=O)CCCCCCCN(CCO)CCCCCCCC(=O)OC(CCCCCCCC)CCCCCCCC]

E65. The method of embodiment 64, wherein the LNP further comprises a phospholipid, a structural lipid, and a PEG lipid.
E66. The method of embodiment 65, wherein the LNP comprises a molar ratio of about 20-60% ionizable amino lipid, about 5-25% phospholipid, about 25-55% structural lipid, and about 0.5-1.5% PEG lipid.
E67. The method of embodiment 65, wherein the LNP comprises a molar ratio of about 50% ionizable amino lipid, about 10% phospholipid, about 38.5% structural lipid, and about 1.5% PEG lipid.
E68. The method of embodiment 65, wherein the LNP comprises a molar ratio of about 50% ionizable amino lipid, about 10% phospholipid, about 38.5% cholesterol, and about 1.5% PEG-DMG.
E69. The method of any one of the preceding embodiments, wherein the mRNA therapeutic agent is administered by a single injection.
E70. The method of any one of embodiments 1-69, wherein the mRNA therapeutic agent is administered by multiple injections into one or more different sites within the same tumor lesion or divided across several tumor lesions.
E71. The method of any one of the preceding embodiments, wherein the LNP is formulated in a pharmaceutically acceptable carrier.
E72. The method of embodiment 71, wherein the pharmaceutically acceptable carrier is a solution suitable for intratumoral injection.
E73. The method of embodiment 72, wherein the solution comprises a buffer.
E74. The method of any one of the preceding embodiments, wherein the treatment results in an anti-tumor immune response in the patient comprising T cell activation, T cell proliferation, and/or T cell expansion.
E75. The method of embodiment 74, wherein the T cells are CD4+ T cells, CD8+ T cells, or both CD4+ T cells and CD8+ T cells.
E76. The method of any one of the preceding embodiments, wherein treatment results in a reduction in size or inhibition of growth of the injected tumor.
E77. The method of any one of the preceding embodiments, wherein treatment results in a reduction in size or inhibition of growth of an uninjected tumor.
E78. The method of any one of the preceding embodiments, wherein treatment results in an increase in expression of IL-23 and/or IL-36γ in the plasma and/or tumor of the patient.
E79. The method of any one of the preceding embodiments, wherein treatment results in an increase in expression of IL-22, IL-6, TNFα, IFNγ and any combination thereof in the plasma and/or tumor of the patient.
E80. The method of any one of the preceding embodiments, wherein treatment results in an increase in PD-L1 expression in tumor cells and/or immune cells within the tumor microenvironment.
E81. An LNP encapsulated mRNA therapeutic agent for use in the manufacture of a medicament for treating advanced or metastatic solid tumor malignancy or lymphoma in a human patient by inducing or enhancing an anti-tumor immune response, wherein the medicament comprises the LNP encapsulated mRNA therapeutic agent, and an optional pharmaceutically acceptable carrier, and wherein the treatment comprises administration of the medicament by intratumoral injection at a dose of 0.10-10.0 mg of the mRNA therapeutic agent in a dosing regimen from 7 to 28 days, and wherein the LNP encapsulated mRNA therapeutic agent comprises three mRNA drug substances:

(i) a first mRNA comprising an ORF encoding a human OX40L polypeptide;

(ii) a second mRNA comprising an ORF encoding a human IL-23 polypeptide; and

(iii) a third mRNA comprising an ORF encoding a human IL-36γ polypeptide.

E82. A kit comprising a container comprising a pharmaceutical composition comprising: an LNP encapsulated mRNA therapeutic agent; and a pharmaceutically acceptable carrier, and instructions for use in treating advanced or metastatic solid tumor malignancy or lymphoma in a human patient by inducing or enhancing an anti-tumor immune response, wherein the treatment comprises administration of the pharmaceutical composition by intratumoral injection at a dose of 0.10-10.0 mg of the mRNA therapeutic agent in a dosing regimen from 7 to 28 days, and wherein the LNP encapsulated mRNA therapeutic agent comprises three mRNA drug substances:

(i) a first mRNA comprising an ORF encoding a human OX40L polypeptide;

(ii) a second mRNA comprising an ORF encoding a human IL-23 polypeptide; and

(iii) a third mRNA comprising an ORF encoding a human IL-36γ polypeptide.

E83. The use of embodiment 81 or the kit of embodiment 82, wherein treatment comprises administration of the medicament at a dose of the mRNA therapeutic agent selected from: 0.25-8.0 mg; 0.25-4.0 mg; 0.25-2.0 mg; 0.25-1.0 mg; 0.25-5 mg; 0.5-8.0 mg; 0.5-4.0 mg; 0.5-2.0 mg; 0.5-1.0 mg; 1.0-8.0 mg; 1.0-4.0 mg; 1.0-2.0 mg; 2.0-8.0 mg; 2.0-4.0 mg; and 4.0-8.0 mg.
E84. The use of embodiment 81 or the kit of embodiment 82, wherein treatment comprises administration of the medicament at a dose of the mRNA therapeutic agent selected from 0.10 mg, 0.25 mg, 0.50 mg, 1.0 mg, 2.0 mg, 4.0 mg, 8.0 mg and 10.0 mg.
E85. The use or kit of any one of embodiments 81-84, wherein treatment comprises administration of the medicament to the patient in a dosing regimen selected from 7 to 21 days, 7 to 14 days, 28 days, 21 days, 14 days, and 7 days.
E86. The use or kit of any one of embodiments 81-85, wherein treatment comprises administration of the medicament every 2 weeks.
E87. The use or kit of any one of embodiments 81-85, wherein treatment comprises administration of the medicament every 3 weeks.
E88. The use or kit of any one of embodiments 81-85, wherein treatment comprises administration of the medicament every 4 weeks.
E89. The use or kit of any one of embodiments 81-85, wherein treatment comprises administration of the medicament in a first dosing cycle of every 2 weeks, and at least one subsequent dosing cycle of every 4 weeks.
E90. The use or kit of any one of embodiments 81-85, wherein treatment comprises administration of the medicament in a first dosing cycle of every 2 weeks, and at least three subsequent dosing cycles of every 4 weeks.
E91. The use or kit of any one of embodiments 81-90, wherein treatment comprises administration of the medicament or pharmaceutical composition in combination with a composition comprising a PD-1 antagonist, a PD-L1 antagonist, or a CTLA-4 antagonist, and an optional pharmaceutically acceptable carrier.
E92. The use or kit of embodiment 91, wherein the PD-1 antagonist is an antibody or antigen binding portion thereof that specifically binds to PD-1.
E93. The use or kit of embodiment 91, wherein the PD-L1 antagonist is an antibody or antigen binding portion thereof that specifically binds to PD-L1.
E94. The use or kit of embodiment 91, wherein the CTLA-4 antagonist is an antibody or antigen binding portion thereof that specifically binds to CTLA-4.
E95. The use or kit of embodiment 92, wherein the PD-1 antagonist is selected from the group consisting of nivolumab, pembrolizumab, and pidilizumab.
E96. The use or kit of embodiment 93, wherein the PD-L1 antagonist is selected from the group consisting of durvalumab, avelumab, and atezolizumab.
E97. The use or kit of embodiment 96, wherein the PD-L1 antagonist is durvalumab.
E98. The use or kit of embodiment 94, wherein the CTLA-4 antagonist is selected from the group consisting of ipilimumab and tremelimumab.
E99. The use or kit of embodiment 98, wherein the CTLA-4 antagonist is tremelimumab.
E100. The use or kit of any one of embodiments 91-99, wherein treatment comprises administration of a dose of the composition comprising the PD-1 antagonist, the PD-L1 antagonist or the CTLA-4 antagonist every 4 weeks.
E101. The use or kit of any one of embodiments 91-100, wherein treatment comprises administration of the medicament comprising an LNP encapsulated mRNA therapeutic agent prior to administration of the PD-1 antagonist, PD-L1 antagonist or CTLA-4 antagonist.
E102. The use or kit of any one of embodiments 81-101, wherein the advanced or metastatic solid tumor malignancy in the patient is selected from triple negative breast cancer, head and neck squamous cell carcinoma, and melanoma and the lymphoma is Non-Hodgkin lymphoma.
E103. The use or kit of any one of embodiments 81-102, wherein the human OX40L polypeptide comprises the amino acid sequence as set forth in SEQ ID NO: 1; the human IL-23 polypeptide comprises the amino acid sequence as set forth in SEQ ID NO: 24; and the human IL-36γ polypeptide comprises the amino acid sequence as set forth in SEQ ID NO: 27.
E104. The use or kit of any one of embodiments 81-102, wherein

(i) the first mRNA encoding a human OX40L polypeptide comprises an ORF comprising a nucleotide sequence at least 80% identical to the nucleotide sequence set forth in SEQ ID NO: 4 or comprises the nucleotide sequence set forth in SEQ ID NO: 4;

(ii) the second mRNA encoding a human IL-23 polypeptide comprises an ORF comprising a nucleotide sequence at least 80% identical to the nucleotide sequence set forth in SEQ ID NO: 25 or comprises the nucleotide sequence set forth in SEQ ID NO: 25; and

(iii) the third mRNA encoding a human IL-36γ polypeptide comprises an ORF comprising a nucleotide sequence at least 80% identical to the nucleotide sequence set forth in SEQ ID NO: 28 or comprises the nucleotide sequence set forth in SEQ ID NO: 28.

E105. The use or kit of any one of embodiments 81-104, wherein each of the first mRNA, second mRNA, and third mRNA comprise a 3′ untranslated region (UTR) comprising at least one microRNA-122 (miR-122) binding site.
E106. The use or kit of embodiment 105, wherein the miR-122 binding site is a miR-122-3p binding site or a miR-122-5p binding site.
E107. The use or kit of embodiment 106, wherein the miR-122-5p binding site comprises the nucleotide sequence set forth in SEQ ID NO: 20.
E108. The use or kit of embodiment 105, wherein the 3′UTR comprises a nucleotide sequence at least 90% identical to the sequence set forth in SEQ ID NO: 17 or comprises the nucleotide sequence as set forth in SEQ ID NO: 17.
E109. The use or kit of any one of embodiments 81-108, wherein each of the first, second, and third mRNAs comprise a 5′cap, a 5′ untranslated region (UTR), and a poly-A tail of about 100 nucleotides in length.
E110. The use or kit of embodiment 109, wherein the 5′UTR comprises a nucleotide sequence at least 90% identical to the sequence set forth in SEQ ID NO: 16 or comprises the nucleotide sequence as set forth in SEQ ID NO: 16.
E111. The use or kit of any one of embodiments 81-102, wherein

(i) the first mRNA encoding the human OX40L polypeptide comprises a nucleotide sequence at least 90% identical to the nucleotide sequence set forth in SEQ ID NO: 5 or comprises the nucleotide sequence set forth in SEQ ID NO: 5;

(ii) the second mRNA encoding a human IL-23 polypeptide comprises a nucleotide sequence at least 90% identical to the nucleotide sequence set forth in SEQ ID NO: 26 or comprises the nucleotide sequence set forth in SEQ ID NO: 26; and

(iii) the third mRNA encoding a human IL-36γ polypeptide comprises a nucleotide sequence at least 90% identical to the nucleotide sequence set forth in SEQ ID NO: 29 or comprises the nucleotide sequence set forth in SEQ ID NO: 29.

E112. The use or kit of any one of embodiments 81-111, wherein the first, second, and third mRNAs are formulated in the lipid nanoparticle at a mass ratio of OX40L:IL-23:IL-36γ of 1:1:2.
E113. The use or kit of any one of embodiments 81-112, wherein each of the first, second and third mRNAs is chemically modified.
E114. The use or kit of embodiment 113, wherein each of the first, second, and third mRNAs is fully modified with chemically-modified uridines.
E115. The use or kit of embodiment 114, wherein the chemically-modified uridines are N1-methylpseudouridines (m1ψ).
E116. The use or kit of embodiment 113, wherein each of the first, second and third mRNAs is fully modified with 5-methylcytosine or is fully modified with N1-methylpseudouridines (m1ψ) and 5-methylcytosine.
E117. The use or kit of any one of embodiments 81-116, wherein the LNP comprises a compound having the formula:

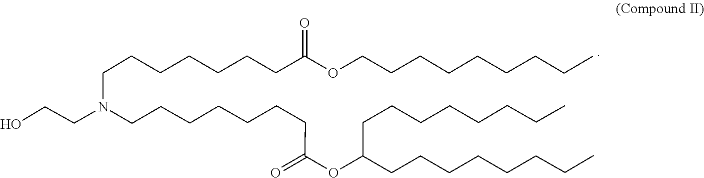 [SMILES: CCCCCCCCCOC(=O)CCCCCCCN(CCO)CCCCCCCC(=O)OC(CCCCCCCC)CCCCCCCC]

E118. The use or kit of embodiment 117, wherein the LNP further comprising a phospholipid, a structural lipid, and a PEG lipid.
E119. The use or kit of embodiment 118, wherein the LNP comprises a molar ratio of about 20-60% ionizable amino lipid, about 5-25% phospholipid, about 25-55% structural lipid, and about 0.5-1.5% PEG lipid.
E120. The use or kit of embodiment 118, wherein the LNP comprises a molar ratio of about 50% ionizable amino lipid, about 10% phospholipid, about 38.5% structural lipid, and about 1.5% PEG lipid.
E121. The use or kit of embodiment 118, wherein the LNP comprises a molar ratio of about 50% ionizable amino lipid, about 10% phospholipid, about 38.5% cholesterol, and about 1.5% PEG-DMG.

### Definitions

Abscopal effect: As used herein, “abscopal effect” refers to a phenomenon in the treatment of cancer, including metastatic cancer, where localized administration of a treatment (e.g., mRNAs encoding OX40L, IL-23 and IL-36γ) to a tumor causes not only a reduction in size of the treated tumor but also a reduction in size of tumors outside the treated area. In some embodiments, the abscopal effect is a local, regional abscopal effect, wherein a proximal or nearby tumor relative to the treated tumor is affected. In some embodiments, the abscopal effect occurs in a distal tumor relative to the treated tumor. In some embodiments, treatment (e.g., mRNAs encoding OX40L, IL-23 and IL-36γ) is administered via intratumoral injection, resulting in a reduction in tumor size of the injected tumor and a proximal or distal uninjected tumor.

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation, as an oral spray and/or powder, nasal spray, and/or aerosol, and/or through a portal vein catheter.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

CPI-refractory: As used herein, the term “CPI-refractory” refers to a cancer in a human patient that fails to respond to immune checkpoint inhibitor (CPI) therapy. In some embodiments, CPI-refractory is primary, such that a cancer progresses in the patient after exposure to CPI therapy. In some embodiments, CPI-refractory is secondary acquired, such that a cancer in the patient initially responds to CPI therapy or stable disease is observed, followed by cancer progression in the patient in a setting of ongoing CPI therapy.

Conjugated: As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., an isolated mRNA, nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle or an isolated mRNA) and a cell may be contacted, for example, by adding the composition to the culture medium of the cell and may involve or result in transfection. Moreover, more than one cell may be contacted by a nanoparticle composition.

Dosing cycle: As used herein, the term “dosing cycle” refers to a discrete amount of time, expressed in units of time, during which at least one dose is administered. In some embodiments, a dosing interval occurs during a dosing cycle. For example, in some embodiments, a dosing interval of every two weeks occurs during a dosing cycle of 4 weeks.

Dosing interval: As used herein, the term “dosing interval”, “dosage interval” or “dosing regimen” refers to a discrete amount of time, expressed in units of time, (e.g., 14 days) that transpires between individual administrations (plural) of a dose of a therapeutic composition (e.g., a composition comprising an mRNA). For example, in some embodiments, a dosing interval starts on the day a first dose is administered (e.g., initial dose), and ends on the day a second dose (e.g., a subsequent dose) is administered. In some embodiments, there are multiple dosing intervals during treatment.

Encapsulate: As used herein, the term “encapsulate” means to enclose, surround, or encase. In some embodiments, a compound, an mRNA, or other composition may be fully encapsulated, partially encapsulated, or substantially encapsulated. For example, in some embodiments, an mRNA of the disclosure may be encapsulated in a lipid nanoparticle, e.g., a liposome.

Effective amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent. In some embodiments, a therapeutically effective amount is an amount of an agent to be delivered (e.g., nucleic acid, drug, therapeutic agent, diagnostic agent or prophylactic agent) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Fragment: A “fragment,” as used herein, refers to a portion. For example, fragments of proteins may include polypeptides obtained by digesting full-length protein isolated from cultured cells or obtained through recombinant DNA techniques.

Identity: As used herein, the term “identity” refers to the overall relatedness between polymeric molecules, e.g., between nucleic acid molecules (e.g., DNA molecules and/or RNA molecules) and/or between polypeptide molecules. Calculation of the percent identity of two mRNA sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux et al., Nucleic Acids Research, 12(1): 387, 1984, BLASTP, BLASTN, and FASTA, Altschul, S. F. et al., J. Molec. Biol., 215, 403, 1990.

Immune checkpoint inhibitor: An “immune checkpoint inhibitor” or simply “checkpoint inhibitor” refers to a molecule that prevents immune cells from being turned off by cancer cells. As used herein, the term checkpoint inhibitor refers to polypeptides (e.g., antibodies) or mRNAs encoding such polypeptides that neutralize or inhibit inhibitory checkpoint molecules such as cytotoxic T-lymphocyte-associated protein 4 (CTLA-4), programmed death 1 receptor (PD-1), or PD-1 ligand 1 (PD-L1).

Immune response: The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues. In some cases, the administration of a nanoparticle comprising a lipid component and an encapsulated therapeutic agent can trigger an immune response, which can be caused by (i) the encapsulated therapeutic agent (e.g., an mRNA), (ii) the expression product of such encapsulated therapeutic agent (e.g., a polypeptide encoded by the mRNA), (iii) the lipid component of the nanoparticle, or (iv) a combination thereof.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Linker: As used herein, a “linker” (including a subunit linker, and a heterologous polypeptide linker as referred to herein) refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker can be of sufficient length as to not interfere with incorporation into a nucleic acid sequence. The linker can be used for any useful purpose, such as to form polynucleotide multimers (e.g., through linkage of two or more chimeric polynucleotides molecules or IVT polynucleotides) or polynucleotides conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof. Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

Metastasis: As used herein, the term “metastasis” means the process by which cancer spreads from the place at which it first arose as a primary tumor to distant locations in the body. A secondary tumor that arose as a result of this process may be referred to as “a metastasis.”

mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5 cap and a polyA sequence.

microRNA (miRNA): As used herein, a “microRNA (miRNA)” is a small non-coding RNA molecule which may function in post-transcriptional regulation of gene expression (e.g., by RNA silencing, such as by cleavage of the mRNA, destabilization of the mRNA by shortening its polyA tail, and/or by interfering with the efficiency of translation of the mRNA into a polypeptide by a ribosome). A mature miRNA is typically about 22 nucleotides long.

microRNA-122 (miR-122): As used herein, “microRNA-122 (miR-122)” refers to any native miR-122 from any vertebrate source, including, for example, humans, unless otherwise indicated. miR-122 is typically highly expressed in the liver, where it may regulate fatty-acid metabolism. miR-122 levels are reduced in liver cancer, for example, hepatocellular carcinoma. miR-122 is one of the most highly-expressed miRNAs in the liver, where it regulates targets including but not limited to CAT-1, CD320, AldoA, Hjv, Hfe, ADAM10, IGFR1, CCNG1, and ADAM17. Mature human miR-122 may have a sequence of AACGCCAUUAUCACACUAAAUA (SEQ ID NO: 73, corresponding to hsa-miR-122-3p) or UGGAGUGUGACAAUGGUGUUUG (SEQ ID NO: 82, corresponding to hsa-miR-122-5p).

microRNA (miRNA) binding site: As used herein, a “microRNA (miRNA) binding site” refers to a miRNA target site or a miRNA recognition site, or any nucleotide sequence to which a miRNA binds or associates. In some embodiments, a miRNA binding site represents a nucleotide location or region of an mRNA to which at least the “seed” region of a miRNA binds. It should be understood that “binding” may follow traditional Watson-Crick hybridization rules or may reflect any stable association of the miRNA with the target sequence at or adjacent to the microRNA site.

miRNA seed: As used herein, a “seed” region of a miRNA refers to a sequence in the region of positions 2-8 of a mature miRNA, which typically has perfect Watson-Crick complementarity to the miRNA binding site. A miRNA seed may include positions 2-8 or 2-7 of a mature miRNA. In some embodiments, a miRNA seed may comprise 7 nucleotides (e.g., nucleotides 2-8 of a mature miRNA), wherein the seed-complementary site in the corresponding miRNA binding site is flanked by an adenine (A) opposed to miRNA position 1. In some embodiments, a miRNA seed may comprise 6 nucleotides (e.g., nucleotides 2-7 of a mature miRNA), wherein the seed-complementary site in the corresponding miRNA binding site is flanked by an adenine (A) opposed to miRNA position 1. When referring to a miRNA binding site, an miRNA seed sequence is to be understood as having complementarity (e.g., partial, substantial, or complete complementarity) with the seed sequence of the miRNA that binds to the miRNA binding site.

Modified: As used herein “modified” refers to a changed state or structure of a molecule of the disclosure. Molecules may be modified in many ways including chemically, structurally, and functionally. In one embodiment, the mRNA molecules of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

Nanoparticle: As used herein, “nanoparticle” refers to a particle having any one structural feature on a scale of less than about 1000 nm that exhibits novel properties as compared to a bulk sample of the same material. Routinely, nanoparticles have any one structural feature on a scale of less than about 500 nm, less than about 200 nm, or about 100 nm. Also routinely, nanoparticles have any one structural feature on a scale of from about 50 nm to about 500 nm, from about 50 nm to about 200 nm or from about 70 to about 120 nm. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Neoadjuvant: As used herein, the term “neoadjuvant” refers to an initial treatment of a disease in a human patient, and thus a “neoadjuvant” cancer refers to a cancer in a patient that has not received prior treatment to a therapeutic of interest (e.g., the mRNA therapeutic agent described herein). The term “neoadjuvant melanoma” refers to a melanoma in a patient that has not received anti-cancer treatment prior to administration of the mRNA therapeutic agent described herein.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Operably linked: As used herein, the phrase “operably linked” refers to a functional connection between two or more molecules, constructs, transcripts, entities, moieties or the like.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from cancer (e.g., liver cancer or colorectal cancer).

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Polypeptide: As used herein, the term “polypeptide” or “polypeptide of interest” refers to a polymer of amino acid residues typically joined by peptide bonds that can be produced naturally (e.g., isolated or purified) or synthetically.

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a human patient having a solid tumor malignancy or lymphoma.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeting moiety: As used herein, a “targeting moiety” is a compound or agent that may target a nanoparticle to a particular cell, tissue, and/or organ type.

Therapeutic Agent: The term “therapeutic agent” refers to any agent that, when administered to a subject, has a therapeutic, diagnostic, and/or prophylactic effect and/or elicits a desired biological and/or pharmacological effect.

Treating: As used herein, the term “treating” refers to partially or completely alleviating, ameliorating, improving, relieving, delaying onset of, inhibiting progression of, reducing severity of, and/or reducing incidence of one or more symptoms or features of a solid tumor malignancy or lymphoma. For example, “treating” cancer may refer to inhibiting survival, growth, and/or spread of a tumor. Treatment may be measured by reduction in numbers of tumors or reduction in size of a particular tumor and/or reduction in metastasis. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Tumor: As used herein, a “tumor” is an abnormal growth of tissue, whether benign or malignant.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

### Equivalents and Scope

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

### Example 1: Synergistic Efficacy of Triple Combination of mRNAs Encoding OX40L, IL-23 and IL-36γ in the Immunosuppressive MC38-M Colon Cancer Model

The efficacy of triple combination therapy comprising mRNA encoding OX40L, mRNA encoding IL-23, and mRNA encoding IL-36γ was assessed in the MC38 colon cancer model.

mRNA encoding a mouse OX40L polypeptide (amino acid sequence SEQ ID NO: 3), mRNA encoding a human IL-23 polypeptide comprising IL-12p40 subunit and IL-23p19 subunit linked by GS Linker (amino acid sequence SEQ ID NO: 24), and mRNA encoding a human IL-36γ polypeptide (amino acid sequence SEQ ID NO: 27) were prepared. Each mRNA comprised a miR-122 binding site in the 3′UTR.

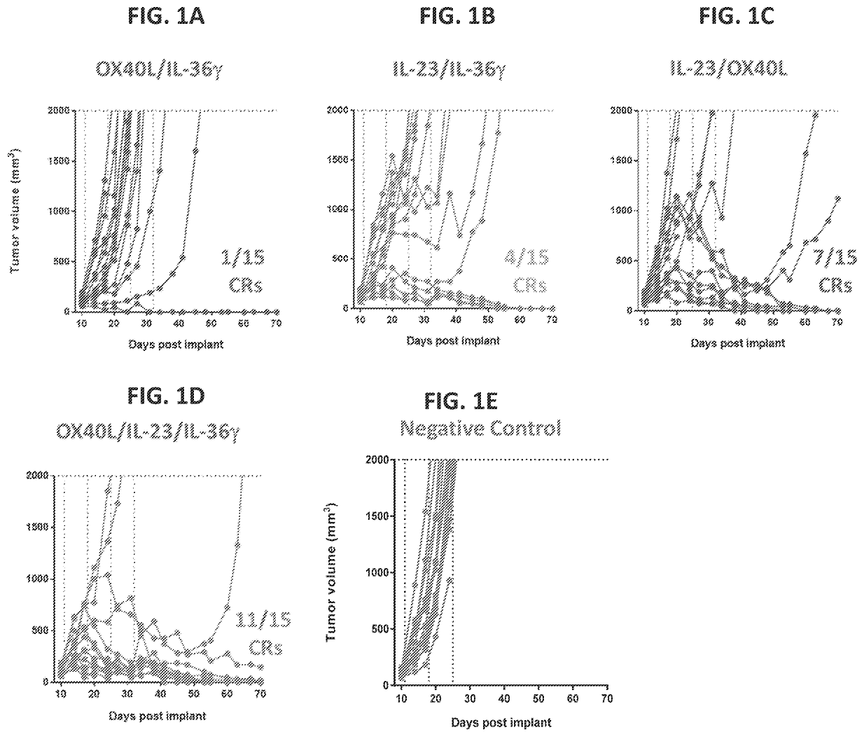

MC-38 colon adenocarcinoma tumors were established subcutaneously in C57BL/6 mice. See Rosenberg et al., Science 233(4770):1318-21 (1986). Tumors were monitored for size and palpability. See Kim et al., Journal of Immunology 122(2):549-554 (1979); Donnou et al., Advances in Hematology 2012:701704 (2012). Once the MC-38 tumors reached a mean size of approximately 100 mm³, animals were treated with single intratumoral doses of mRNAs formulated in Compound II-based lipid particles at 5 μg total mRNA/dose, every 7 days for 4 cycles (Q7Dx4). FIGS. 1A-1E, dashed vertical lines. The number of complete responders (CRs) are indicated, n/group=15.

Control animals were treated with an equivalent dose of negative control mRNA formulated in the same LNP. Negative controls were non-translatable versions of an mRNA encoding OX40L, wherein the mRNA comprises multiple stop codons.

The results demonstrate that intratumoral (ITU) administration of LNP comprising triplet combination of mRNAs encoding OX40L/IL-23/IL-36γ polypeptides achieved 11/15 CRs (FIG. 1D). This response rate is superior to combinations with doublet mRNAs. There was 1/15 CR in the group administered LNP comprising doublet mRNAs encoding OX40L/IL-36γ polypeptides (FIG. 1A). The groups administered LNP comprising doublet mRNAs encoding IL-23/IL-36γ and IL-23/OX-40L polypeptides have responder rates of 4/15 and 7/15, respectively. FIGS. 1B, 1C.

### Example 2: Marked Efficacy in Both Primary Treated and Untreated Distal Tumors with Triplet mRNA Therapy

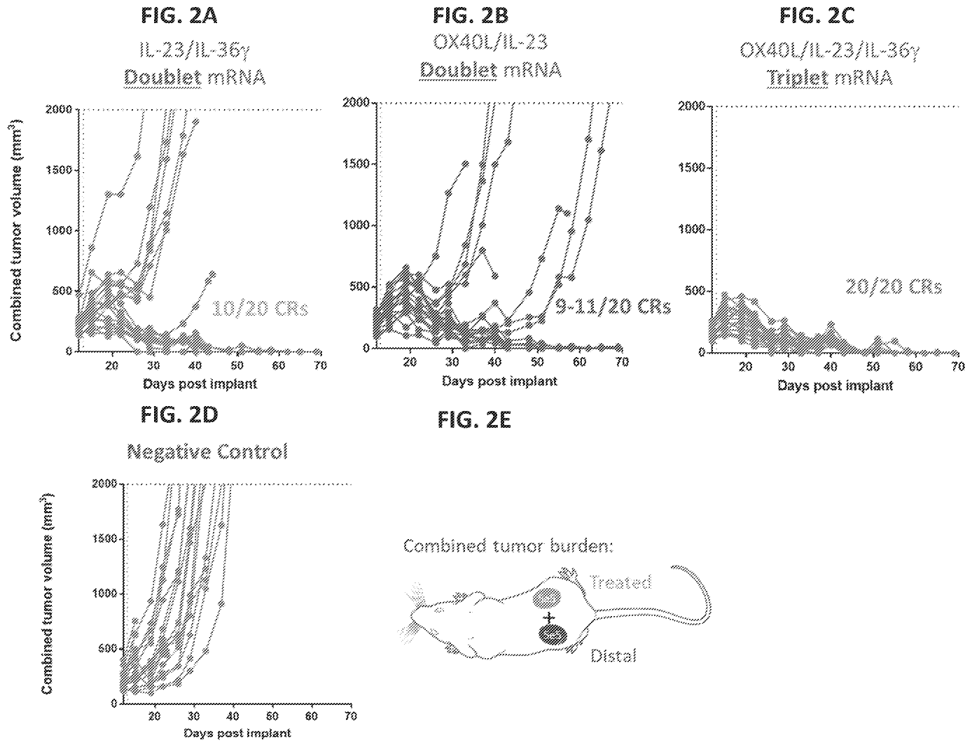

Experiments were conducted using the MC38-S dual flank mice tumor model and mRNA as described in Example 1. The MC38-S variant is less aggressive and allows multiple tumor modeling in single mice. MC38-S cells were implanted in each flank of each animal. See FIG. 2E. Only one tumor in each mice were treated, whereas a distal tumor was left untreated. A primary tumor on one flank was administered doublet combinations of mRNAs encoding IL-23/IL-36γ or OX40L/IL-23; a triplet combination of mRNAs encoding OX40L, IL-23, IL-36γ; or control mRNA (non-translating mRNA encoding for OX40L). FIGS. 2A-2D. The total dose of mRNA per treatment was 5 μg of mRNA. LNP encapsulated mRNAs were administered as single intratumoral doses. N/group=20.

The combined effect of ITu treatment on the first and second (untreated) tumor was measured as combined tumor volume. FIGS. 2A-2D.

FIG. 2D shows combined tumor volumes in mice treated with control mRNA. When doublet mRNA IL-23/IL-36γ or OX40L/IL-23 therapy were administered to the primary tumors, approximately 50% CRs were observed. (FIGS. 2A, 2B). When the triplet mRNA therapy was administered to the primary tumors (FIG. 2C), 20 complete responses (100%) were observed.

This data indicates that treatment of a tumor with an mRNA therapeutic composition can effectively treat tumors at other locations. As shown in FIG. 2C, a single, relatively low ITu dose of triplet mRNA was able to induce complete disease control in mice bearing two tumors at both treated and uninjected distal sites. The results demonstrate that local therapy is efficacious for treatment of multi-lesional and metastatic cancers. FIG. 2F shows the effect of doublet mRNA combinations and a triplet mRNA combination therapies on survival rate. Animals that were not treated or administered control mRNA did not survive past day 45 of the study. After day 70, 70% of the mice administered doublet mRNAs encoding OX40L/IL-23 or IL-23/IL-36γ survived implantation with MC38 tumors. All mice treated with triplet mRNA encoding OX40L/IL-23/IL-36γ achieved complete response and survived implantation with MC8 tumors. Thus local therapy with triplet mRNA was able to achieve complete disease control, and is superior to doublet mRNA combinations.

### Example 3: Efficacy of a Combination Treatment Comprising Triplet mRNA Therapy and Anti-PD-L1 Antibodies in MC38 Model

The administration of doublet and triplet therapy increased levels of PD-L1. Slight increases in PD-L1 levels were observed in cancer cells, e.g., CD45−, FSC-hi and MHCII−, after the administration of triplet therapy. The administration of the doublet IL-23/IL-36γ also resulted in an increased percentage of CD11b+ cells positive for PD-L1. This observation correlated with an increase in PD-L1 expression in CD11b+ cells. Administration of the triplet combination also resulted in an increased percentage of CD11b+ cells positive for PD-L1 and an increase in PD-L1 expression in CD11b+ cells. Data not shown.

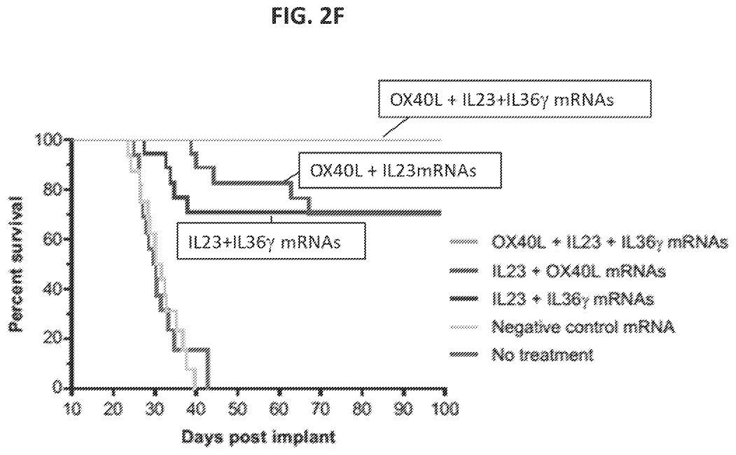

The increase in expression of PD-L1 in an immunosuppressive MC38 model in response to treatment with triplet mRNA therapy provided a rationale to combine the triplet therapy with anti-PD-L1 antibodies. Total mRNA dosing was 5 μg of total mRNA, administered intratumorally as a single dose of LNP encapsulated mRNAs as described in Example 1. FIGS. 3B, 3C, 3D. The antibody (anti-PD-L1 antibody 10F.9G2 or control) was dosed intraperitoneally twice per week at 10 mg/kg. FIGS. 3A, 3B, 3C. N/group=15.

No responses were observed when the negative control mRNA (FIG. 3D) or the anti-PD-L1 antibody (FIG. 3A) were administered alone. When the triplet mRNA therapy (mRNAs encoding OX40L/IL-23/IL-36γ) was administered, 3-4 out of 15 mice showed CRs. FIG. 3B. On the other hand, when the triplet mRNA therapy was administered in combination with the anti-PD-L1 antibody, 9-10 out of 15 mice experienced complete responses. FIG. 3C. In a follow-up study with anti-PD-L1 antibody in combination with triplet mRNA, 12/15 CRs were observed. Data not shown. Synergy (12/15 CRs) was also observed with anti-PD-1 antibody combination with triplet mRNA. Data not shown. These data indicate that tumors refractory to treatment with a systemic conventional therapy, e.g., an anti-PD-L1 antibody, can be effectively treated by combining such therapy with a triple therapy comprising mRNAs encoding OX40L/IL-23/IL-36γ.

### Example 4: Efficacy of a Combination Treatment Comprising Triplet mRNA Therapy and Anti-CTLA4 Antibodies in MC38 Model

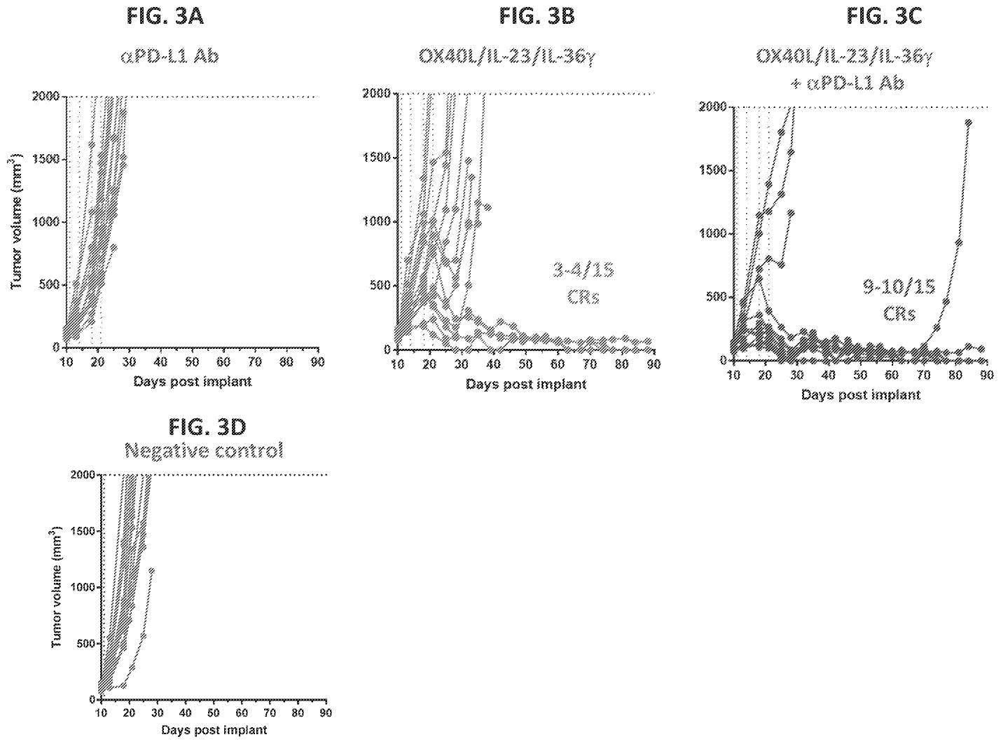

The efficacy of a combination of the triplet therapy with anti-CTLA4 antibodies was assessed in the immunosuppressive MC38 model. Total mRNA dosing was 5 μg of total mRNA, administered intratumorally as a single dose of LNP encapsulated mRNAs as described in Example 1. FIGS. 4B, 4C,4D. The antibody (anti-CTLA4 antibody 9D9 or control) was dosed intraperitoneally twice per week at 10 mg/kg. FIGS. 4A, 4B, 4C. N/group=15.

No responses were observed when the negative control mRNA (FIG. 4D) or the anti-CTLA-4 antibody (FIG. 4A) were administered alone. When the triplet mRNA therapy (mRNAs encoding OX40L/IL-23/IL-36γ) was administered, 6 out of 15 mice showed CRs. FIG. 4B. On the other hand, when the triplet mRNA therapy was administered in combination with the anti-CTLA-4 antibody, 12 out of 15 mice experienced complete responses. FIG. 4C. These data indicate that tumors refractory to treatment with a systemic conventional therapy, e.g., an anti-CTLA-4 antibody, can be effectively treated by combining such therapy with a triple therapy comprising mRNAs encoding OX40L/IL-23/IL-36γ. Further, the synergistic efficacy of triplet mRNA therapy is shown with checkpoint inhibitors that target biologically distinct (anti-PD-L1 and anti-CTLA4) pathways. FIGS. 3C, 4C.

### Example 5: Clinical Study Design to Evaluate Anti-Tumor Efficacy of mRNAs Encoding OX40L, IL-23 and IL-36γ Alone or in Combination with Immune Checkpoint Blockade

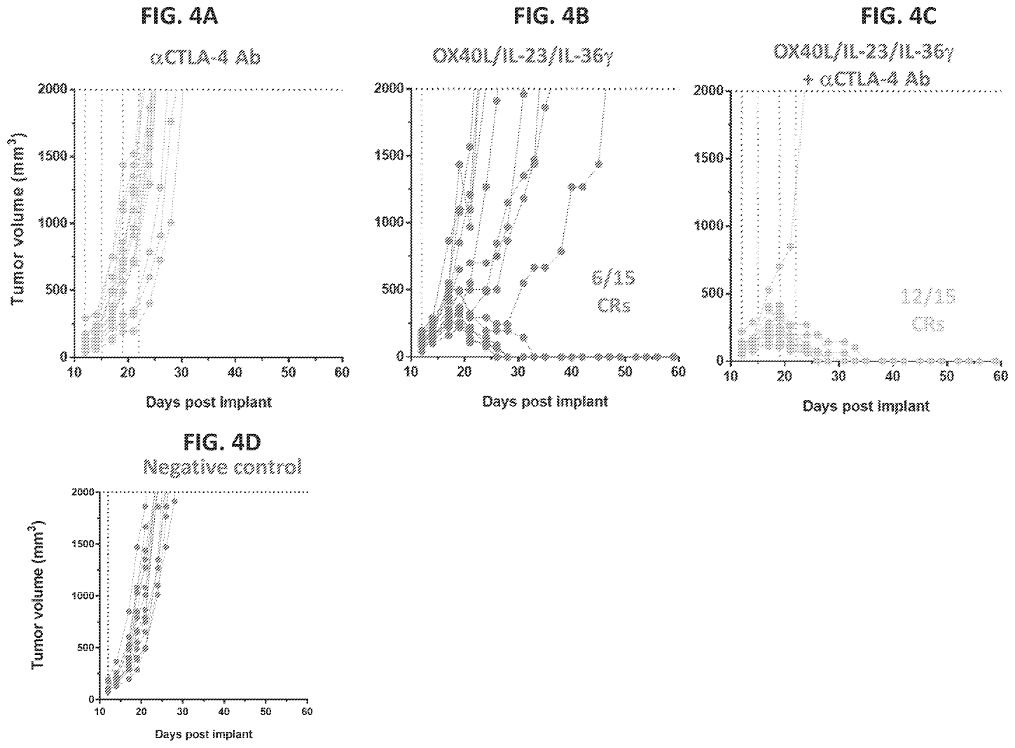

FIG. 5 is the study design of a Phase 1, dose escalation study of ITu injections of mRNA-TRIPLET comprising mRNAs encoding polypeptides of OX40L, IL-23 and IL-36γ alone and in combination with intravenously administered immune checkpoint blockade therapy in patients with histologically confirmed advanced or metastatic solid tumor malignancies or lymphomas. The study includes the following three treatment arms:

Arm A: mRNA-TRIPLET alone, administered every two weeks (Q2W) for 3 doses.

Arm B: mRNA-TRIPLET in combination with durvalumab (PD-L1 inhibitor) every two weeks (Q2W) for the first cycle, and every four weeks (Q4W) for cycles 2-6. Following completion of 3 cycles of mRNA-TRIPLET, patients may continue with durvalumab as a single-agent until disease progression, unacceptable toxicity, or total of twenty-four months of treatment, whichever is sooner.

Arm C: mRNA-TRIPLET in combination with tremelimumab (CTLA-4 inhibitor) Q4W for three cycles. Following completion of 3 cycles of mRNA-TRIPLET, patients may continue with tremelimumab as a single-agent for one additional cycle (4 cycles total) until disease progression or unacceptable toxicity, whichever is sooner.

The study comprises 3 dose escalation and dose confirmation parts (Arms A, B, and C) followed by a dose expansion part in select indications for combination Arms B and C. The dose expansion will comprise four treatment groups across the treatment arms summarized as follows:

Arm B: mRNA-TRIPLET in combination with durvalumab
Group 1: Triple-negative breast cancer (TNBC)
Group 2: Head and neck squamous cell carcinoma (HNSCC)
Group 3: Non-Hodgkin lymphoma (NHL)
Group 4: Urothelial cancer in patients that are ineligible for platinum-containing chemotherapy and are PD-L1 negative (1 L)
Group 5: Urothelial cancer in patients having objective evidence of disease progression during or following platinum-containing chemotherapy (2 L+)

Arm C: mRNA-TRIPLET in combination with tremelimumab
Group 6: Melanoma

Dose escalation will be conducted in patients with solid tumors or lymphoma with cutaneous or subcutaneous accessible lesions, and will start with mRNA-TRIPLET alone (Arm A). Once the first 2 dose levels of mRNA-TRIPLET alone (Arm A) are cleared for safety, dose escalation for mRNA-TRIPLET in combination with fixed dose of durvalumab (Arm B) will start. Once the first dose level of Arm B is cleared for safety, dose escalation for mRNA-TRIPLET in combination with a fixed dose of tremelimumab (Arm C) will start.

The dose levels for mRNA-TRIPLET for all treatment arms is as in the following table:

| Dose Level | mRNA-TRIPLET Dose | |
|---|---|---|
| −1* | 0.10 | mg |
| 1 | 0.25 | mg |
| (starting dose) | | |
| 2 | 0.50 | mg |
| 3 | 1 | mg |
| 4 | 2 | mg |
| 5 | 4 | mg |
| 6 | 8 | mg |

*Represents a treatment dose if de-escalation from the starting dose is required.

The dose and treatment schedule for the different study treatments are summarized in the following table:

| | | | |
|---|---|---|---|
| | Pharmaceutical Form and | | |
| Study Drugs | Route of Administration | Dose | Frequency |

| | | | | |
|---|---|---|---|---|
| mRNA-TRIPLET | Solution for ITu injection | 0.25 | mg | Q2W (Arm A and |

| | |
|---|---|
| (starting dose) | first cycle of Arm B) |
| | Q4W (cycles 2-6 of |
| | Arm B, and Arm C) |

| | | | | |
|---|---|---|---|---|
| Durvalumab | 500 mg vial solution for | 1500 | mg | Q4W |
| | infusion after dilution, 50 | | | |
| | mg/mL | | | |
| Tremelimumab | 400 mg vial solution for | 225 | mg | Q4W for 4 cycles |
| | infusion after dilution, 20 | | | |
| | mg/mL | | | |

Once the maximum tolerated dose (MTD) and/or recommended dose for expansion (RDE) have been determined in the dose escalation parts, dose confirmation of MTD and/or RDE for each treatment arm will be conducted with solid tumors or lymphoma with visceral lesions injectable with ultrasound or CT guidance.

Once the MTD and/or RDE have been determined in the dose escalation/dose confirmation parts, patients will be enrolled in the respective dose-expansion parts in order to assess the preliminary anti-tumor activity of mRNA-TRIPLET in combination with either durvalumab or tremelimumab in select indications. FIG. 5.

### Example 6: Initial Results of Clinical Study for mRNA-TRIPLET Alone or in Combination with Durvalumab

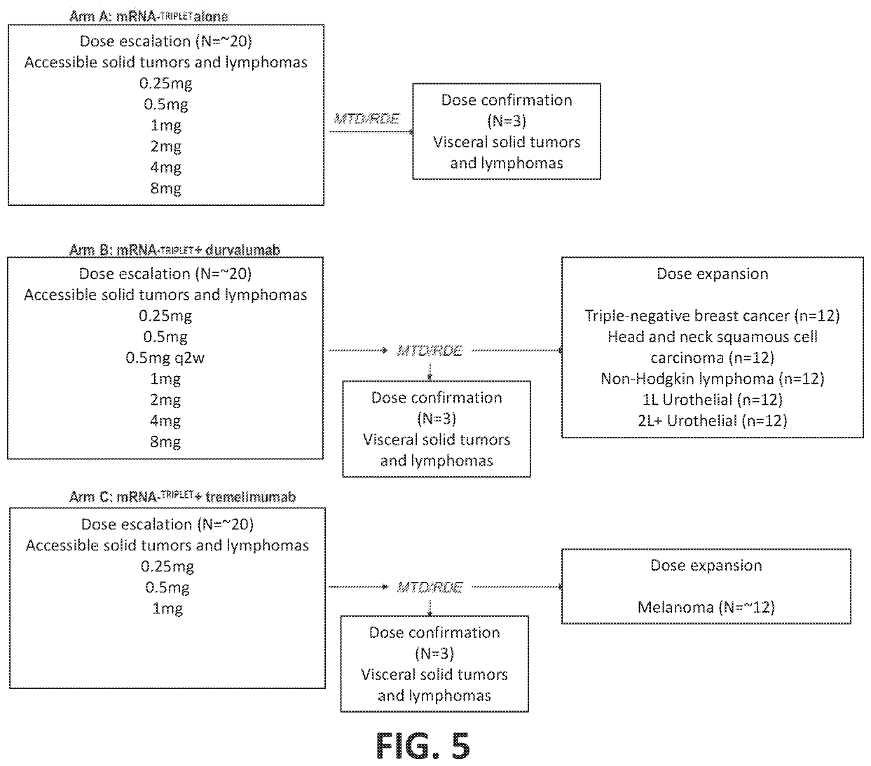

This example provides initial results from the Phase I, open-label, multicenter, dose escalation study described in Example 5. The dosing schedule for Arms A and B are shown in FIG. 6. Data for 21 patients (17 treated with monotherapy and 12 treated with combination therapy) was collected. Patient characteristics are shown in the below table:

| Arm A Monotherapy | | Arm B Combination | |
|---|---|---|---|
| No. of patients | 17 | No. of patients | 12 |
| Age (y) | | Age(y) | |
| Range: | 43-79 | Range: | 37-79 |
| Median: | 63 | Median: | 61.5 |
| Sex | | Sex | |
| Male: | 6 | Male: | 1 |
| Female: | 11 | Female: | 11 |
| Race | | Race | |
| Caucasian: | 14 | Caucasian: | 10 |
| Black: | 2 | Black: | 1 |
| Other: | 1 | Other: | 1 |
| Tumor Types | | Tumor Types | |
| Breast: | 5 | Breast: | 4 |
| Melanoma: | 5 | Bladder | 1 |
| | | (squamous): | |
| Colon | 2 | Endometrial: | 1 |
| Head and Neck: | 1 | Esophageal: | 1 |
| Ovarian: | 1 | Head and Neck: | 1 |
| Pancreatic: | 1 | Ovarian: | 1 |
| Other: | 2 | Sarcoma: | 2 |
| | | Other: | 1 |

### Disease Progression and Tumor Size

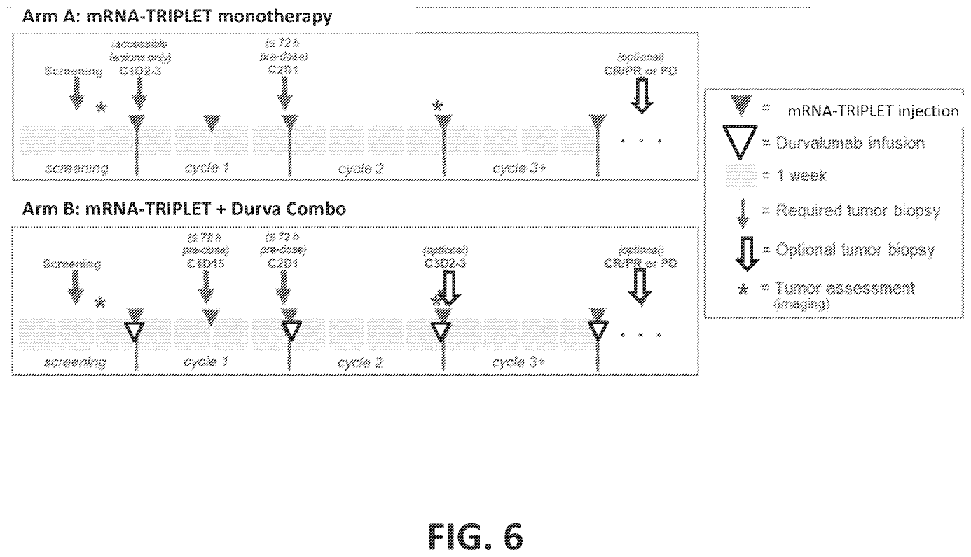

Study treatment has been well tolerated through all tested dose levels, with no Dose Limiting Toxicities or G3 toxicities related to treatment observed. Of the 23 patients evaluated per RECIST 1.1 and iRECIST, there was one partial response in Arm B; stable disease in 4 patients in Arm A and 5 patients in Arm B; and progressive disease in 10 patients in Arm A and 3 patients in Arm B (FIG. 7). The 13 patients with progressive disease came off the study.

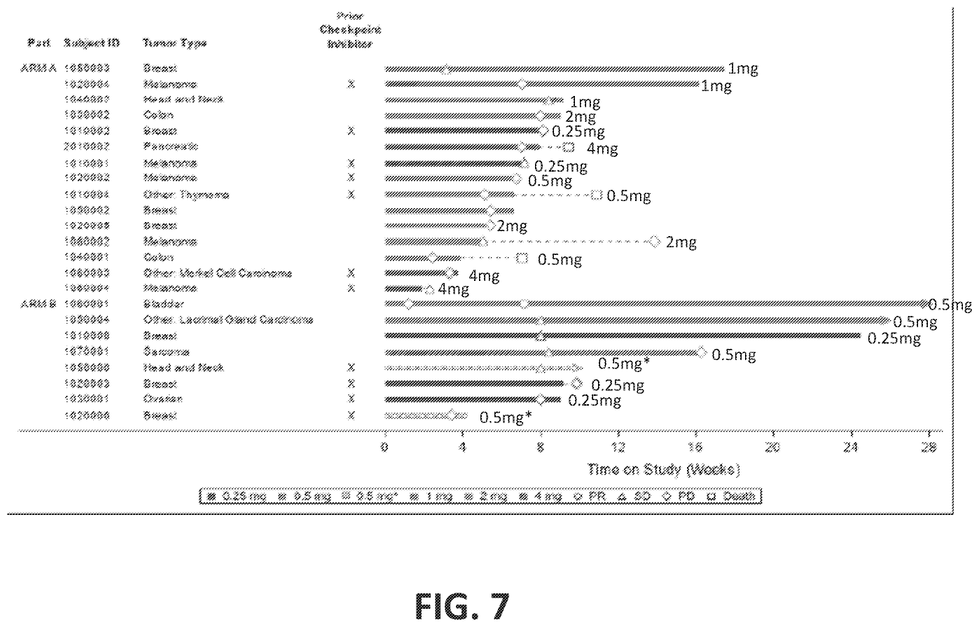

The partial response was an 81% shrinkage in target lesions observed in a PD-1/L1 naïve squamous-cell bladder patient receiving 0.5 mg mRNA-TRIPLET Q4W in combination with durvalumab (FIG. 8). Squamous-cell bladder cancer is a distinct subtype with unknown checkpoint inhibitor response rate, as it does not fall under the classification of urothelial bladder cancer, which is an approved indication for durvalumab. The patient has remained on the study.

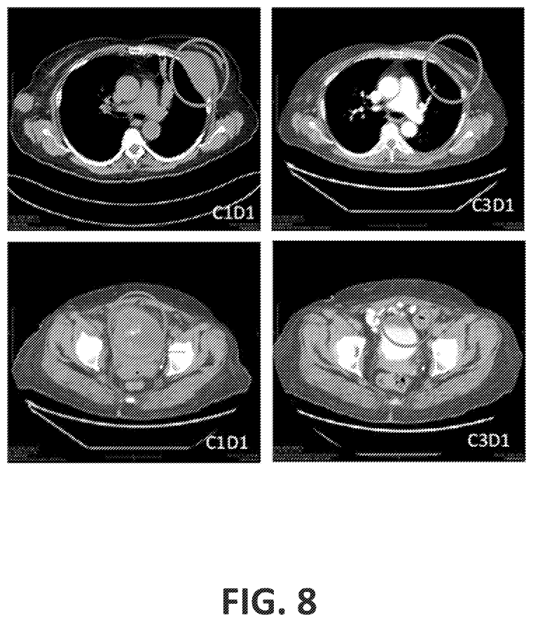

Tumor shrinkage was also observed in 7 patients in injected and/or uninjected lesions, as shown in FIG. 9. This was determined based on investigator assessment per RECIST 1.1.

### Cytokine Analysis

In addition to assessing the effect of treatment on disease progression and tumor size, biopsy samples were collected for biomarker analysis. Specifically, biopsy samples were collected during screening as set forth in FIG. 6. Each biopsy collection was split to be processed by (1) flash freezing for cytokine testing and (2) formalin-fixation and paraffin embedding (FFPE) for tumor microenvironment (TME) evaluations by PD-L1 SP263 immunohistochemistry (durvalumab complementary diagnostic; the SP263 antibody clone recognizes an intracellular PD-L1 epitope distinct from the extracellular epitope recognized by durvalumab) and H+E (Hematoxylin and Eosin) staining in the current dataset.

Initial plasma samples evaluated included those collected at baseline versus post-dose at 3 hours, 6 hours, 24 hours and 1 week. Plasma and frozen tumor lysates were evaluated by IL-36γ ELISA and multiplexed electrochemiluminescence assays (Th17 panel: IL-23, IL-22, IL-21, IL-17A, IL-27, IL-31, MIP3α (CCL20), and pro-inflammatory panel: IFN-γ, IL-2, IL-4, IL-6, IL-8, IL-10, IL-12p70, IL-13, IL-1β, TNF-α). Tumor cytokine levels were normalized to total protein concentration of each tumor lysate. In tissue samples, upper limit of quantification (ULOQ) values per assay were converted from pg/mL to pg/mg and plotted for results falling above the limit of quantification (ALOQ). Below limit of quantification (BLOQ) values were plotted as zeros.

Expression of phenotypic T cell markers CD3, CD8, CD4, FOXP3 and proliferation marker Ki67 were evaluated in FFPE section by fluorescence-immunohistochemistry in a small subset of cases. Expression of OX40L, OX40 and other markers of immune response are also being evaluated by such methods.

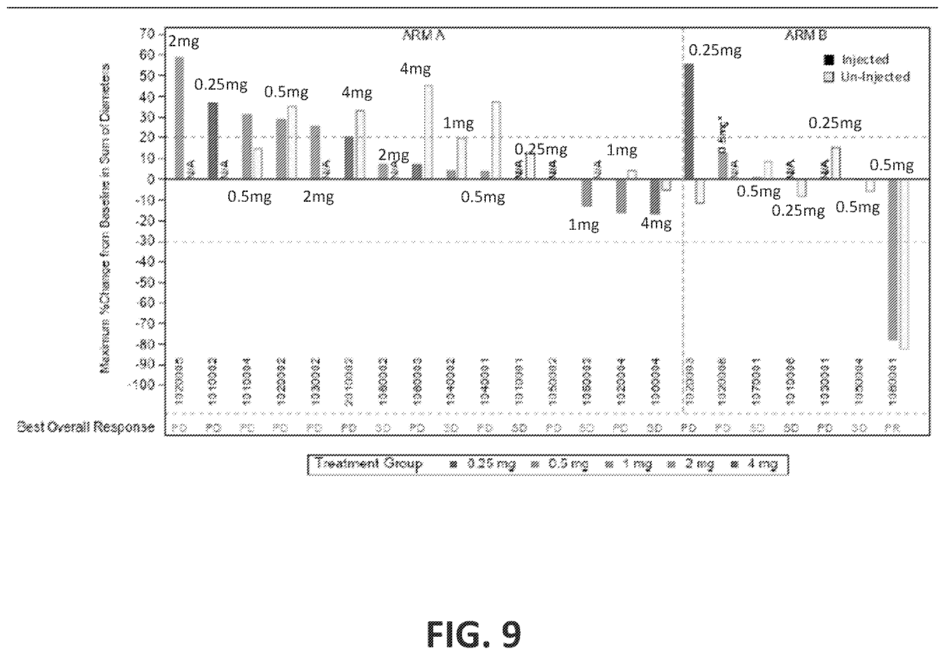

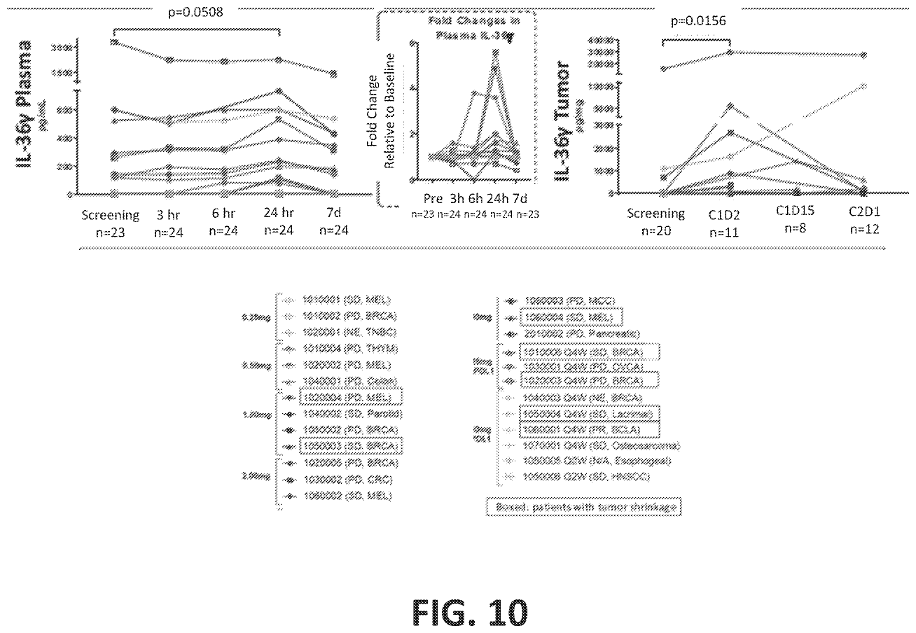

In the 23 patients evaluated, elevated IL-36γ and IL-23 protein expression was detected in tumor and plasma, with greatest increases at 24 hours post-dose. Increased IL-36γ expression was observed in tumor at all doses tested, and minor elevations were observed in plasma (7/10 and 10/23 evaluable patients, respectively; FIG. 10). Increased IL-23 expression was observed in tumor at 0.5 mg (1/3 patients), 1.0 mg (3/3 patients), 2.0 mg (1/2 patients), and 4.0 mg (1/3 patients) but not at the 0.25 mg dose level (1 evaluable patient), and expression in plasma was observed at all dose levels tested, in 18/23 evaluable patients (FIG. 11). Greatest IL-23 levels were measured from samples at the highest dose levels (2 mg and 4 mg) tested thus far, although other dose-dependent relationships in IL-23 and IL-36γ expression were not yet evident in the current dataset.

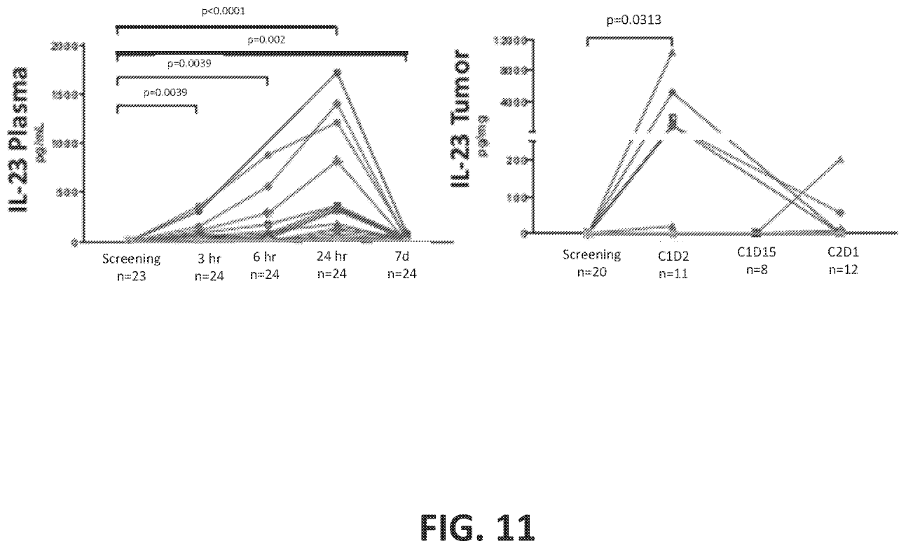

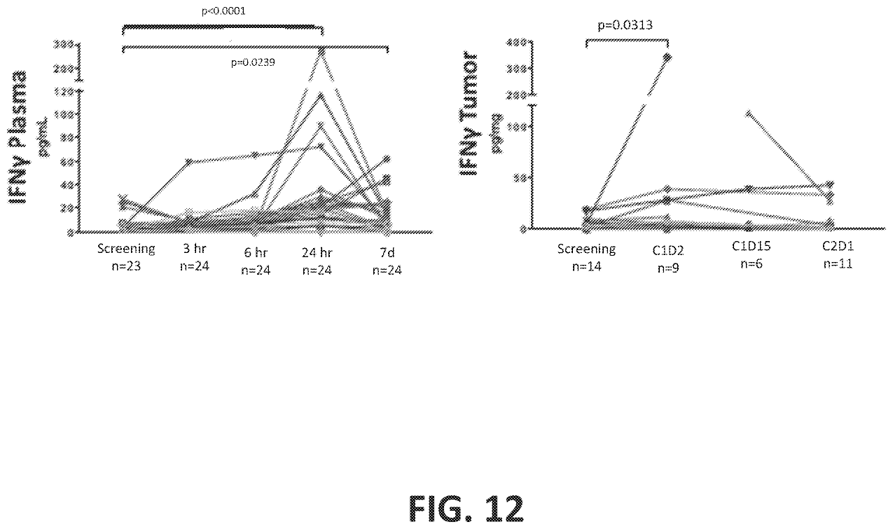

Multiple pro-inflammatory cytokines were significantly elevated 24 hours post-treatment. Key among these were IFN-γ and TNF-α in plasma and in tumor (FIGS. 12 and 13). IFN-γ is the only member of the type II interferon (IFN-II) class, is produced by activated lymphocytes, and elevated circulating levels post-treatment have been associated with improved checkpoint inhibitor response (e.g., in NSCLC, Boutsikou et al. Ther Adv Med Oncol, 2018). TNF-α in a key cytokine expressed during acute phase immune reactions and mediated of anti-tumor activity (Josephs et al. J Transl Med, 2018).

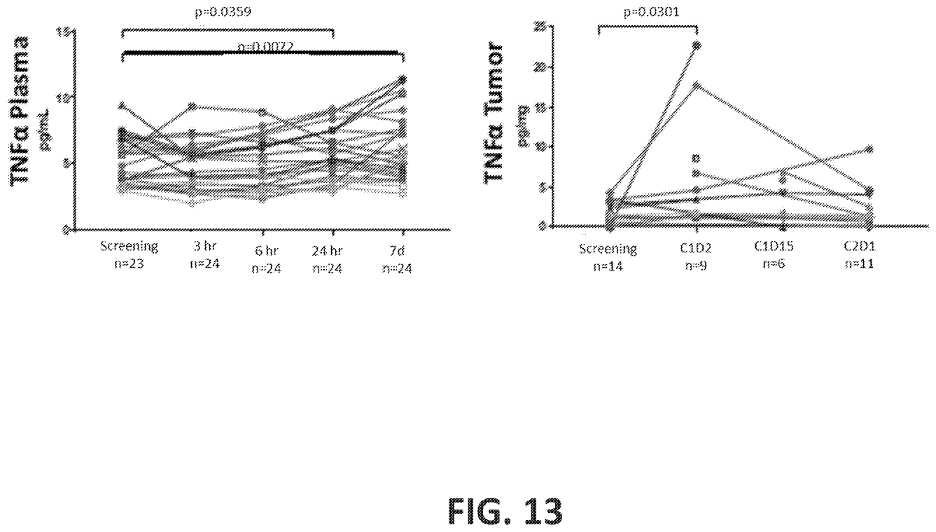

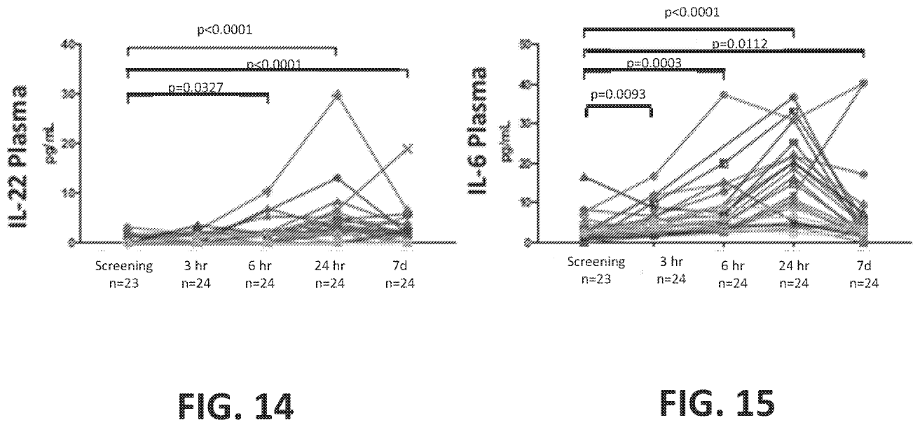

Significant increases in IL-22 and IL-6 were also observed, peaking at 24 hours post-mRNA-TRIPLET treatment in plasma (FIGS. 14 and 15). IL-22 and IL-6 are known to be induced downstream of IL-23 and IL-36γ, respectively (Hewitt et al., Sci Transl Med, 2019), with small but significant correlations in expression detected after mRNA-TRIPLET treatment. Fold changes in IL-6 expression were calculated due to variability at baseline, and the highest magnitude of changes were seen in the 4 mg and 1 mg dose levels, with all 3 assessed patients at 4 mg dosing being among the highest fold changes.

Significant increases post-treatment were also observed in plasma levels of MIP-3a, IL-27, IL-10 and IL-8 (data not shown). Cytokines typically implicated in cytokine release syndrome (IL-6, IFN-γ, TNF-α, IL-8, IL-2, IL-10) were in the low pg/mL range at the dose levels evaluated, and well below what has been suggested as clinically toxic levels for these cytokines, typically in the ng/mL range (Yiu et al. PLosOne, 2012; Hay et al. Blood, 2018).

### PD-L1 Expression Levels

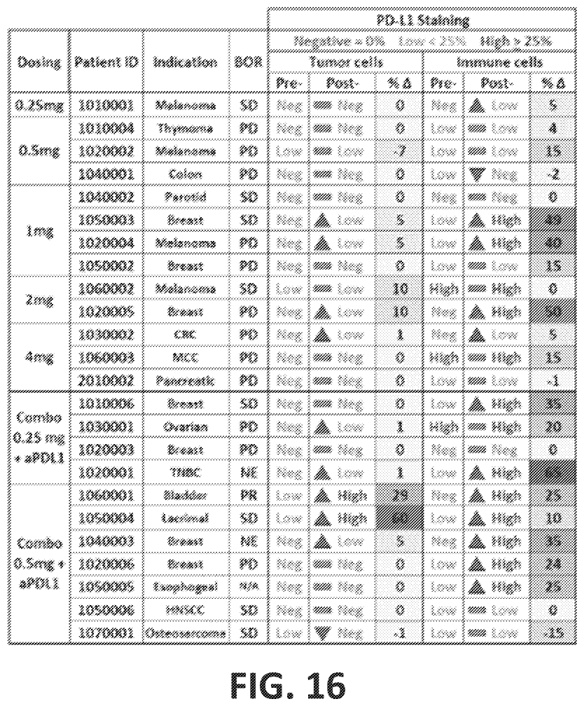

Expression of PD-L1 in tumor cells and immune cells was also evaluated. Increased PD-L1 positivity on a %-cell bases post-treatment was observed in 18/24 evaluable patients: 9 patients shifted from PD-L1 negative to positive, 3 of which shifted to PD-L1 high (>/=25% positive, Tumor Cells (TCs) and/or Immune Cells (ICs)); 8 patients shifted from PD-L1 low (<25% positive, TCs and/or ICs) to PD-L1 high; 4 patients shifted from PD-L1 low to elevated levels that fell below 25% positive; and 2 patients shifted from PD-L1 high to further elevated levels (70% to 90%, ICs) (FIG. 16). Cutoffs of low (<25%) versus high (>/=25%) PD-L1 positivity were based on reported PD-L1 levels and durvalumab response in urothelial bladder cancer, with high PD-L1 levels most predictive of response.

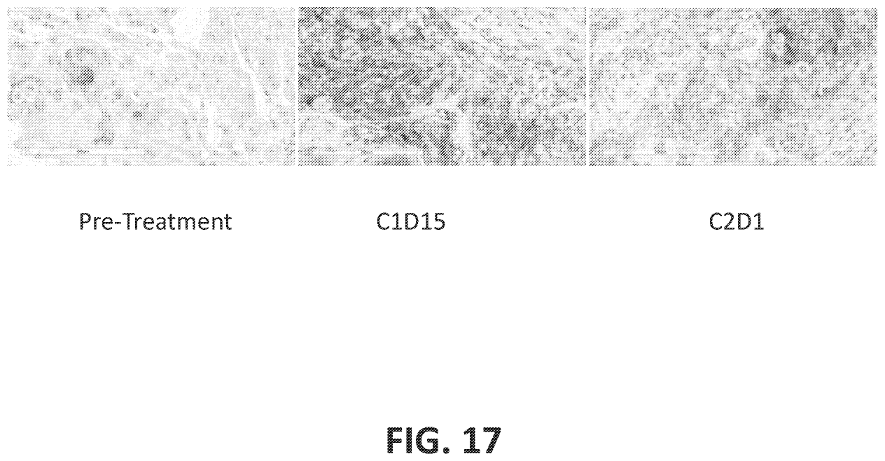

The average peak increase in PD-L1 levels post-treatment was 20.8%. The squamous-cell bladder cancer patient, for example, was 1% (TC score) and 0% (IC score) positive at baseline but shifted to 30% (TC score) and 25% (IC score) positive at cycle 1 day 15. Representative immunohistochemistry images are shown in FIG. 17. Cutoffs of low (<25%) versus high (>/=25%) PD-L1 positivity are based on reported PD-L1 levels and durvalumab response in urothelial bladder cancer, with high PD-L1 levels most predictive of response. Infiltration of proliferating T cells, particularly CD8+ T cells, into the TME was also observed in this patient, up to a month after treatment (data not shown).

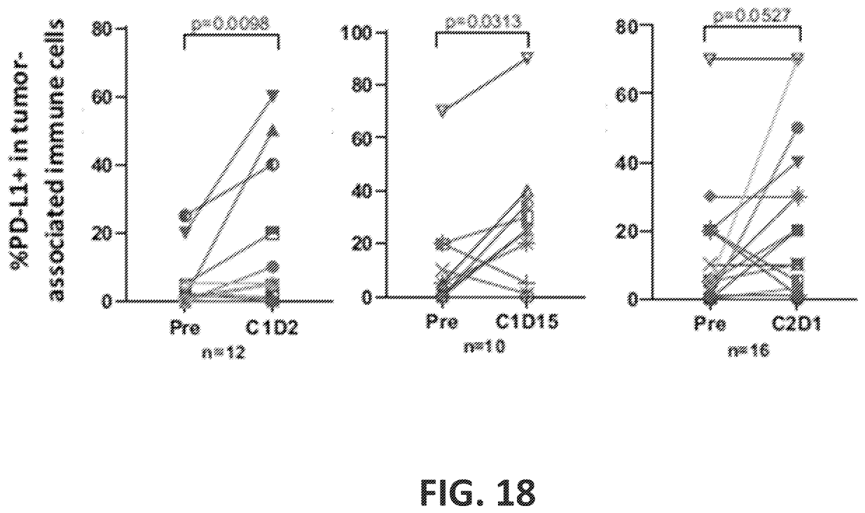

Increased PD-L1 was primarily observed in ICs in the TME (FIG. 18). More modest but significant increases in TC PD-L1 positivity were also observed. PD-L1 was most significantly increased at 1 day post-1^{st} dose (in ICs, CD1D2 for monotherapy cases and C1D15 for combo cases), though elevated PD-L1 levels persisted to cycle 2 day 1 (pre-dose), trending towards significance. Overall, PD-L1 expression was significantly higher post-treatment in both tumor and in immune cells when considering values for the highest expression level achieved at any time-point after baseline. IC positivity, aside from TC positivity, independently predicts favorable patient prognosis and correlated with durable clinical responses to a-PD-L1 (in HNSCC: Kim et al. Sci Rep, 2016; in NSCLC: Kowanetz et al. PNAS 2018). Expression of IFN-γ, a cytokine known to induce PD-L1 expression (Liang et al. Eur J Immunol, 2003, Mandai, et al. Clin Canc Res, 2016) in the tumor was correlated with PD-L1 levels (in immune cells) at baseline and, even more significantly, a week after treatment (data not shown).

### Example 7: Additional Clinical Study Design to Evaluate Anti-Tumor Efficacy of mRNAs Encoding OX40L, IL-23 and IL-36γ Alone or in Combination with Immune Checkpoint Blockade

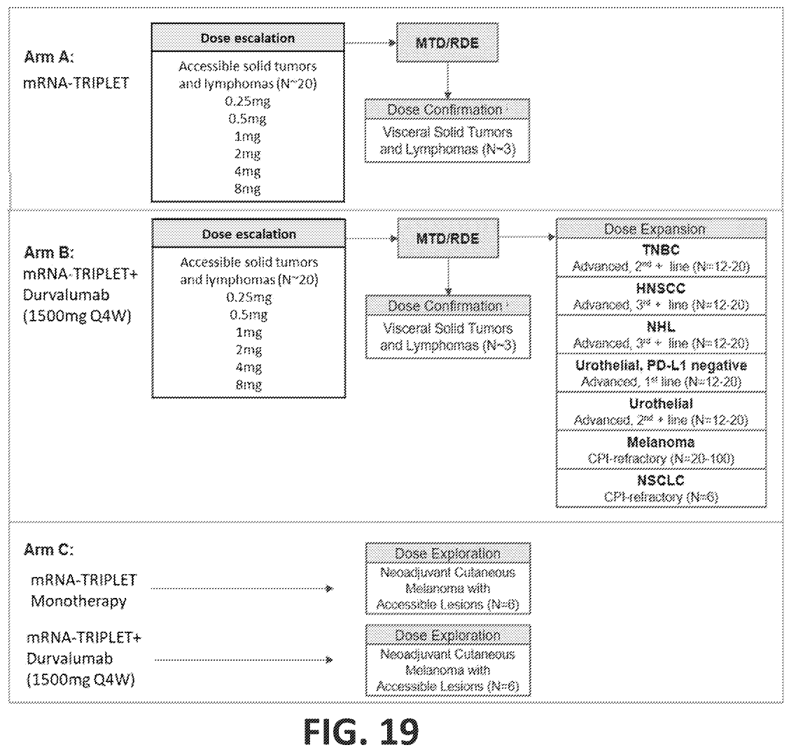

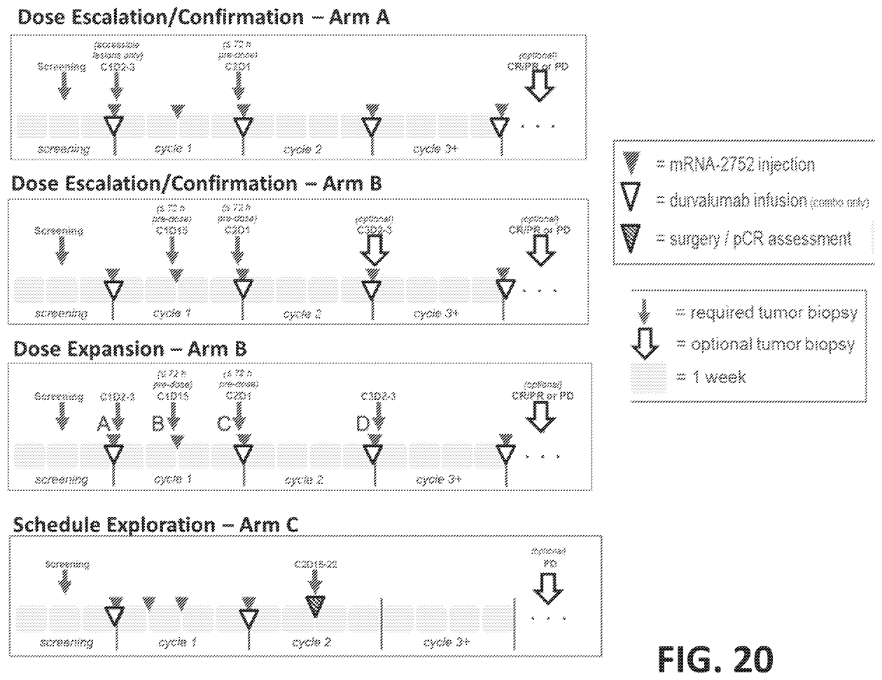

FIG. 19 is the study design of an ongoing Phase 1, dose escalation study of ITu injections of mRNA-TRIPLET comprising mRNAs encoding polypeptides of OX40L, IL-23 and IL-36γ alone and in combination with intravenously administered immune checkpoint blockade therapy (i.e., durvalumab (PD-L1 inhibitor) in patients with histologically confirmed advanced or metastatic solid tumor malignancies or lymphomas. The study includes the following three treatment arms:
Arm A: mRNA-TRIPLET alone, administered every two weeks (Q2W) for Cycle 1 and every 4 weeks (Q4W) for Cycles 2-6.
Arm B: mRNA-TRIPLET every two weeks (Q2W) for Cycle 1 and every 4 weeks (Q4W) for Cycles 2-6 in combination with durvalumab Q4W for 6 cycles. Following completion of 6 of mRNA-TRIPLET, patients may continue with durvalumab as a single agent until disease progression, unacceptable toxicity, or 24 months of treatment (total), whichever is sooner.
Arm C: mRNA-TRIPLET on Days 1, 8 and 15 for cycle 1, and day for Cycle 2 (C2D1) alone or in combination with durvalumab Q4W for 2 cycles.

The study includes 2 dose escalation and dose confirmation parts (Arms A and B) followed by a dose expansion part in select indications for Arm B, along with a dose exploration part in Arm C for neoadjuvant cutaneous melanoma. The dose expansion for Arm B is for the following indications:
Group 1: Triple-negative breast cancer (TNBC)
Group 2: Head and neck squamous cell carcinoma (HNSCC)
Group 3: Non-Hodgkin lymphoma (NHL)
Group 4: Urothelial cancer in patients that are ineligible for platinum-containing chemotherapy and are PD-L1 negative (1 L)
Group 5: Urothelial cancer in patients having objective evidence of disease progression during or following platinum-containing chemotherapy (2 L+)
Group 6: checkpoint inhibitor (CPI)-refractory melanoma
Group 7: CPI-refractory non-small cell lung carcinoma (NSCLC)

SEQUENCE TABLE

| SEQ | |
|---|---|
| ID | |
| NO: | DESCRIPTION |
| 1 | MERVQPLEENVGNAARPRFERNKLLLVASVIQGLGLLLCFTYICLHFSALQVSHRYPRIQSIKVQFTE |
| | YKKEKGFILTSQKEDEIMKVQNNSVIINCDGFYLISLKGYFSQEVNISLHYQKDEEPLFQLKKVRSVN |
| | SLMVASLTYKDKVYLNVTTDNTSLDDFHVNGGELILIHQNPGEFCVL |
| | OX40L (TNSFR4)-Tumor necrosis factor ligand superfamily member 4 |
| | isoform 1 [Homo sapiens: NP_003317 (bold is intracellular domain, |
| | italics is transmembrane domain, and underline is extracellular |
| | domain) |
| 2 | MVSHRYPRIQSIKVQFTEYKKEKGFILTSQKEDEIMKVQNNSVIINCDGFYLISLKGYFSQEVNISLH |
| | YQKDEEPLFQLKKVRSVNSLMVASLTYKDKVYLNVTTDNTSLDDFHVNGGELILIHQNPGEFCVL |
| | OX40L (TNSFR4)-TNFSF4 isoform 2 [Homo sapiens] NP_001284491 |
| 3 | MEGEGVQPLDENLENGSRPRFKWKKTLRLVVSGIKGAGMLLCFIYVCLQLSSSPAKDPPIQRLRGAVT |
| | RCEDGQLFISSYKNEYQTMEVQNNSVVIKCDGLYIIYLKGSFFQEVKIDLHFREDHNPISIPMLNDGR |
| | RIVFTVVASLAFKDKVYLTVNAPDTLCEHLQINDGELIVVQLTPGYCAPEGSYHSTVNQVPL |
| | OX40L (TNSFR4)-TNFSF4 [Mus musculus] NP_033478 |
| 4 | AUGGAAAGGGUCCAACCCCUGGAAAGAGAAUGUGGGAAAUGCAGCCAGGCCAAGAUUCAGAGGAACAA |
| | GCUAUUGCUGGUGGCCUCUGUAAUUCAGGGACUGGGGCUGCUCCUGUGCUUCACCUACAUCUGCCUGC |
| | ACUUCUCUGCUCUUCAGGUAUCACAUCGGUAUCCUCGAAUUCAAAGUAUCAAAGUACAAUUUACCGAA |
| | UAUAAGAAGGAGAAAGGUUUCAUCCUCACUUCCCAAAAGGAGGAUGAAAUCAUGAAGGUGCAGAACAA |
| | CUCAGUCAUCAUCAACUGUGAUGGGUUUUAUCUCAUCUCCCUGAAGGGCUACUUCUCCCAGGAAGUCA |
| | ACAUUAGCCUUCAUUACCAGAAGGAUGAGGAGCCCCUCUUCCAACUGAAGAAGGUCAGGUCUGUCAAC |
| | UCCUUGAUGGUGGCCUCUCUGACUUACAAAGACAAAGUCUACUUGAAUGUGACCACUGACAAUACCUC |
| | CCUGGAUGACUUCCAUGUGAAUGGCGGAGAACUGAUUCUUAUCCAUCAAAAUCCUGGUGAAUUCUGUG |
| | UCCUU |
| | Human OX40L mRNA (ORF) |
| 5 | 5′^{7Me}GₚₚₚG_{2′OMe}GGAAAUAAGAGAGAAAAGAAGAGUAAGAAGAAAUAUAAGAGCCACCAUGGAAAGGGU |
| | CCAACCCCUGGAAGAGAAUGUGGGAAAUGCAGCCAGGCCAAGAUUCGAGAGGAACAAGCUAUUGCUGG |
| | UGGCCUCUGUAAUUCAGGGACUGGGGCUGCUCCUGUGCUUCACCUACAUCUGCCUGCACUUCUCUGCU |
| | CUUCAGGUAUCACAUCGGUAUCCUCGAAUUCAAAGUAUCAAAGUACAAUUUACCGAAUAUAAGAAGGA |
| | GAAAGGUUUCAUCCUCACUUCCCAAAAGGAGGAUGAAAUCAUGAAGGUGCAGAACAACUCAGUCAUCA |
| | UCAACUGUGAUGGGUUUUAUCUCAUCUCCCUGAAGGGCUACUUCUCCCAGGAAGUCAACAUUAGCCUU |
| | CAUUACCAGAAGGAUGAGGAGCCCCUCUUCCAACUGAAGAAGGUCAGGUCUGUCAACUCCUUGAUGGU |
| | GGCCUCUCUGACUUACAAAGACAAAGUCUACUUGAAUGUGACCACUGACAAUACCUCCCUGGAUGACU |
| | UCCAUGUGAAUGGCGGAGAACUGAUUCUUAUCCAUCAAAAUCCUGGUGAAUUCUGUGUCCUUUGAUAA |
| | UAGGCUGGAGCCUCGGUGGCCAUGCUUCUUGCCCCUUGGGCCUCCCCCCAGCCCCUCCUCCCCUUCCU |
| | GCACCCGUACCCCCCAAACACCAUUGUCACACUCCAGUGGUCUUUGAAUAAAGUCUGAGUGGGCGGCA |
| | AAAAAAAAAAAAAAAAAAAAAAAAAAAAAAAAAAAAAAAAAAAAAAAAAAAAAAAAAAAAAAAAAAAA |
| | AAAAAAAAAAAAAAAAAAAAAAAAAAAAAAAUCUAG_{OH}3′ |
| | Where: A, C G & U = AMP, CMP, GMP & N1-ΨUMP, respectively; Me-methyl; |
| | p = inorganic phosphate |
| | Full-length mRNA Nucleotide sequence (5′ UTR, ORF, 3′ UTR, polyA tail) |
| | of human OX40L |
| 6 | GGCCCUGGGACCUUUGCCUAUUUUCUGAUUGAUAGGCUUUGUUUUGUCUUUACCUCCUUCUUUCUGGG |
| | GAAAACUUCAGUUUUAUCGCACGUUCCCCUUUUCCAUAUCUUCAUCUUCCCUCUACCCAGAUUGUGAA |
| | GAUGGAAAGGGUCCAACCCCUGGAAGAGAAUGUGGGAAAUGCAGCCAGGCCAAGAUUCGAGAGGAACA |
| | AGCUAUUGCUGGUGGCCUCUGUAAUUCAGGGACUGGGGCUGCUCCUGUUCUUCACCUACAUCUGCCUG |
| | CACUUCUCUGCUCUUCAGGUAUCACAUCGGUAUCCUCGAAUUCAAAGUAUCAAAGUACAAUUUACCGA |
| | AUAUAAGAAGGAGAAAGGUUUCAUCCUCACUUCCCAAAAGGAGGAUGAAAUCAUGAAGGUGCAGAACA |
| | ACUCAGUCAUCAUCAACUGUGAUGGGUUUUAUCUCAUCUCCCUGAAGGGCUACUUCUCCCAGGAAGUC |
| | AACAUUAGCCUUCAUUACCAGAAGGAUGAGGAGCCCCUCUUCCAACUGAAGAAGGUCAGGUCUGUCAA |
| | CUCCUUGAUGGUGGCCUCUCUGACUUACAAAGACAAAGUCUACUUGAAUGUGACCACUGACAAUACCU |
| | CCCUGGAUGACUUCCAUGUGAAUGGCGGAGAACUGAUUCUUAUCCAUCAAAAUCCUGGUGAAUUCUGU |
| | GUCCUUUGAGGGGCUGAUGGCAAUAUCUAAAACCAGGCACCAGCAUGAACACCAAGCUGGGGGUGGAC |
| | AGGGCAUGGAUUCUUCAUUGCAAGUGAAGGAGCCUCCCAGCUCAGCCACGUGGGAUGUGACAAGAAGC |
| | AGAUCCUGGCCCUCCCGCCCCCACCCCUCAGGGAUAUUUAAAACUUAUUUUAUAUACCAGUUAAUCUU |
| | AUUUAUCCUUAUAUUUUCUAAAUUGCCUAGCCGUCACACCCCAAGAUUGCCUUGAGCCUACUAGGCAC |
| | CUUUGUGAGAAAGAAAAAAUAGAUGCCUCUUCUUCAAGAUGCAUUGUUUCUAUUGGUCAGGCAAUUGU |
| | CAUAAUAAACUUAUGUCAUUGAAAACGGUACCUGACUACCAUUUGCUGGAAAUUUGACAUGUGUGUGG |
| | CAUUAUCAAAAUGAAGAGGAGCAAGGAGUGAAGGAGUGGGGUUAUGAAUCUGCCAAAGGUGGUAUGAA |
| | CCAACCCCUGGAAGCCAAAGCGGCCUCUCCAAGGUUAAAUUGAUUGCAGUUUGCAUAUUGCCUAAAUU |
| | UAAACUUUCUCAUUUGGUGGGGGUUCAAAAGAAGAAUCAGCUUGUGAAAAAUCAGGACUUGAAGAGAG |
| | CCGUCUAAGAAAUACCACGUGCUUUUUUUCUUUACCAUUUUGCUUUCCCAGCCUCCAAACAUAGUUAA |
| | UAGAAAUUUCCCUUCAAAGAACUGUCUGGGGAUGUGAUGCUUUGAAAAAUCUAAUCAGUGACUUAAGA |
| | GAGAUUUUCUUGUAUACAGGGAGAGUGAGAUAACUUAUUGUGAAGGGUUAGCUUUACUGUACAGGAUA |
| | GCAGGGAACUGGACAUCUCAGGGUAAAAGUCAGUACGGAUUUUAAUAGCCUGGGGAGGAAAACACAUU |
| | CUUUGCCACAGACAGGCAAAGCAACACAUGCUCAUCCUCCUGCCUAUGCUGAGAUACGCACUCAGCUC |
| | CAUGUCUUGUACACACAGAAACAUUGCUGGUUUCAAGAAAUGAGGUGAUCCUAUUAUCAAAUUCAAUC |
| | UGAUGUCAAAUAGCACUAAGAAGUUAUUGUGCCUUAUGAAAAAUAAUGAUCUCUGUCUAGAAAUACCA |
| | UAGACCAUAUAUAGUCUCACAUUGAUAAUUGAAACUAGAAGGGUCUAUAAUCAGCCUAUGCCAGGGCU |
| | UCAAUGGAAUAGUAUCCCCUUAUGUUUAGUUGAAAUGUCCCCUUAACUUGAUAUAAUGUGUUAUGCUU |
| | AUGGCGCUGUGGACAAUCUGAUUUUUCAUGUCAACUUUCCAGAUGAUUUGUAACUUCUCUGUGCCAAA |
| | CCUUUUAUAAACAUAAAUUUUUGAGAUAUGUAUUUUAAAAUUGUAGCACAUGUUUCCCUGACAUUUUC |
| | AAUAGAGGAUACAACAUCACAGAAUCUUUCUGGAUGAUUCUGUGUUAUCAAGGAAUUGUACUGUGCUA |
| | CAAUUAUCUCUAGAAUCUCCAGAAAGGUGGAGGGCUGUUCGCCCUUACACUAAAUGGUCUCAGUUGGA |
| | UUUUUUUUUCCUGUUUUCUAUUUCCUCUUAAGUACACCUUCAACUAUAUUCCCAUCCCUCUAUUUUAA |
| | UCUGUUAUGAAGGAAGGUAAAUAAAAAUGCUAAAUAGAAGAAAUUGUAGGUAAGGUAAGAGGAAUCAA |
| | GUUCUGAGUGGCUGCCAAGGCACUCACAGAAUCAUAAUCAUGGCUAAAUAUUUAUGGAGGGCCUACUG |
| | UGGACCAGGCACUGGGCUAAAUACUUACAUUUACAAGAAUCAUUCUGAGACAGAUAUUCAAUGAUAUC |
| | UGGCUUCACUACUCAGAAGAUUGUGUGUGUGUUUGUGUGUGUGUGUGUGUGUGUAUUUCACUUUUUGU |
| | UAUUGACCAUGUUCUGCAAAAUUGCAGUUACUCAGUGAGUGAUAUCCGAAAAAGUAAACGUUUAUGAC |
| | UAUAGGUAAUAUUUAAGAAAAUGCAUGGUUCAUUUUUAAGUUUGGAAUUUUUAUCUAUAUUUCUCACA |
| | GAUGUGCAGUGCACAUGCAGGCCUAAGUAUAUGUUGUGUGUGUUGUUUGUCUUUGAUGUCAUGGUCCC |
| | CUCUCUUAGGUGCUCACUCGCUUUGGGUGCACCUGGCCUGCUCUUCCCAUGUUGGCCUCUGCAACCAC |
| | ACAGGGAUAUUUCUGCUAUGCACCAGCCUCACUCCACCUUCCUUCCAUCAAAAAUAUGUGUGUGUGUC |
| | UCAGUCCCUGUAAGUCAUGUCCUUCACAGGGAGAAUUAACCCUUCGAUAUACAUGGCAGAGUUUUGUG |
| | GGAAAAGAAUUGAAUGAAAAGUCAGGAGAUCAGAAUUUUAAAUUUGACUUAGCCACUAACUAGCCAUG |
| | UAACCUUGGGAAAGUCAUUUCCCAUUUCUGGGUCUUGCUUUUCUUUCUGUUAAAUGAGAGGAAUGUUA |
| | AAUAUCUAACAGUUUAGAAUCUUAUGCUUACAGUGUUAUCUGUGAAUGCACAUAUUAAAUGUCUAUGU |
| | UCUUGUUGCUAUGAGUCAAGGAGUGUAACCUUCUCCUUUACUAUGUUGAAUGUAUUUUUUUCUGGACA |
| | AGCUUACAUCUUCCUCAGCCAUCUUUGUGAGUCCUUCAAGAGCAGUUAUCAAUUGUUAGUUAGAUAUU |
| | UUCUAUUUAGAGAAUGCUUAAGGGAUUCCAAUCCCGAUCCAAAUCAUAAUUUGUUCUUAAGUAUACUG |
| | GGCAGGUCCCCUAUUUUAAGUCAUAAUUUUGUAUUUAGUGCUUUCCUGGCUCUCAGAGAGUAUUAAUA |
| | UUGAUAUUAAUAAUAUAGUUAAUAGUAAUAUUGCUAUUUACAUGGAAACAAAUAAAAGAUCUCAGAAU |
| | UCACUAAAAAAAAAAA |
| | OX40L-TNFSF4, transcript variant 1, mRNA NM_003326 |
| 7 | GGGAAAUAAGAGAGAAAAGAAGAGUAAGAAGAAAUAUAAGAGCCACCAUGGAAAGGGUCCAACCCCUG |
| | GAAGAGAAUGUGGGAAAUGCAGCCAGGCCAAGAUUCGAGAGGAACAAGCUAUUGCUGGUGGCCUCUGU |
| | AAUUCAGGGACUGGGGCUGCUCCUGUGCUUCACCUACAUCUGCCUGCACUUCUCUGCUCUUCAGGUAU |
| | CACAUCGGUAUCCUCGAAUUCAAAGUAUCAAAGUACAAUUUACCGAAUAUAAGAAGGAGAAAGGUUUC |
| | AUCCUCACUUCCCAAAAGGAGGAUGAAAUCAUGAAGGUGCAGAACAACUCAGUCAUCAUCAACUGUGA |
| | UGGGUUUUAUCUCAUCUCCCUGAAGGGCUACUUCUCCCAGGAAGUCAACAUUAGCCUUCAUUACCAGA |
| | AGGAUGAGGAGCCCCUCUUCCAACUGAAGAAGGUCAGGUCUGUCAACUCCUUGAUGGUGGCCUCUCUG |
| | ACUUACAAAGACAAAGUCUACUUGAAUGUGACCACUGACAAUACCUCCCUGGAUGACUUCGAUGUGAA |
| | UGGCGGAGAACUGAUUCUUAUCCAUCAAAAUCCUGGUGAAUUCUGUGUCCUUUGAUAAUAGGCUGGAG |
| | CCUCGGUGGCCAUGCUUCUUGCCCCUUGGGCCUCCCCCCAGCCCCUCCUCCCCUUCCUGCACCCGUAC |
| | CCCCCAAACACCAUUGUCACACUCCAGUGGUCUUUGAAUAAAGUCUGAGUGGGCGGC |
| | mRNA sequence: Human OX40L with 5′-UTR, 3′-UTR, and miR-122 binding |
| | site |
| 8 | AUGGAGCGGGUGCAGCCCCUGGAGGAGAACGUGGGCAACGCCGCUCGGCCACGGUUCGAGCGGAACAA |
| | GCUGCUGCUGGUGGCUAGCGUGAUCCAGGGCCUGGGCCUGCUGCUGUGCUUCACCUACAUCUGCCUGC |
| | ACUUCAGCGCCCUGCAGGUGAGCCACCGGUAUCCCCGGAUCCAGAGCAUCAAGGUGCAGUUCACCGAG |
| | UACAAGAAGGAGAAGGGCUUCAUCCUGACCAGCCAGAAGGAGGACGAGAUCAUGAAGGUGCAGAACAA |
| | CAGCGUGAUCAUCAACUGCGACGGCUUCUACCUGAUCAGCCUGAAGGGCUACUUCAGCCAGGAGGUGA |
| | ACAUCAGCCUGCACUACCAGAAGGACGAGGAGCCCCUGUUCCAGCUGAAGAAGGUGCGGAGCGUGAAC |
| | AGCCUGAUGGUGGCCAGCCUGACCUACAAGGACAAGGUGUACCUGAACGUGACCACCGACAACACCAG |
| | CCUGGACGACUUCCACGUGAACGGCGGCGAGCUGAUCCUGAUCCACCAGAACCCCGGCGAGUUCUGCG |
| | UGCUG |
| | mRNA open reading frame sequence 1 for Human OX40L |
| 9 | AUAGAAAGGGUCCAACCCCUCGAAGAGAACGUGGGAAACGCAGCCAGGCCAAGAUUCGAGAGGAACAA |
| | GCUAUUGCUCGUGGCCUCAGUAAUUCAGGGACUCGGGUUACUCCUUUGCUUCACCUACAUCUGCUUGC |
| | ACUUCAGUGCUCUGCAGGUAUCACAUCGGUAUCCUCGAAUUCAAAGUAUCAAAGUACAAUUUACCGAA |
| | UAUAAGAAGGAGAAAGGUUUCAUCCUCACUUCCCAGAAGGAGGAUGAAAUCAUGAAGGUGCAGAACAA |
| | CUCAGUCAUCAUCAACUGUGAUGGGUUUUAUCUCAUCUCCCUGAAGGGCUACUUCUCCCAGGAAGUCA |
| | ACAUUAGCCUUCAUUACCAGAAGGAUGAGGAGCCCCUCUUCCAACUGAAGAAGGUCAGGUCUGUCAAC |
| | UCCUUGAUGGUAGCCUCUCUGACUUACAAAGACAAAGUCUACUUGAAUGUGACCACUGACAAUACCUC |
| | CCUGGAUGACUUCCAUGUGAAUGGCGGAGAACUGAUUCUUAUCCAUCAGAAUCCUGGUGAAUUCUGUG |
| | UCCUU |
| | mRNA open reading frame sequence 2 for Human OX40L |
| 10 | AUGGAGCGGGUGCAGCCCCUGGAGGAGAACGUGGGCAACGCCGCUCGGCCACGGUUCGAGCGGAACAA |
| | GCUGCUGCUGGUGGCUAGCGUGAUCCAGGGCCUGGGCCUGCUGCUGUGCUUCACCUACAUCUGCCUGC |
| | ACUUCAGCGCCCUGCAGGUGAGCCACCGGUAUCCCCGGAUCCAGAGCAUCAAGGUGCAGUUCACCGAG |
| | UACAAGAAGGAGAAGGGCUUCAUCCUGACCAGCCAGAAGGAGGACGAGAUCAUGAAGGUGCAGAACAA |
| | CAGCGUGAUCAUCAACUGCGACGGCUUCUACCUGAUCAGCCUGAAGGGCUACUUCAGCCAGGAGGUGA |
| | ACAUCAGCCUGCACUACCAGAAGGACGAGGAGCCCCUGUUCCAGCUGAAGAAGGUGCGGAGCGUGAAC |
| | AGCCUGAUGGUGGCCAGCCUGACCUACAAGGACAAGGUGUACCUGAACGUGACCACCGACAACACCAG |
| | CCUGGACGACUUCCACGUGAACGGCGGCGAGCUGAUCCUGAUCCACCAGAACCCCGGCGAGUUCUGCG |
| | UGCUG |
| | mRNA open reading frame sequence 3 for Human OX40L |
| 11 | AGGAAAUAAGAGAGAAAAGAAGAGUAAGAAGAAAUAUAAGACCCCGGCGCCGCCACC |
| | 5′ UTR |
| 12 | CCUUAGCAGAGCUGUGGAGUGUGACAAUGGUGUUUGUGUCUAAACUAUCAAACGCCAUUAUCACACUA |
| | AAUAGCUACUGCUAGGC |
| | (miR-122) |
| 13 | AACGCCAUUAUCACACUAAAUA |
| | (miR-122-3p) |
| 14 | UAUUUAGUGUGAUAAUGGCGUU (miR-122-3p binding site) |
| 15 | UCAAGCUUUUGGACCCUCGUACAGAAGCUAAUACGACUCACUAUAGGGAAAUAAGAGAGAAAAGAAGA |
| | GUAAGAAGAAAUAUAAGAGCCACC |
| | (5′ UTR) |
| 16 | GGGAAAUAAGAGAGAAAAGAAGAGUAAGAAGAAAUAUAAGAGCCACC |
| | (5′ UTR) |
| 17 | UGAUAAUAGGCUGGAGCCUCGGUUGCCAUGCUUCUUGCCCCUUGGGCCUCCCCCCAGCCCCUCCUCCC |
| | CUUCCUGCACCCGUACCCCCCAAACACCAUUGUCACACUCCAGUGGUCUUUGAAUAAAGUCUGAGUGG |
| | GCGGC (3′ UTR) |
| 18 | UGAUAAUAGGCUGGAGCCUCGGUGGCCAUGCUUCUUGCCCCUUGGGCCCAAACACCAUUGUCACACUC |
| | CAUCCCCCCAGCCCCUCCUCCCCUUCCUCCAUAAAGUAGGAAACACUACAUGCACCCGUACCCCCGUG |
| | GUCUUUGAAUAAAGUCUGAGUGGGCGGC |
| | (3′ UTR with mi-122 and mi-142.3p sites) |
| 19 | UGGAGUGUGACAAUGGUGUUUG |
| | (miR-122-5p) |
| 20 | CAAACACCAUUGUCACACUCCA (miR-122-5p binding site) |
| 21 | GCCA/GCC |
| | Kozak Consensus |
| 22 | UGAUAAUAGGCUGGAGCCUCGGUGGCCUAGCUUCUUGCCCCUUGGGCCUCCCCCCAGCCCCUCCUCCC |
| | CUUCCUGCACCCGUACCCCCCAAACACCAUUGUCACACUCCAGUGGUCUUUGAAUAAAGUCUGAGUGG |
| | GCGGC |
| | 3′ UTR with miR-122 |
| 23 | GGGAAATAAGAGAGAAAAGAAGAGTAAGAAGAAATATAAGAGCCACC |
| | Standard UTR |
| 24 | MCHQQLVISWFSLVFLASPLVAIWELKKDVYVVELDWYPDAPGEMVVLTCDTPEEDGITWTLDQSSEV |
| | LGSGKTLTIQVKEFGDAGQYTCHKGGEVLSHSLLLLHKKEDGIWSTDILKDQKEPKNKTFLRCEAKNY |
| | SGRFTCWWLTTISTDLTFSVKSSRGSSDPQGVTCGAATLSAERVRGDNKEYEYSVECQEDSACPAAEE |
| | SLPIEVMVDAVHKLKYENYTSSFFIRDIIKPDPPKNLQLKPLKNSRQVEVSWEYPDTWSTPHSYFSLT |
| | FCVQVQGKSKREKKDRVFTDKTSATVICRKNASISVRAQDRYYSSSWSEWASVPCSGGGGGGSRAVPG |
| | GSSPAWTQCQQLSQKLCTLAWSAHPLVGHMDLREEGDEETTNDVPHIQCGDGCDPQGLRDNSQFCLQR |
| | IHQGLIFYEKLLGSDIFTGEPSLLPDSPVGQLHASLLGLSQLLQPEGHHWETQQIPSLSPSQPWQRLL |
| | LRFKILRSLQAFVAVAARVFAHGAATLSP |
| | Amino Acid sequence of human IL-23 (IL-12p40 subunit and IL-23p19 |
| | subunit linked by GS Linker) |
| 25 | AUGUGUCACCAGCAGUUGGUCAUCUCUUGGUUUUCCCUGGUAUUUCUGGCAUCUCCCCUCGUGGCCAU |
| | AUGGGAACUGAAGAAAGAUGUUUAUGUCGUAGAAUUGGAUUGGUAUCCGGAUGCCCCUGGAGAAAUGG |
| | UGGUCCUCACCUGUGACACCCCUGAAGAAGAUGGUAUCACCUGGACCUUGGACCAGAGCAGUGAGGUC |
| | UUAGGCUCUGGCAAGACCCUGACCAUCCAAGUCAAAGAGUUUGGAGAUGCUGGCCAGUACACCUGUCA |
| | CAAAGGAGGCGAGGUUCUAAGCCAUUCGCUCCUGCUGCUUCACAAGAAGGAAGAUGGAAUUUGGUCCA |
| | CUGAUAUUUUAAAGGACCAGAAAGAACCCAAGAAUAAGACCUUUCUAAGAUGCGAGGCCAAGAAUUAU |
| | UCUGGACGUUUCACCUGCUGGUGGCUGACGACAAUCAGUACUGAUUUGACAUUCAGUGUCAAGAGCAG |
| | CAGAGGCUCUUCUGACCCCCAAGGGGUGACGUGCGGAGCUGCUACACUCUCUGCAGAGAGAGUCAGAG |
| | GGGACAACAAGGAGUAUGAGUACUCAGUGGAGUGCCAGGAGGACAGUGCCUGCCCAGCUGCUGAGGAG |
| | AGUCUGCCCAUUGAGGUCAUGGUGGAUGCCGUUCACAAGCUCAAGUAUGAGAACUACACCAGCAGCUU |
| | CUUCAUCAGGGACAUCAUCAAACCUGACCCACCCAAGAACUUGCAGCUGAAGCCAUUAAAGAAUUCUC |
| | GGCAGGUGGAGGUCAGCUGGGAGUACCCUGACACCUGGAGUACUCCACAUUCCUACUUCUCCCUGACA |
| | UUCUGCGUUCAGGUCCAGGGCAAGAGCAAGAGAGAGAAGAAAGAUAGAGUCUUCACGGACAAGACCUC |
| | AGCCACGGUCAUCUGCCGCAAGAAUGCCAGCAUUAGCGUGCGGGCCCAGGACCGCUACUAUAGCUCAU |
| | CUUGGAGCGAAUGGUCAUCUGUGCCCUGCAGUGGCGGAGGGGGCGGAGGGAGCAGAGCUGUGCCUGGG |
| | GGCAGCAGCCCUGCCUGGACUCAGUGCCAGCAGCUUUCACAGAAGCUCUGCACACUGGCCUGGAGUGC |
| | ACAUCCACUAGUGGGACACAUGGAUCUAAGAGAAGAGGGAGAUGAAGAGACUACAAAUGAUGUUCCCC |
| | AUAUCCAGUGUGGAGAUGGCUGUGACCCCCAAGGACUCAGGGACAACAGUCAGUUCUGCUUGCAAAGG |
| | AUCCACCAGGGUCUGAUCUUUUAUGAGAAGCUGCUAGGAUCGGAUAUUUUCACAGGGGAGCCUUCUCU |
| | GCUCCCUGAUAGCCCUGUGGGCCAGCUUCAUGCCUCCCUACUGGGCCUCAGCCAACUCCUGCAGCCUG |
| | AGGGUCACCACUGGGAGACUCAGCAGAUUCCAAGCCUCAGUCCCAGCCAGCCAUGGCAGCGUCUCCUU |
| | CUCCGCUUCAAGAUCCUUCGCAGCCUCCAGGCCUUUGUGGCUGUAGCCGCCCGGGUCUUUGCCCAUGG |
| | AGCAGCAACCCUGAGUCCC |
| | Nucleotide sequence (ORF) of human IL-23 (IL-12p40 subunit and IL- |
| | 23p19 subunit linked by GS Linker) |
| 26 | 5′^{7Me}GₚₚₚG_{2′OMe}GGAAAUAAGAGAGAAAAGAAGAGUAAGAAGAAAUAUAAGAGCCACCAUGUGUCACCA |
| | GCAGUUGGUCAUCUCUUGGUUUUCCCUGGUAUUUCUGGCAUCUCCCCUCGUGGCCAUAUGGGAACUGA |
| | AGAAAGAUGUUUAUGUCGUAGAAUUGGAUUGGUAUCCGGAUGCCCCUGGAGAAAUGGUGGUCCUCACC |
| | UGUGACACCCCUGAAGAAGAUGGUAUCACCUGGACCUUGGACCAGAGCAGUGAGGUCUUAGGCUCUGG |
| | CAAGACCCUGACCAUCCAAGUCAAAGAGUUUGGAGAUGCUGGCCAGUACACCUGUCACAAAGGAGGCG |
| | AGGUUCUAAGCCAUUCGCUCCUGCUGCUUCACAAGAAGGAAGAUGGAAUUUGGUCCACUGAUAUUUUA |
| | AAGGACCAGAAAGAACCCAAGAAUAAGACCUUUCUAAGAUGCGAGGCCAAGAAUUAUUCUGGACGUUU |
| | CACCUGCUGGUGGCUGACGACAAUCAGUACUGAUUUGACAUUCAGUGUCAAGAGCAGCAGAGGCUCUU |
| | CUGACCCCCAAGGGGUGACGUGCGGAGCUGCUACACUCUCUGCAGAGAGAGUCAGAGGGGACAACAAG |
| | GAGUAUGAGUACUCAGUGGAGUGCCAGGAGGACAGUGCCUGCCCAGCUGCUGAGGAGAGUCUGCCCAU |
| | UGAGGUCAUGGUGGAUGCCGUUCACAAGCUCAAGUAUGAGAACUACACCAGCAGCUUCUUCAUCAGGG |
| | ACAUCAUCAAACCUGACCCACCCAAGAACUUGCAGCUGAAGCCAUUAAAGAAUUCUCGGCAGGUGGAG |
| | GUCAGCUGGGAGUACCCUGACACCUGGAGUACUCCACAUUCCUACUUCUCCCUGACAUUCUGCGUUCA |
| | GGUCCAGGGCAAGAGCAAGAGAGAGAAGAAAGAUAGAGUCUUCACGGACAAGACCUCAGCCACGGUCA |
| | UCUGCCGCAAGAAUGCCAGCAUUAGCGUGCGGGCCCAGGACCGCUACUAUAGCUCAUCUUGGAGCGAA |
| | UGGGCAUCUGUGCCCUGCAGUGGCGGAGGGGGCGGAGGGAGCAGAGCUGUGCCUGGGGGCAGCAGCCC |
| | UGCCUGGACUCAGUGCCAGCAGCUUUCACAGAAGCUCUGCACACUGGCCUGGAGUGCACAUCCACUAG |
| | UGGGACACAUGGAUCUAAGAGAAGAGGGAGAUGAAGAGACUACAAAUGAUGUUCCCCAUAUCCAGUGU |
| | GGAGAUGGCUGUGACCCCCAAGGACUCAGGGACAACAGUCAGUUCUGCUUGCAAAGGAUCCACCAGGG |
| | UCUGAUCUUUUAUGAGAAGCUGCUAGGAUCGGAUAUUUUCACAGGGGAGCCUUCUCUGCUCCCUGAUA |
| | GCCCUGUGGGCCAGCUUCAUGCCUCCCUACUGGGCCUCAGCCAACUCCUGCAGCCUGAGGGUCACCAC |
| | UGGGAGACUCAGCAGAUUCCAAGCCUCAGUCCCAGCCAGCCAUGGCAGCGUCUCCUUCUCCGCUUCAA |
| | GAUCCUUCGCAGCCUCCAGGCCUUUGUGGCUGUAGCCGCCCGGGUCUUUGCCCAUGGAGCAGCAACCC |
| | UGAGUCCCUGAUAAUAGGCUGGAGCCUCGGUGGCCAUGCUUCUUGCCCCUUGGGCCUCCCCCCAGCCC |
| | CUCCUCCCCUUCCUGCACCCGUACCCCCCAAACACCAUUGUCACACUCCAGUGGUCUUUGAAUAAAGU |
| | CUGAGUGGGCGGCAAAAAAAAAAAAAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAAAUCUAG_{OH}3′ |
| | Where: A, C G & U = AMP, CMP, GMP & N1-ΨUMP, respectively; Me = methyl; |
| | p = inorganic phosphate |
| | Full-length mRNA Nucleotide sequence (5′ UTR, ORF, 3′ UTR, mir-122- |
| | 5p (underlined) polyA tail) of human IL-23 (IL-12p40 subunit and IL- |
| | 23p19 subunit linked by GS Linker) |
| 27 | MVLQTQVFISLLLWISGAYGSMCKPITGTINDLNQQVWTLQGQNLVAVPRSDSVTPVTVAVITCKYPE |
| | ALEQGRGDPIYLGIQNPEMCLYCEKVGEQPTLQLKEQKIMDLYGQPEPVKPFLFYRAKTGRTSTLESV |
| | AFPDWFIASSKRDQPIILTSELGRSYNTAHELNLND |
| | hIGKV4-hIL-36γ construct (protein) |
| 28 | AUGGUGUUGCAGACCCAGGUCUUCAUUUCUCUGUUGCUCUGGAUCUCUGGUGCCUACGGGUCAAUGUG |
| | UAAACCUAUUACUGGGACUAUUAAUGAUUUGAAUCAGCAAGUGUGGACCCUUCAGGGUCAGAACCUUG |
| | UGGCAGUUCCACGAAGUGACAGUGUGACCCCAGUCACUGUUGCUGUUAUCACAUGCAAGUAUCCAGAG |
| | GCUCUUGAGCAAGGCAGAGGGGAUCCCAUUUAUUUGGGAAUCCAGAAUCCAGAAAUGUGUUUGUAUUG |
| | UGAGAAGGUUGGAGAACAGCCCACAUUGCAGCUAAAAGAGCAGAAGAUCAUGGAUCUGUAUGGCCAAC |
| | CCGAGCCCGUGAAAuuCUUCCUUUUCUACCGUGCCAAGACUGGUAGGACCUCCACCCUUGAGUCUGUG |
| | GCCUUCCCGGACUGGUUCAUUGCCUCCUCCAAGAGAGACCAGCCCAUCAUUCUGACUUCAGAACUUGG |
| | GAAGUCAUACAACACTGCCUUUGAAUUAAAUAUAAAUGAC |
| | Human IL-36γ mRNA (ORF) |
| 29 | 5′^{7Me}GₚₚₚG_{2′OMe}GGAAAUAAGAGAGAAAAGAAGAGUAAGAAGAAAUAUAAGAGCCACCAUGGUGUUGCA |
| | GACCCAGGUCUUCAUUUCTCUGUUGCUCUGGAUCUCUGGUGCCUACGGGUCAAUGUGUAAACCUAUUA |
| | CUGGGACUAUUAAUGAUUUGAAUCAGCAAGUGUGGACCCUUCAGGGUCAGAACCUUGUGGCAGUUCCA |
| | CGAAGUGACAGUGUGACCCCAGUCACUGUUGCUGUUAUCACAUGCAAGUAUCCAGAGGCUCUUGAGCA |
| | AGGCAGAGGGGAUCCCAUUUAUUUGGGAAUCCAGAAUCCAGAAAUGUGUUUGUAUUGUGAGAAGGUUG |
| | GAGAACAGCCCACAUUGCAGCUAAAAGAGCAGAAGAUCAUGGAUCUGUAUGGCCAACCCGAGCCCGUG |
| | AAACCCUUCCUUUUCUACCGUGCCAAGACUGGUAGGACCUCCACCCUUGAGUCUGUGGCCUUCCCGGA |
| | CUGGUUCAUUGCCUCCUCCAAGAGAGACCAGCCCAUCAUUCUGACUUCAGAACUUGGGAAGUCAUACA |
| | ACACUGCCUUUGAAUUAAAUAUAAAUGACUGAUAAUAGGCUGGAGCCUCGGUGGCCAUGCUUCUUGCC |
| | CCUUGGGCCUCCCCCCAGCCCCUCCUCCCCUUCCUGCACCCGUACCCCCCAAACACCAUUGUCACACU |
| | CCAGUGGUCUUUGAAUAAAGUCUGAGUGGGCGGCAAAAAAAAAAAAAAAAAAAAAAAAAAAAAAAAAA |
| | AAAAAAAAAAAAAAAAAAAAAAAAAAAAAAAAAAAAAAAAAAAAAAAAAAAAAAAAAAAAAAAAAAUC |
| | UAG_{OH}3′ |
| | Where: A, C G & U = AMP, CMP, GMP & N1-ΨUMP, respectively; Me = methyl; |
| | p = inorganic phosphate |
| | Full-length mRNA Nucleotide sequence (5′ UTR, ORF, 3′ UTR, mir-122- |
| | 5p (underlined) polyA tail) of human IL-36-gamma |

1. A method for treating advanced or metastatic solid tumor malignancy or lymphoma in a human patient, comprising administering to the patient by intratumoral injection an effective amount of a lipid nanoparticle (LNP) encapsulated messenger RNA (mRNA) therapeutic agent comprising three mRNA drug substances:
(i) a first mRNA comprising an open reading frame (ORF) encoding a human OX40L polypeptide;
(ii) a second mRNA comprising an ORF encoding a human IL-23 polypeptide; and
(iii) a third mRNA comprising an ORF encoding a human IL-36γ polypeptide,
wherein the patient is administered a dose of 0.10-10.0 mg of the mRNA therapeutic agent in a dosing regimen comprising a first dosing cycle and at least one subsequent dosing cycle, wherein the first and subsequent dosing cycles comprise administration of the mRNA therapeutic at a dosing interval selected from once a week, once every 2 weeks, once every 3 weeks, and once every 4 weeks for a duration of time, wherein the dosing intervals for the first and subsequent dosing cycles are different, thereby treating the advanced or metastatic solid tumor malignancy or lymphoma in the patient.

2. The method of claim 1, wherein the first dosing cycle comprises administration of the mRNA therapeutic at a dosing interval of once a week or once every 2 weeks for a duration of time.

3. The method of claim 1, wherein the first dosing cycle comprises administration of the mRNA therapeutic at a dosing interval of once a week for a duration of time, and at least one subsequent dosing cycle comprising administration of the mRNA therapeutic at a dosing interval of once every 2 weeks, once every 3 weeks or once every 4 weeks for a duration of time.

4. The method of any one of claims 1-3, wherein the at least one subsequent dosing cycle comprises administration of the mRNA therapeutic at a dosing interval of once every 4 weeks for a duration of time.

5. The method of claim 1, wherein the first dosing cycle comprises administration of the mRNA therapeutic at a dosing interval of once every 2 weeks for a duration of time, and the at least one subsequent dosing cycle comprises administration of the mRNA therapeutic at a dosing interval of once every 4 weeks for a duration of time, optionally wherein the subsequent dosing cycle occurs two, three, four, five or six times.

6. The method of any one of claims 1-5, wherein the duration of time for the first dosing cycle and at least one subsequent dosing cycle is 28-42 days.

7. The method of any one of the preceding claims, wherein the advanced or metastatic solid tumor malignancy in the patient is selected from triple negative breast cancer, head and neck squamous cell carcinoma, bladder cancer, non-small cell lung carcinoma and melanoma, and the lymphoma is Non-Hodgkin lymphoma.

8. The method of any one of claims 1-7, wherein the advanced or metastatic solid tumor malignancy or lymphoma is refractory to immune checkpoint inhibitor therapy.

9. The method of any one of claims 1-7, wherein the patient has not received anti-cancer treatment prior to administering the mRNA therapeutic.

10. A method for treating advanced or metastatic solid tumor malignancy in a human patient, comprising administering to the patient by intratumoral injection an effective amount of a lipid nanoparticle (LNP) encapsulated messenger RNA (mRNA) therapeutic agent comprising three mRNA drug substances:
(i) a first mRNA comprising an open reading frame (ORF) encoding a human OX40L polypeptide;
(ii) a second mRNA comprising an ORF encoding a human IL-23 polypeptide; and
(iii) a third mRNA comprising an ORF encoding a human IL-36γ polypeptide,
wherein the patient is administered a dose of 0.10-10.0 mg of the mRNA therapeutic agent, wherein the solid tumor malignancy is selected from the group consisting of: a bladder cancer, an immune checkpoint inhibitor (CPI)-refractory melanoma, a neoadjuvant melanoma, and a non-small cell lung carcinoma, thereby treating the advanced or metastatic solid tumor malignancy in the patient.

11. The method of any one of claims 7-10, wherein (i) the bladder cancer is a urothelial cancer, optionally wherein the patient has received or is receiving platinum-based chemotherapy or the patient is ineligible for platinum-based chemotherapy; or (ii) the bladder cancer is a squamous-cell bladder cancer, optionally wherein the squamous-cell bladder cancer is PD-L1 negative or expresses low levels of PD-L1.

12. The method of claim 10, wherein the bladder cancer or non-small cell lung carcinoma is refractory to immune checkpoint inhibitor therapy.

13. The method of any one of claims 10-12, wherein the dose of the mRNA therapeutic agent is administered to the patient in a dosing regimen selected from 7 to 21 days, 7 to 14 days, 28 days, 21 days, 14 days, and 7 days.

14. The method of any one of claims 10-12, wherein the patient is administered the dose of the mRNA therapeutic agent every 2 weeks, every 3 weeks, or every 4 weeks.

15. The method of any one of claims 10-12, wherein the patient is administered the dose of the mRNA therapeutic (i) in a first dosing cycle comprising a dosing interval of once a week for a duration of time, and at least one subsequent dosing cycle comprising a dosing interval of once a week, once every 2 weeks, once every 3 weeks, or once every 4 weeks, for a duration of time; (ii) in a first dosing cycle comprising a dosing interval of once every 2 weeks for a duration of time, and at least one subsequent dosing cycle comprising a dosing interval of once every 4 weeks for a duration of time; or (iii) in a first dosing cycle comprising a dosing interval of once every 2 weeks for a duration of time, and up to six subsequent dosing cycles each comprising a dosing interval of once every 4 weeks for a duration of time.

16. The method of claim 15, wherein the duration of time for the first dosing cycle and at least one subsequent dosing cycle is 28-42 days.

17. The method of any one of claims 1-16, wherein the patient is administered a dose of the mRNA therapeutic agent selected from: 0.25-8.0 mg; 0.25-4.0 mg; 0.25-2.0 mg; 0.25-1.0 mg, 0.25-5 mg; 0.5-8.0 mg; 0.5-4.0 mg; 0.5-2.0 mg; 0.5-1.0 mg; 1.0-8.0 mg; 1.0-4.0 mg; 1.0-2.0 mg; 2.0-8.0 mg; 2.0-4.0 mg; and 4.0-8.0 mg.

18. The method of any one of claims 1-16, wherein the patient is administered a dose of 0.10 mg, 0.25 mg, 0.50 mg, 1.0 mg, 2.0 mg, 4.0 mg, 8.0 mg or 10.0 mg of the mRNA therapeutic agent.

19. The method of any one of the preceding claims, further comprising administering an effective amount of a PD-1 antagonist, a PD-L1 antagonist or a CTLA-4 antagonist.

20. The method of claim 19, wherein the PD-1 antagonist is an antibody or antigen binding portion thereof that specifically binds to PD-1, wherein the PD-L1 antagonist is an antibody or antigen binding portion thereof that specifically binds to PD-L1, or wherein the CTLA-4 antagonist is an antibody or antigen binding portion thereof that specifically binds to CTLA-4.

21. The method of claim 20, wherein the PD-1 antagonist is selected from the group consisting of nivolumab, pembrolizumab, and pidilizumab, wherein the PD-L1 antagonist is selected from the group consisting of durvalumab, avelumab, and atezolizumab, or wherein the CTLA-4 antagonist is selected from the group consisting of ipilimumab and tremelimumab.

22. The method of any one of claims 19-21, wherein (i) the patient is administered a dose of the PD-1 antagonist, the PD-L1 antagonist or CTLA-4 antagonist every 4 weeks; (ii) the patient is administered a dose of the mRNA therapeutic agent prior to administration of the PD-1 antagonist, PD-L1 antagonist or CTLA-4 antagonist; and/or (iii) wherein the mRNA therapeutic agent and the PD-L1 antagonist or the CTLA-4 antagonist are administered to the patient in a dosing regimen selected from 7 to 28 days, 7 to 21 days, 7 to 14 days, 28 days, 21 days, 14 days, and 7 days, optionally wherein the mRNA therapeutic agent and the PD-L1 antagonist or the CTLA-4 antagonist are administered to the patient in a dosing regimen of 28 days.

23. The method of any one of the preceding claims, wherein an anti-tumor immune response is induced or enhanced in the patient.

24. The method of any one of the preceding claims, wherein the human OX40L polypeptide comprises the amino acid sequence as set forth in SEQ ID NO: 1; the human IL-23 polypeptide comprises the amino acid sequence as set forth in SEQ ID NO: 24; and the human IL-36γ polypeptide comprises the amino acid sequence as set forth in SEQ ID NO: 27.

25. The method of any one of the preceding claims, wherein
(i) the first mRNA encoding a human OX40L polypeptide comprises an ORF comprising a nucleotide sequence at least 80% identical to the nucleotide sequence set forth in SEQ ID NO: 4 or comprises the nucleotide sequence set forth in SEQ ID NO: 4;
(ii) the second mRNA encoding a human IL-23 polypeptide comprises an ORF comprising a nucleotide sequence at least 80% identical to the nucleotide sequence set forth in SEQ ID NO: 25 or comprises the nucleotide sequence set forth in SEQ ID NO: 25; and
(iii) the third mRNA encoding a human IL-36γ polypeptide comprises an ORF comprising a nucleotide sequence at least 80% identical to the nucleotide sequence set forth in SEQ ID NO: 28 or comprises the nucleotide sequence set forth in SEQ ID NO: 28.

26. The method of any one of the preceding claims, wherein each of the first mRNA, second mRNA, and third mRNA comprise a 3′ untranslated region (UTR) comprising at least one microRNA-122 (miR-122) binding site, optionally wherein the miR-122 binding site is a miR-122-3p binding site or a miR-122-5p binding site, further optionally wherein the miR-122-5p binding site comprises the nucleotide sequence set forth in SEQ ID NO: 20, and further optionally wherein the 3′UTR comprises a nucleotide sequence at least 90% identical to the sequence set forth in SEQ ID NO: 17 or comprises the nucleotide sequence as set forth in SEQ ID NO: 17.

27. The method of any one of the preceding claims, wherein each of the first, second, and third mRNAs comprise a 5′cap, a 5′ untranslated region (UTR), and a poly-A tail of about 100 nucleotides in length, optionally wherein the 5′UTR comprises a nucleotide sequence at least 90% identical to the sequence set forth in SEQ ID NO: 16 or comprises the nucleotide sequence as set forth in SEQ ID NO: 16.

28. The method of any one of claims 1-24, wherein
(i) the first mRNA encoding the human OX40L polypeptide comprises a nucleotide sequence at least 90% identical to the nucleotide sequence set forth in SEQ ID NO: 5 or comprises the nucleotide sequence set forth in SEQ ID NO: 5;
(ii) the second mRNA encoding a human IL-23 polypeptide comprises a nucleotide sequence at least 90% identical to the nucleotide sequence set forth in SEQ ID NO: 26 or comprises the nucleotide sequence set forth in SEQ ID NO: 26; and
(iii) the third mRNA encoding a human IL-36γ polypeptide comprises a nucleotide sequence at least 90% identical to the nucleotide sequence set forth in SEQ ID NO: 29 or comprises the nucleotide sequence set forth in SEQ ID NO: 29.

29. The method of any one of the preceding claims, wherein the first, second, and third mRNAs are formulated in the lipid nanoparticle at a mass ratio of OX40L:IL-23:IL-36γ of 1:1:2.

30. The method of any one of the preceding claims, wherein each of the first, second and third mRNAs is chemically modified, optionally wherein each of the first, second, and third mRNAs is fully modified with chemically-modified uridines, optionally wherein the chemically-modified uridines are N1-methylpseudouridines (m1ψ), or each of the first, second and third mRNAs is fully modified with 5-methylcytosine or is fully modified with N1-methylpseudouridines (m1ψ) and 5-methylcytosine.

31. The method of any one of the preceding claims, wherein the LNP comprises an ionizable amino lipid, optionally wherein the ionizable amino lipid comprises a compound having the formula:

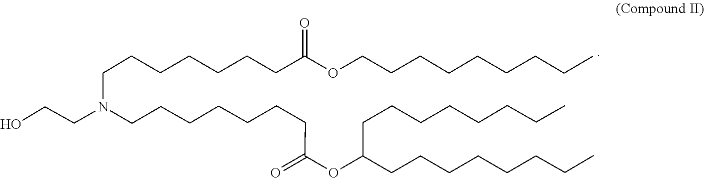 [SMILES: CCCCCCCCCOC(=O)CCCCCCCN(CCO)CCCCCCCC(=O)OC(CCCCCCCC)CCCCCCCC]

32. The method of claim 31, wherein the LNP further comprises a phospholipid, a structural lipid, and a PEG lipid, optionally wherein the LNP comprises a molar ratio of about 20-60% ionizable amino lipid, about 5-25% phospholipid, about 25-55% structural lipid, and about 0.5-1.5% PEG lipid, further optionally wherein the LNP comprises a molar ratio of: (1) 40-60% ionizable amino lipid, 8-16% phospholipid, 30-45% sterol, and 1-5% PEG modified lipid; or (2) 45-65% ionizable amino lipid, 5-10% phospholipid, 25-40% sterol, and 0.5-5% PEG modified lipid.

33. The method of claim 32, wherein the LNP comprises a molar ratio of: (1) 40-60% Compound II, 8-16% DSPC, 30-45% cholesterol, and 1-5% PEG DMG; or (2) 45-65% Compound II, 5-10% DSPC, 25-40% cholesterol, and 0.5-5% PEG DMG.

34. The method of any one of the preceding claims, wherein the mRNA therapeutic agent is administered by a single injection or multiple injections into one or more different sites within the same tumor lesion or divided across several tumor lesions.

35. The method of any one of the preceding claims, wherein the LNP is formulated in a pharmaceutically acceptable carrier, optionally wherein the pharmaceutically acceptable carrier is a solution suitable for intratumoral injection, further optionally wherein the solution comprises a buffer.

36. The method of any one of the preceding claims, wherein the treatment results in:
(i) an anti-tumor immune response in the patient comprising T cell activation, T cell proliferation, and/or T cell expansion, optionally wherein the T cells are CD4+ T cells, CD8+ T cells, or both CD4+ T cells and CD8+ T cells;
(ii) a reduction in size or inhibition of growth of the injected tumor and/or an uninjected tumor;
(iii) an increase in expression of IL-23 and/or IL-36γ in the plasma and/or tumor of the patient;
(iv) an increase in expression of IL-22, IL-6, TNFα, IFNγ and any combination thereof in the plasma and/or tumor of the patient;
(v) an increase in PD-L1 expression in tumor cells and/or immune cells within the tumor microenvironment; or
(vi) any combination of (i)-(v).

37. A kit comprising a container comprising a pharmaceutical composition comprising: an LNP encapsulated mRNA therapeutic agent; and a pharmaceutically acceptable carrier, and instructions for use in treating advanced or metastatic solid tumor malignancy or lymphoma in a human patient, wherein the treatment comprises administration of the pharmaceutical composition by intratumoral injection at a dose of 0.10-10.0 mg of the mRNA therapeutic agent in a dosing regimen comprising a first dosing cycle and at least one subsequent dosing cycle, wherein the first and subsequent dosing cycles comprise administration of the mRNA therapeutic at a dosing interval selected from once a week, once every 2 weeks, once every 3 weeks, and once every 4 weeks for a duration of time, wherein the dosing intervals for the first and subsequent dosing cycles are different, and wherein the LNP encapsulated mRNA therapeutic agent comprises three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide.

38. A kit comprising a container comprising a pharmaceutical composition comprising: an LNP encapsulated mRNA therapeutic agent; and a pharmaceutically acceptable carrier, and instructions for use in treating advanced or metastatic solid tumor malignancy in a human patient, wherein the treatment comprises administration of the pharmaceutical composition by intratumoral injection at a dose of 0.10-10.0 mg of the mRNA therapeutic agent, wherein the solid tumor malignancy is selected from the group consisting of: a bladder cancer, an immune checkpoint inhibitor (CPI)-refractory melanoma, a neoadjuvant melanoma, and a non-small cell lung carcinoma, and wherein the LNP encapsulated mRNA therapeutic agent comprises three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide.

39. The kit of claim 37 or 38, wherein treatment comprises administration of the medicament or pharmaceutical composition in combination with a composition comprising a PD-1 antagonist, a PD-L1 antagonist, or a CTLA-4 antagonist, and an optional pharmaceutically acceptable carrier.

40. The kit of claim 37 or 39, wherein the advanced or metastatic solid tumor malignancy in the patient is selected from triple negative breast cancer, head and neck squamous cell carcinoma, non-small cell lung carcinoma and melanoma, and the lymphoma is Non-Hodgkin lymphoma.

41. An LNP encapsulated mRNA therapeutic agent for use in the manufacture of a medicament for treating advanced or metastatic solid tumor malignancy or lymphoma in a human patient by inducing or enhancing an anti-tumor immune response, wherein the medicament comprises the LNP encapsulated mRNA therapeutic agent, and an optional pharmaceutically acceptable carrier, and wherein the treatment comprises administration of the medicament by intratumoral injection at a dose of 0.10-10.0 mg of the mRNA therapeutic agent in a dosing regimen comprising a first dosing cycle and at least one subsequent dosing cycle, wherein the first and subsequent dosing cycles comprise administration of the mRNA therapeutic at a dosing interval selected from once a week, once every 2 weeks, once every 3 weeks, and once every 4 weeks for a duration of time, wherein the dosing intervals for the first and subsequent dosing cycles are different, and wherein the LNP encapsulated mRNA therapeutic agent comprises three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide.

42. An LNP encapsulated mRNA therapeutic agent for use in the manufacture of a medicament for treating advanced or metastatic solid tumor malignancy or lymphoma in a human patient by inducing or enhancing an anti-tumor immune response, wherein the medicament comprises the LNP encapsulated mRNA therapeutic agent, and an optional pharmaceutically acceptable carrier, and wherein the treatment comprises administration of the medicament by intratumoral injection at a dose of 0.10-10.0 mg of the mRNA therapeutic agent, wherein the solid tumor malignancy is selected from the group consisting of: a bladder cancer, an immune checkpoint inhibitor (CPI)-refractory melanoma, a neoadjuvant melanoma, and a non-small cell lung carcinoma, and wherein the LNP encapsulated mRNA therapeutic agent comprises three mRNA drug substances:
(i) a first mRNA comprising an ORF encoding a human OX40L polypeptide;
(ii) a second mRNA comprising an ORF encoding a human IL-23 polypeptide; and
(iii) a third mRNA comprising an ORF encoding a human IL-36γ polypeptide.

43. The use of claim 41 or 42, wherein treatment comprises administration of the medicament or pharmaceutical composition in combination with a composition comprising a PD-1 antagonist, a PD-L1 antagonist, or a CTLA-4 antagonist, and an optional pharmaceutically acceptable carrier.

44. The use of claim 41 or 43, wherein the advanced or metastatic solid tumor malignancy in the patient is selected from triple negative breast cancer, head and neck squamous cell carcinoma, non-small cell lung carcinoma and melanoma, and the lymphoma is Non-Hodgkin lymphoma.


SEQUENCE LISTING (82 sequences)

SEQ ID NO: 1 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 2 - protein, 133 aa, Homo sapiens
MVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 3 - protein, 198 aa, Mus musculus
MEGEGVQPLDENLENGSRPRFKWKKTLRLVVSGIKGAGMLLCFIYVCLQLSSSPAKDPPIQRLRGAVTRCEDGQLFISSYKNEYQTMEVQNNSVVIKCDGLYIIYLKGSFFQEVKIDLHFREDHNPISIPMLNDGRRIVFTVVASLAFKDKVYLTVNAPDTLCEHLQINDGELIVVQLTPGYCAPEGSYHSTVNQVPL

SEQ ID NO: 4 - RNA, 549 nt, Homo sapiens
auggaaaggguccaaccccuggaagagaaugugggaaaugcagccaggccaagauucgagaggaacaagcuauugcugguggccucuguaauucagggacuggggcugcuccugugcuucaccuacaucugccugcacuucucugcucuucagguaucacaucgguauccucgaauucaaaguaucaaaguacaauuuaccgaauauaagaaggagaaagguuucauccucacuucccaaaaggaggaugaaaucaugaaggugcagaacaacucagucaucaucaacugugauggguuuuaucucaucucccugaagggcuacuucucccaggaagucaacauuagccuucauuaccagaaggaugaggagccccucuuccaacugaagaaggucaggucugucaacuccuugaugguggccucucugacuuacaaagacaaagucuacuugaaugugaccacugacaauaccucccuggaugacuuccaugugaauggcggagaacugauucuuauccaucaaaauccuggugaauucuguguccuu

SEQ ID NO: 5 - RNA, 843 nt, Homo sapiens
  misc_feature (1)..(1): guanine nucleotide methylated at the 7 position
  misc_feature (1)..(2): joined by a triphosphate linkage at their 5′ positions
  misc_feature (2)..(2): 2′ O-methylated
ggggaaauaagagagaaaagaagaguaagaagaaauauaagagccaccauggaaaggguccaaccccuggaagagaaugugggaaaugcagccaggccaagauucgagaggaacaagcuauugcugguggccucuguaauucagggacuggggcugcuccugugcuucaccuacaucugccugcacuucucugcucuucagguaucacaucgguauccucgaauucaaaguaucaaaguacaauuuaccgaauauaagaaggagaaagguuucauccucacuucccaaaaggaggaugaaaucaugaaggugcagaacaacucagucaucaucaacugugauggguuuuaucucaucucccugaagggcuacuucucccaggaagucaacauuagccuucauuaccagaaggaugaggagccccucuuccaacugaagaaggucaggucugucaacuccuugaugguggccucucugacuuacaaagacaaagucuacuugaaugugaccacugacaauaccucccuggaugacuuccaugugaauggcggagaacugauucuuauccaucaaaauccuggugaauucuguguccuuugauaauaggcuggagccucgguggccaugcuucuugccccuugggccuccccccagccccuccuccccuuccugcacccguaccccccaaacaccauugucacacuccaguggucuuugaauaaagucugagugggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 6 - RNA, 3484 nt, Homo sapiens
ggcccugggaccuuugccuauuuucugauugauaggcuuuguuuugucuuuaccuccuucuuucuggggaaaacuucaguuuuaucgcacguuccccuuuuccauaucuucaucuucccucuacccagauugugaagauggaaaggguccaaccccuggaagagaaugugggaaaugcagccaggccaagauucgagaggaacaagcuauugcugguggccucuguaauucagggacuggggcugcuccugugcuucaccuacaucugccugcacuucucugcucuucagguaucacaucgguauccucgaauucaaaguaucaaaguacaauuuaccgaauauaagaaggagaaagguuucauccucacuucccaaaaggaggaugaaaucaugaaggugcagaacaacucagucaucaucaacugugauggguuuuaucucaucucccugaagggcuacuucucccaggaagucaacauuagccuucauuaccagaaggaugaggagccccucuuccaacugaagaaggucaggucugucaacuccuugaugguggccucucugacuuacaaagacaaagucuacuugaaugugaccacugacaauaccucccuggaugacuuccaugugaauggcggagaacugauucuuauccaucaaaauccuggugaauucuguguccuuugaggggcugauggcaauaucuaaaaccaggcaccagcaugaacaccaagcuggggguggacagggcauggauucuucauugcaagugaaggagccucccagcucagccacgugggaugugacaagaagcagauccuggcccucccgcccccaccccucagggauauuuaaaacuuauuuuauauaccaguuaaucuuauuuauccuuauauuuucuaaauugccuagccgucacaccccaagauugccuugagccuacuaggcaccuuugugagaaagaaaaaauagaugccucuucuucaagaugcauuguuucuauuggucaggcaauugucauaauaaacuuaugucauugaaaacgguaccugacuaccauuugcuggaaauuugacauguguguggcauuaucaaaaugaagaggagcaaggagugaaggagugggguuaugaaucugccaaaggugguaugaaccaaccccuggaagccaaagcggccucuccaagguuaaauugauugcaguuugcauauugccuaaauuuaaacuuucucauuugguggggguucaaaagaagaaucagcuugugaaaaaucaggacuugaagagagccgucuaagaaauaccacgugcuuuuuuucuuuaccauuuugcuuucccagccuccaaacauaguuaauagaaauuucccuucaaagaacugucuggggaugugaugcuuugaaaaaucuaaucagugacuuaagagagauuuucuuguauacagggagagugagauaacuuauugugaaggguuagcuuuacuguacaggauagcagggaacuggacaucucaggguaaaagucaguacggauuuuaauagccuggggaggaaaacacauucuuugccacagacaggcaaagcaacacaugcucauccuccugccuaugcugagauacgcacucagcuccaugucuuguacacacagaaacauugcugguuucaagaaaugaggugauccuauuaucaaauucaaucugaugucaaauagcacuaagaaguuauugugccuuaugaaaaauaaugaucucugucuagaaauaccauagaccauauauagucucacauugauaauugaaacuagaagggucuauaaucagccuaugccagggcuucaauggaauaguauccccuuauguuuaguugaaauguccccuuaacuugauauaauguguuaugcuuauggcgcuguggacaaucugauuuuucaugucaacuuuccagaugauuuguaacuucucugugccaaaccuuuuauaaacauaaauuuuugagauauguauuuuaaaauuguagcacauguuucccugacauuuucaauagaggauacaacaucacagaaucuuucuggaugauucuguguuaucaaggaauuguacugugcuacaauuaucucuagaaucuccagaaagguggagggcuguucgcccuuacacuaaauggucucaguuggauuuuuuuuuccuguuuucuauuuccucuuaaguacaccuucaacuauauucccaucccucuauuuuaaucuguuaugaaggaagguaaauaaaaaugcuaaauagaagaaauuguagguaagguaagaggaaucaaguucugaguggcugccaaggcacucacagaaucauaaucauggcuaaauauuuauggagggccuacuguggaccaggcacugggcuaaauacuuacauuuacaagaaucauucugagacagauauucaaugauaucuggcuucacuacucagaagauuguguguguguuuguguguguguguguguguguauuucacuuuuuguuauugaccauguucugcaaaauugcaguuacucagugagugauauccgaaaaaguaaacguuuaugacuauagguaauauuuaagaaaaugcaugguucauuuuuaaguuuggaauuuuuaucuauauuucucacagaugugcagugcacaugcaggccuaaguauauguuguguguguuguuugucuuugaugucaugguccccucucuuaggugcucacucgcuuugggugcaccuggccugcucuucccauguuggccucugcaaccacacagggauauuucugcuaugcaccagccucacuccaccuuccuuccaucaaaaauaugugugugugucucagucccuguaagucauguccuucacagggagaauuaacccuucgauauacauggcagaguuuugugggaaaagaauugaaugaaaagucaggagaucagaauuuuaaauuugacuuagccacuaacuagccauguaaccuugggaaagucauuucccauuucugggucuugcuuuucuuucuguuaaaugagaggaauguuaaauaucuaacaguuuagaaucuuaugcuuacaguguuaucugugaaugcacauauuaaaugucuauguucuuguugcuaugagucaaggaguguaaccuucuccuuuacuauguugaauguauuuuuuucuggacaagcuuacaucuuccucagccaucuuugugaguccuucaagagcaguuaucaauuguuaguuagauauuuucuauuuagagaaugcuuaagggauuccaaucccgauccaaaucauaauuuguucuuaaguauacugggcagguccccuauuuuaagucauaauuuuguauuuagugcuuuccuggcucucagagaguauuaauauugauauuaauaauauaguuaauaguaauauugcuauuuacauggaaacaaauaaaagaucucagaauucacuaaaaaaaaaaa

SEQ ID NO: 7 - RNA, 737 nt, Artificial Sequence
  Human OX40L with 5′-UTR, 3′-UTR, and miR-122 binding site
gggaaauaagagagaaaagaagaguaagaagaaauauaagagccaccauggaaaggguccaaccccuggaagagaaugugggaaaugcagccaggccaagauucgagaggaacaagcuauugcugguggccucuguaauucagggacuggggcugcuccugugcuucaccuacaucugccugcacuucucugcucuucagguaucacaucgguauccucgaauucaaaguaucaaaguacaauuuaccgaauauaagaaggagaaagguuucauccucacuucccaaaaggaggaugaaaucaugaaggugcagaacaacucagucaucaucaacugugauggguuuuaucucaucucccugaagggcuacuucucccaggaagucaacauuagccuucauuaccagaaggaugaggagccccucuuccaacugaagaaggucaggucugucaacuccuugaugguggccucucugacuuacaaagacaaagucuacuugaaugugaccacugacaauaccucccuggaugacuuccaugugaauggcggagaacugauucuuauccaucaaaauccuggugaauucuguguccuuugauaauaggcuggagccucgguggccaugcuucuugccccuugggccuccccccagccccuccuccccuuccugcacccguaccccccaaacaccauugucacacuccaguggucuuugaauaaagucugagugggcggc

SEQ ID NO: 8 - RNA, 549 nt, Homo sapiens
auggagcgggugcagccccuggaggagaacgugggcaacgccgcucggccacgguucgagcggaacaagcugcugcugguggcuagcgugauccagggccugggccugcugcugugcuucaccuacaucugccugcacuucagcgcccugcaggugagccaccgguauccccggauccagagcaucaaggugcaguucaccgaguacaagaaggagaagggcuucauccugaccagccagaaggaggacgagaucaugaaggugcagaacaacagcgugaucaucaacugcgacggcuucuaccugaucagccugaagggcuacuucagccaggaggugaacaucagccugcacuaccagaaggacgaggagccccuguuccagcugaagaaggugcggagcgugaacagccugaugguggccagccugaccuacaaggacaagguguaccugaacgugaccaccgacaacaccagccuggacgacuuccacgugaacggcggcgagcugauccugauccaccagaaccccggcgaguucugcgugcug

SEQ ID NO: 9 - RNA, 549 nt, Homo sapiens
auggaaaggguccaaccccucgaagagaacgugggaaacgcagccaggccaagauucgagaggaacaagcuauugcucguggccucaguaauucagggacucggguuacuccuuugcuucaccuacaucugcuugcacuucagugcucugcagguaucacaucgguauccucgaauucaaaguaucaaaguacaauuuaccgaauauaagaaggagaaagguuucauccucacuucccagaaggaggaugaaaucaugaaggugcagaacaacucagucaucaucaacugugauggguuuuaucucaucucccugaagggcuacuucucccaggaagucaacauuagccuucauuaccagaaggaugaggagccccucuuccaacugaagaaggucaggucugucaacuccuugaugguagccucucugacuuacaaagacaaagucuacuugaaugugaccacugacaauaccucccuggaugacuuccaugugaauggcggagaacugauucuuauccaucagaauccuggugaauucuguguccuu

SEQ ID NO: 10 - RNA, 549 nt, Homo sapiens; identical to SEQ ID NO: 8

SEQ ID NO: 11 - RNA, 57 nt, Unknown
  5′ UTR
aggaaauaagagagaaaagaagaguaagaagaaauauaagaccccggcgccgccacc

SEQ ID NO: 12 - RNA, 85 nt, Homo sapiens
ccuuagcagagcuguggagugugacaaugguguuugugucuaaacuaucaaacgccauuaucacacuaaauagcuacugcuaggc

SEQ ID NO: 13 - RNA, 22 nt, Homo sapiens
aacgccauuaucacacuaaaua

SEQ ID NO: 14 - RNA, 22 nt, Homo sapiens
uauuuagugugauaauggcguu

SEQ ID NO: 15 - RNA, 92 nt, Unknown
  5′ UTR
ucaagcuuuuggacccucguacagaagcuaauacgacucacuauagggaaauaagagagaaaagaagaguaagaagaaauauaagagccacc

SEQ ID NO: 16 - RNA, 47 nt, Unknown
  5′ UTR
gggaaauaagagagaaaagaagaguaagaagaaauauaagagccacc

SEQ ID NO: 17 - RNA, 141 nt, Unknown
  3′ UTR
ugauaauaggcuggagccucgguggccaugcuucuugccccuugggccuccccccagccccuccuccccuuccugcacccguaccccccaaacaccauugucacacuccaguggucuuugaauaaagucugagugggcggc

SEQ ID NO: 18 - RNA, 164 nt, Artificial Sequence
  3′ UTR with mi-122 and mi-142.3p sites
ugauaauaggcuggagccucgguggccaugcuucuugccccuugggcccaaacaccauugucacacuccauccccccagccccuccuccccuuccuccauaaaguaggaaacacuacaugcacccguacccccguggucuuugaauaaagucugagugggcggc

SEQ ID NO: 19 - RNA, 22 nt, Homo sapiens
uggagugugacaaugguguuug

SEQ ID NO: 20 - RNA, 22 nt, Homo sapiens
caaacaccauugucacacucca

SEQ ID NO: 21 - RNA, 7 nt, Unknown
  Kozak Consensus
gccagcc

SEQ ID NO: 22 - RNA, 141 nt, Artificial Sequence
  3′ UTR with miR-122
ugauaauaggcuggagccucgguggccuagcuucuugccccuugggccuccccccagccccuccuccccuuccugcacccguaccccccaaacaccauugucacacuccaguggucuuugaauaaagucugagugggcggc

SEQ ID NO: 23 - DNA, 47 bp, Unknown
  standard UTR
gggaaataagagagaaaagaagagtaagaagaaatataagagccacc

SEQ ID NO: 24 - protein, 505 aa, Artificial Sequence
  human IL-23 (IL-12p40 subunit and IL-23p19 subunit linked by GS Linker)
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 25 - RNA, 1515 nt, Artificial Sequence
  human IL-23 (IL-12p40 subunit and IL-23p19 subunit linked by GS Linker)
augugucaccagcaguuggucaucucuugguuuucccugguauuucuggcaucuccccucguggccauaugggaacugaagaaagauguuuaugucguagaauuggauugguauccggaugccccuggagaaauggugguccucaccugugacaccccugaagaagaugguaucaccuggaccuuggaccagagcagugaggucuuaggcucuggcaagacccugaccauccaagucaaagaguuuggagaugcuggccaguacaccugucacaaaggaggcgagguucuaagccauucgcuccugcugcuucacaagaaggaagauggaauuugguccacugauauuuuaaaggaccagaaagaacccaagaauaagaccuuucuaagaugcgaggccaagaauuauucuggacguuucaccugcugguggcugacgacaaucaguacugauuugacauucagugucaagagcagcagaggcucuucugacccccaaggggugacgugcggagcugcuacacucucugcagagagagucagaggggacaacaaggaguaugaguacucaguggagugccaggaggacagugccugcccagcugcugaggagagucugcccauugaggucaugguggaugccguucacaagcucaaguaugagaacuacaccagcagcuucuucaucagggacaucaucaaaccugacccacccaagaacuugcagcugaagccauuaaagaauucucggcagguggaggucagcugggaguacccugacaccuggaguacuccacauuccuacuucucccugacauucugcguucagguccagggcaagagcaagagagagaagaaagauagagucuucacggacaagaccucagccacggucaucugccgcaagaaugccagcauuagcgugcgggcccaggaccgcuacuauagcucaucuuggagcgaaugggcaucugugcccugcaguggcggagggggcggagggagcagagcugugccugggggcagcagcccugccuggacucagugccagcagcuuucacagaagcucugcacacuggccuggagugcacauccacuagugggacacauggaucuaagagaagagggagaugaagagacuacaaaugauguuccccauauccaguguggagauggcugugacccccaaggacucagggacaacagucaguucugcuugcaaaggauccaccagggucugaucuuuuaugagaagcugcuaggaucggauauuuucacaggggagccuucucugcucccugauagcccugugggccagcuucaugccucccuacugggccucagccaacuccugcagccugagggucaccacugggagacucagcagauuccaagccucagucccagccagccauggcagcgucuccuucuccgcuucaagauccuucgcagccuccaggccuuuguggcuguagccgcccgggucuuugcccauggagcagcaacccugaguccc

SEQ ID NO: 26 - RNA, 1809 nt, Artificial Sequence
  human IL-23 (IL-12p40 subunit and IL-23p19 subunit linked by GS Linker)
  misc_feature (1)..(1): guanine nucleotide methylated at the 7 position
  misc_feature (1)..(2): joined by a triphosphate linkage at their 5′ positions
  misc_feature (2)..(2): 2′ O-methylated
ggggaaauaagagagaaaagaagaguaagaagaaauauaagagccaccaugugucaccagcaguuggucaucucuugguuuucccugguauuucuggcaucuccccucguggccauaugggaacugaagaaagauguuuaugucguagaauuggauugguauccggaugccccuggagaaauggugguccucaccugugacaccccugaagaagaugguaucaccuggaccuuggaccagagcagugaggucuuaggcucuggcaagacccugaccauccaagucaaagaguuuggagaugcuggccaguacaccugucacaaaggaggcgagguucuaagccauucgcuccugcugcuucacaagaaggaagauggaauuugguccacugauauuuuaaaggaccagaaagaacccaagaauaagaccuuucuaagaugcgaggccaagaauuauucuggacguuucaccugcugguggcugacgacaaucaguacugauuugacauucagugucaagagcagcagaggcucuucugacccccaaggggugacgugcggagcugcuacacucucugcagagagagucagaggggacaacaaggaguaugaguacucaguggagugccaggaggacagugccugcccagcugcugaggagagucugcccauugaggucaugguggaugccguucacaagcucaaguaugagaacuacaccagcagcuucuucaucagggacaucaucaaaccugacccacccaagaacuugcagcugaagccauuaaagaauucucggcagguggaggucagcugggaguacccugacaccuggaguacuccacauuccuacuucucccugacauucugcguucagguccagggcaagagcaagagagagaagaaagauagagucuucacggacaagaccucagccacggucaucugccgcaagaaugccagcauuagcgugcgggcccaggaccgcuacuauagcucaucuuggagcgaaugggcaucugugcccugcaguggcggagggggcggagggagcagagcugugccugggggcagcagcccugccuggacucagugccagcagcuuucacagaagcucugcacacuggccuggagugcacauccacuagugggacacauggaucuaagagaagagggagaugaagagacuacaaaugauguuccccauauccaguguggagauggcugugacccccaaggacucagggacaacagucaguucugcuugcaaaggauccaccagggucugaucuuuuaugagaagcugcuaggaucggauauuuucacaggggagccuucucugcucccugauagcccugugggccagcuucaugccucccuacugggccucagccaacuccugcagccugagggucaccacugggagacucagcagauuccaagccucagucccagccagccauggcagcgucuccuucuccgcuucaagauccuucgcagccuccaggccuuuguggcuguagccgcccgggucuuugcccauggagcagcaacccugagucccugauaauaggcuggagccucgguggccaugcuucuugccccuugggccuccccccagccccuccuccccuuccugcacccguaccccccaaacaccauugucacacuccaguggucuuugaauaaagucugagugggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 27 - protein, 172 aa, Artificial Sequence
  hIGKV4-hIL-36gamma construct
MVLQTQVFISLLLWISGAYGSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 28 - RNA, 516 nt, Homo sapiens
augguguugcagacccaggucuucauuucucuguugcucuggaucucuggugccuacgggucaauguguaaaccuauuacugggacuauuaaugauuugaaucagcaaguguggacccuucagggucagaaccuuguggcaguuccacgaagugacagugugaccccagucacuguugcuguuaucacaugcaaguauccagaggcucuugagcaaggcagaggggaucccauuuauuugggaauccagaauccagaaauguguuuguauugugagaagguuggagaacagcccacauugcagcuaaaagagcagaagaucauggaucuguauggccaacccgagcccgugaaacccuuccuuuucuaccgugccaagacugguaggaccuccacccuugagucuguggccuucccggacugguucauugccuccuccaagagagaccagcccaucauucugacuucagaacuugggaagucauacaacacugccuuugaauuaaauauaaaugac

SEQ ID NO: 29 - RNA, 810 nt, Homo sapiens
  misc_feature (1)..(1): guanine nucleotide methylated at the 7 position
  misc_feature (1)..(2): joined by a triphosphate linkage at their 5′ positions
  misc_feature (2)..(2): 2′ O-methylated
ggggaaauaagagagaaaagaagaguaagaagaaauauaagagccaccaugguguugcagacccaggucuucauuucucuguugcucuggaucucuggugccuacgggucaauguguaaaccuauuacugggacuauuaaugauuugaaucagcaaguguggacccuucagggucagaaccuuguggcaguuccacgaagugacagugugaccccagucacuguugcuguuaucacaugcaaguauccagaggcucuugagcaaggcagaggggaucccauuuauuugggaauccagaauccagaaauguguuuguauugugagaagguuggagaacagcccacauugcagcuaaaagagcagaagaucauggaucuguauggccaacccgagcccgugaaacccuuccuuuucuaccgugccaagacugguaggaccuccacccuugagucuguggccuucccggacugguucauugccuccuccaagagagaccagcccaucauucugacuucagaacuugggaagucauacaacacugccuuugaauuaaauauaaaugacugauaauaggcuggagccucgguggccaugcuucuugccccuugggccuccccccagccccuccuccccuuccugcacccguaccccccaaacaccauugucacacuccaguggucuuugaauaaagucugagugggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 30-72 - skipped (43 entries)

SEQ ID NO: 73 - RNA, 22 nt, Homo sapiens
aacgccauuaucacacuaaaua

SEQ ID NO: 74-81 - skipped (8 entries)

SEQ ID NO: 82 - RNA, 22 nt, Homo sapiens
uggagugugacaaugguguuug